(19) United States
(12) Patent Application Publication
(10) Pub. No.: US 2024/0287089 A1
(43) Pub. Date: Aug. 29, 2024

(54) Preparation and Uses of 7-Azaindenoisoquinolines

(71) Applicants: Purdue Research Foundation, West Lafayette, IN (US); Gibson Oncology, LLC, Miami, FL (US)

(72) Inventors: Mark S. Cushman, West Lafayette, IN (US); Danzhou Yang, Lafayette, IN (US); Venkat Chintareddy, Vestavia Hills, AL (US); Pooran Chand, Birmingham, AL (US)

(21) Appl. No.: 18/618,496

(22) Filed: Mar. 27, 2024

Related U.S. Application Data
(63) Continuation-in-part of application No. 18/694,168, filed on Jan. 1, 0001.
(60) Provisional application No. 63/337,248, filed on May 2, 2022.
(60) Provisional application No. 63/351,550, filed on Jun. 13, 2022.

(51) Int. Cl. C07D 491/147; A61K 31/4375

(52) U.S. Cl. CPC C07D 491/147; A61K 31/4375

(57) ABSTRACT

Described herein are new 8,9-dialoxy-7-azaindenoisoquinoline compounds (I), processes for their preparation, and methods of their use in the treatment of diseases responsive to inhibition of topoisomerate I and/or binding to the c-MYC G-Quadruplex.

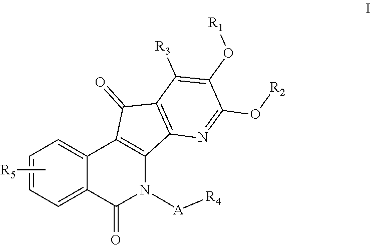

### CROSS REFERENCE TO RELATED APPLICATIONS

This application claims the benefit under 35 USC § 119(e) of U.S. Provisional Application Nos. 63/337,248, filed May 2, 2022, and 63/351,550, filed Jun. 13, 2022, which are incorporated herein by reference.

### GOVERNMENT RIGHTS

This invention was made with government support under U01CA089566, P30CACA023168, U01CA240346, and R01CA177585 awarded by the National Institutes of Health. The government has certain rights in the invention.

### TECHNICAL FIELD

This disclosure relates to novel 7-azaindenoisoquinoline compounds, processes for their preparation, and their use for a variety of therapeutic uses, in particular to treat cancer. This disclosure relates to novel inhibitors of Topoisomerase 1 (Top 1) and compounds that block the activities of MYC oncogene, such as by binding the MYC promoter G-quadruplex. In some embodiments, the disclosure relates to a method for treating a patient with cancer by targeting the MYC oncogene or otherwise blocking the activity of the MYC oncogene. In some embodiments, the disclosure relates to a method for treating a patient with cancer by targeting human topoisomerase I. In some other embodiments, the disclosure relates to a method for treating a patient with cancer by a dual mechanism of action targeting both the MYC oncogene and human topoisomerase I.

### BACKGROUND

Cancer is a group of diverse diseases involving abnormal cell growth. Currently there are more than 100 types of identified cancer that affect human beings as well as animals. In 2018, there were an estimated 1,708,921 new human cancer cases diagnosed and 599,265 cancer deaths in the U.S. alone (Cancer Statistics 2018—Centers for Disease Control and Prevention). There are unmet and increasing needs for novel therapies for cancer treatments.

DNA is the target of many important anticancer agents, including human topoisomerase I inhibitors. Recently there has been significant progress in developing molecular-targeted therapies. A therapeutic advantage can be gained from DNA-targeted drugs with cancer-specific molecular targeting properties. Indenoisoquinolines are human topoisomerase I inhibitors with improved physicochemical and biological properties as compared to the traditional camptothecin topoisomerase I inhibitors that are clinically used for the treatment of various solid tumors. Three indenoisoquinolines, indotecan (LMP400), indimitecan (LMP776), and LMP744, have entered phase I clinical trials in adults with relapsed solid tumors and lymphomas. However, it has been found that some indenoisoquinolines with potent anticancer activity surprisingly did not show strong topoisomerase I inhibition. It has been discovered that certain indenoisoquinolines also target MYC G-quadruplexes (MYC-G4).

MYC is one of the most important oncogenes and is overexpressed in more than 80% of all types of cancer. The transcription factor MYC protein is involved in cell proliferation, differentiation, and apoptosis, and plays a pivotal role in tumor initiation and progression as well as drug resistance. MYC is found to be a general transcriptional “amplifier” in cancer cells. It has been observed that even a brief inhibition of MYC expression may permanently stop tumor growth and induce tumor regression in vivo because of the “oncogene addiction” of tumor cells. Therefore, MYC is a potential therapeutic target. However, the MYC protein is not an easy drug target due to its short half-life and lack of an apparent small molecule binding pocket.

G4s detected in immortalized precancerous cells are at 10 times higher levels than in normal human cells, and G4-sites are found to be specifically enriched in regulatory, transcriptionally active regions of chromatin, particularly the MYC promoter region.

The nuclease hypersensitive element (NHE) 1111 in the MYC promoter, which controls 85-90% of MYC transcriptional activity, forms a DNA G-quadruplex (G4) under transcription-associated negative supercoiling and functions as a transcriptional silencer. It has been discovered that compounds of formula (I) are useful for the treatment of cancers and particularly MYC-positive cancers. It is believed that compounds of formula (I) may target MYC quadruplexes (MYC G4s) and/or topoisomerase I and are effective against many cancers including MYC-positive cancers that contain MYC G4.

### SUMMARY

In accordance with some embodiments, the present disclosure provides a compound of formula (I), or a salt, hydrate, or solvate thereof,

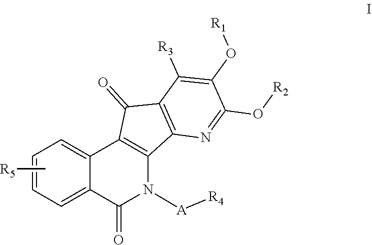

wherein
R₁ and R₂ are independently selected from the group consisting of hydrogen, (C₁-C₆) alkyl, (C₂-C₆)alkenyl, (C₂-C₆)alkynyl, optionally substituted heteroalkyl, and optionally substituted acyl, or R₁ and R₂ together with the atoms to which they are attached form a 5-membered or 6-membered ring;
R₃ is hydrogen, halo, nitro, cyan, CF₃, (C₁-C₆)alkyl, (C₁-C₆)alkylthio, or (C₁-C₆)alkoxy;
A is alkylene;
R₄ is selected from the group consisting of heteroaryl, heterocyclyl, heterocyclylamino, amino, hydroxyl, halo, cyano, alkylamino, dialkylamino, hydroxyalkylamino, bis(hydroxyalkyl)amino, and hydroxyalkylaminoalkylamino, wherein each of heteroaryl, heterocyclyl, and heterocyclylamino is optionally substituted;
R₅ represents from 1 to 2 substituents independently selected from the group consisting of amino, (C₁-C₆)alkylamino, di(C₁-C₆)alkylamino, hydroxy (C₁-C₆)alkyl, (C₁-C₆)alkenyl, (C₁-C₆)alkynyl, (C₁-C₆)heteroalkyl, (C₃-C₈)cycloalkyl, (C₃-C₈)cycloheteroalkyl, (C₁-C₆)alkoxy, (C₁-C₆)alkyl(CO)O—, (C₁-C₆)alkyl-O(CO)O— and (C₁-C₆)alkylthio; or R₅ represents 2 adjacent substituents that are taken together with the attached carbons to form an optionally substituted cycle or heterocycle.

In some embodiments, A is (CH₂)n, wherein n is selected from the group consisting of 1, 2 and 3. In some embodiments, A is (CH₂)₃.

In accordance with some embodiments, R₃ is hydrogen.

In accordance with some embodiments, R₁ and R₂ are independently selected from the group consisting of hydrogen and CH₃. In other embodiments, R₁ and R₂ taken together with atoms they are attached to form a cycle.

In some embodiments, R₄ is selected from the group consisting of heteroaryl, heterocyclyl, heterocyclylamino, amino, hydroxyl, halo, cyano, alkylamino, dialkylamino, hydroxyalkylamino, bis(hydroxyalkyl)amino, and hydroxyalkylaminoalkylamino, wherein each of heteroaryl, heterocyclyl, and heterocyclylamino is optionally substituted.

In some embodiments, R₁ and R₂ are taken together to form —CH₂—, R₃ is hydrogen, R₅ represents 2-MeO and 3-MeO, A is (CH₂)₃, and R₄ is selected from the group consisting of heteroaryl, heteroaryloxy, heteroarylamino, heteroarylalkylaminoalkylamino, heterocyclyl, heterocyclylamino, amino, hydroxyl, halo, cyano, alkylamino, dialkylamino, trialkylammonium, hydroxyalkylamino, bis(hydroxyalkyl)amino, and hydroxyalkylaminoalkylamino, wherein each of heteroaryl, heteroaryloxy, and heteroarylamino, heteroarylalkylaminoalkylamino, heterocyclyl, and heterocyclylamino is optionally substituted.

In accordance with some embodiments, the compound of formula (I) is selected from the group consisting of

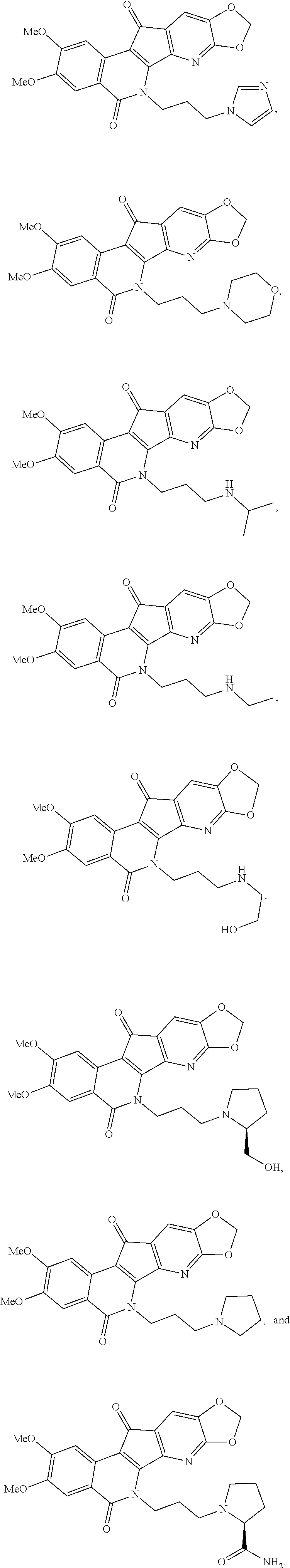

In some embodiments, the present disclosure provides a pharmaceutical composition comprising a compound of formula (I), or the salt, hydrate, or solvate thereof, wherein formula (I) is as herein described.

In accordance with some embodiments, the present disclosure provides a pharmaceutical composition comprising a compound of formula (I), or a salt, hydrate, or solvate thereof

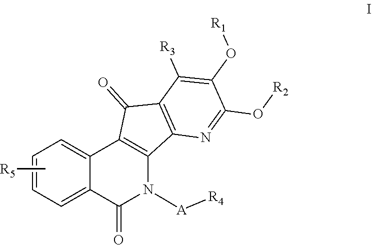

wherein
R₁ and R₂ are independently selected from the group consisting of hydrogen, (C₁-C₆) alkyl, (C₂-C₆)alkenyl, (C₂-C₆)alkynyl, optionally substituted heteroalkyl, and optionally substituted acyl, or R₁ and R₂ together with the atoms to which they are attached form a 5-membered or 6-membered ring;
R₃ is hydrogen, halo, nitro, cyan, CF₃, (C₁-C₆)alkyl, (C₁-C₆)alkylthio, or (C₁-C₆)alkoxy;
A is alkylene;
R₄ is selected from the group consisting of heteroaryl, heterocyclyl, heterocyclylamino, amino, hydroxyl, halo, cyano, alkylamino, dialkylamino, hydroxyalkylamino, bis(hydroxyalkyl)amino, and hydroxyalkylaminoalkylamino, wherein each of heteroaryl, heterocyclyl, and heterocyclylamino is optionally substituted;
R₅ represents from 1 to 2 substituents independently selected from the group consisting of amino, (C₁-C₆)alkylamino, di(C₁-C₆)alkylamino, hydroxy (C₁-C₆)alkyl, (C₁-C₆)alkenyl, (C₁-C₆)alkynyl, (C₁-C₆)heteroalkyl, (C₃-C₈)cycloalkyl, (C₃-C₈)cycloheteroalkyl, (C₁-C₆)alkoxy, (C₁-C₆)alkyl(CO)O—, (C₁-C₆)alkyl-O(CO)O— and (C₁-C₆)alkylthio; or R₅ represents 2 adjacent substituents that are taken together with the attached carbons to form an optionally substituted cycle or heterocycle.

In some embodiments, the pharmaceutical composition further comprises at least one additional component selected from the group consisting of a diluent, an excipient, and combinations thereof.

In accordance with embodiments, the pharmaceutical composition is for treating cancer.

In accordance with some embodiments, the pharmaceutical composition comprises a therapeutically effective amount of the compound of formula (I), or salt, hydrate, or solvate thereof.

In accordance with embodiments of the present disclosure, a method for treating a disease responsive to topoisomerase I inhibition or binding to a MYC quadruplex in a host animal is provided. In some embodiments, the method comprises the step of administering to the host animal a composition comprising a therapeutically effective amount of one or more compounds of formula (I), or a salt, hydrate, or solvate thereof, or a pharmaceutical composition comprising one or more compounds of formula (I), or a salt, hydrate, or solvate thereof,
wherein formula (I) is

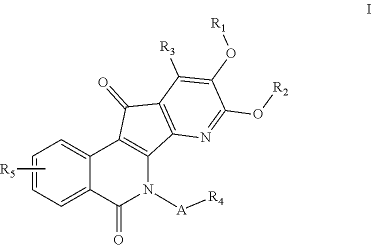

wherein
R₁ and R₂ are independently selected from the group consisting of hydrogen, (C₁-C₆) alkyl, (C₂-C₆)alkenyl, (C₂-C₆)alkynyl, optionally substituted heteroalkyl, and optionally substituted acyl, or R₁ and R₂ together with the atoms to which they are attached form a 5-membered or 6-membered ring;
R₃ is hydrogen, halo, nitro, cyan, CF₃, (C₁-C₆)alkyl, (C₁-C₆)alkylthio, or (C₁-C₆)alkoxy;
A is alkylene;
R₄ is selected from the group consisting of heteroaryl, heterocyclyl, heterocyclylamino, amino, hydroxyl, halo, cyano, alkylamino, dialkylamino, hydroxyalkylamino, bis(hydroxyalkyl)amino, and hydroxyalkylaminoalkylamino, wherein each of heteroaryl, heterocyclyl, and heterocyclylamino is optionally substituted;
R₅ represents from 1 to 2 substituents independently selected from the group consisting of amino, (C₁-C₆)alkylamino, di(C₁-C₆)alkylamino, hydroxy (C₁-C₆)alkyl, (C₁-C₆)alkenyl, (C₁-C₆)alkynyl, (C₁-C₆)heteroalkyl, (C₃-C₈)cycloalkyl, (C₃-C₈)cycloheteroalkyl, (C₁-C₆)alkoxy, (C₁-C₆)alkyl(CO)O—, (C₁-C₆)alkyl-O(CO)O— and (C₁-C₆)alkylthio; or R₅ represents 2 adjacent substituents that are taken together with the attached carbons to form an optionally substituted cycle or heterocycle, and
wherein the pharmaceutical composition optionally further comprises one or more carriers, diluents, or excipients, or a combination thereof.

In some embodiments, the host animal is a human.

In accordance with embodiments of the present disclosure, a process for preparing a compound of formula (I), as herein described, is provided. In some embodiments, the process comprises the step of brominating a compound of formula II to yield compound III where R₁, R₂, and R₃ are as defined in claim 1.

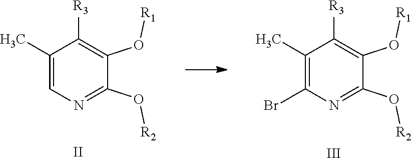

In some embodiment, R₃ is hydrogen.

In some embodiments, where R₁ and R₂ are taken together to form —CH₂—.

In some embodiments, the brominating step comprises treating a solution of the compound of formula (II) in acetic acid with N-bromosuccinimide.

### BRIEF DESCRIPTION OF THE DRAWINGS

FIG. 1 is a schematic of MycG4;

FIG. 2 is a graph showing the increase in melting temperature (ΔTₘ) or MycG4 G-quadruplex in the presence of 7-aza-8,9-methylenedioxyindenoisoquinolines using a FRET-based melting experiment, at conditions: 0.2 μM 5′-g-FAM/3′-TAMRA-MycG4 DNA, 2.0 μM (10 equiv) ligand, in 10 mM K⁺ buffer, pH 7.0;

FIG. 3 is a graph showing reference compound results for the data in FIG. 2;

FIG. 4 shows the imino proton region of 1D ¹H NMR titrations of 7-aza-8,9-methylenedioxyindenoisoquinolines to MycG4 DNA, at conditions: 25° C., 150 μM DNA, 100 mM K⁺ buffer, pH 7.0;

FIG. 5 is a Western Blot showing Myc protein levels with and without 7-aza-8,9-methylenedioxyindenoisoquinoline treatments at 0.5, 1.0, and 2.0 μM for 24 h in MCF-7 cells, with GAPDH used as the loading control;

FIG. 6 illustrates qRT-PCR results showing Myc mRNA levels with and without drug treatments at 1.0 μM for 6 h in MCF-7 cells, wherein relative Myc mRNA levels were calculated by normalizing against the endogenous control GAPDH, all datapoints of five biological repeats were plotted, with each containing duplicate repeats, and the statistical significance between each treatment group and the control is evaluated by one-way ANOVA with Dunnett (**** represents P<0.0001);

FIG. 7 illustrates the reference compound results for the data in FIG. 6;

FIG. 8 illustrates MTS single-dosage assay results showing % viability values (calculated by normalizing blank-subtracted values against the control of the same cell line) of CA-46 and Raji cells with and without 250 nM 7-aza-8,9-methylenedioxyindenoisoquinoline treatments, wherein each bar represents an average of three biological repeats each containing four technical replicates and treatment groups with statistically significant different % viability values between CA-46 and Raji cells are labelled, with p-value<0.005 evaluated by Welch t-tests;

FIG. 9 illustrates the reference compound results for the data in FIG. 8 with and without 250 nM indenoisoquinoline treatments;

FIG. 10 is a plot of tumor volume as a function of time in NOD SCID mice;

FIG. 11 illustrates the effects of 7-aza-8,9-methylenedioxyindenoisoquinoline compounds for Topol inhibition by (A) Topol-mediated relaxation assay using native agarose gel stained with ethidium bromide measuring Topol relaxation activity, and (B) Topol-Mediated DNA Cleavage Assay using agarose gel containing ethidium bromide for optimal visualization of Topol/DNA cleavage complex formation by respective drug compounds, wherein Lane 1 is a DNA relaxation mark, Lane 2 is supercoiled DNA, Lane 3 is supercoiled DNA+Topol (CON), and Lanes 4-11 are supercoiled DNA+Topol+respective compounds at 10 μM;

FIG. 12 is a plot of growth percentages as a function of time after administration of G103, G104, and G105 to female NOD SCID mice in the low and high dose ranges;

FIG. 13 is a plot of growth percentages as a function of time after administration of G103, G104, and G105 to female NOD SCID mice in the medium dose range;

FIG. 14 is a plot of body weights as a function of time after administration of G103, G104, G105, and JQ-1 to female NOD SCID mice after inoculation with MOLT-4 tumor cells;

FIG. 15 is a plot of tumor size versus time after administration of G103, G104, G105, and JQ-1 to female NOD SCID mice after inoculation with MOLT-4 tumor cells;

FIG. 16 is a plot of body weights as a function of time after administration of G103, G104, G105 and JQ-1 to female NOD SCID mice after inoculation with RD-ES tumor cells;

FIG. 17 is a plot of tumor size as a function of time after administration of G103, G104, G105 and JQ-1 to female NOD SCID mice after inoculation with RD-ES tumor cells;

FIG. 18 is a plot of body weights as a function of time after administration of G103 and G105 to female NOD SCID mice after inoculation with RD-ES tumor cells;

FIG. 19 is a plot of tumor size as a function of time after administration of G103 and G105 to female NOD SCID mice after inoculation with RD-ES tumor cells;

FIG. 20 is a plot of body weights as a function of time after administration of G103, G104, G105 and JQ-1 to female NOD SCID mice after inoculation with RS4;11 tumor cells; and

FIG. 21 is a plot of tumor size as a function of time after administration of G103, G104, G105 and JQ-1 to female NOD SCID mice after inoculation with RS4;11 tumor cells.

### DETAILED DESCRIPTION

### Definitions

In each of the embodiments, unless otherwise indicated, it is to be understood that the transitional phrase “consisting essentially of” means that the scope of the corresponding composition, unit dose, method or use is understood to encompass the specified compounds or recited steps, and those that do not materially affect the basic and novel characteristics of the invention described herein. For example, a method described herein that consists essentially of a single compound, or genus of compounds, is understood to represent a monotherapy for the recited disease. Though the monotherapy may include co-administration of one or more carriers, vehicles, diluents, adjuvants, excipients, and the like, and combinations thereof, and/or include co-administration of one or more additional active pharmaceutical ingredients, those latter additional active pharmaceutical ingredients are to be understood to be for treating diseases and/or symptoms distinct from treating the underlying conditions described herein, such as the cancer itself.

In each of the embodiments, unless otherwise indicated, it is to be understood that the formulae include and represent not only all pharmaceutically acceptable salts of the compounds, but also any and all hydrates and/or solvates of the compound formulae. It is appreciated that certain functional groups, such as the hydroxy, amino, and like groups form complexes and/or coordination compounds with water and/or various solvents, in the various physical forms of the compounds. Accordingly, the formulae are to be understood to be a description of such hydrates and/or solvates, including pharmaceutically acceptable solvates.

As used herein, the term “solvates” refers to compounds complexed with a solvent molecule. Compounds may form such complexes with solvents by simply mixing the compounds with a solvent or dissolving the compounds in a solvent. Where the compounds are to be used as pharmaceuticals, such solvents are pharmaceutically acceptable solvents. Where the compounds are to be used as pharmaceuticals, the relative amount of solvent that forms the solvate should be less than established guidelines for such pharmaceutical uses, such as less than International Conference on Harmonization (ICH) Guidelines. Solvates may be isolated from excess solvent by evaporation, precipitation, and/or crystallization. In some embodiments, the solvates are amorphous, and in other embodiments, the solvates are crystalline.

In each of the embodiments, unless otherwise indicated, it is also to be understood that the formulae include and represent any and all crystalline forms, partially crystalline forms, and non-crystalline and/or amorphous forms of the compounds, including partially ordered forms, disordered forms, liquid crystal forms, and meso phases of any of the foregoing.

In each of the embodiments, unless otherwise indicated, it is also to be understood that the formulae include and represent each possible isomer, such as stereoisomers and geometric isomers, both individually and in any and all possible mixtures.

A pharmaceutical composition comprising a compound of any of the embodiments recited herein, and optionally comprising one or more carriers, diluents, excipients, and the like, and combinations thereof.

Illustrative derivatives include, but are not limited to, both those compounds that may be synthetically prepared from the compounds described, as well as those compounds that may be prepared in a similar way as those described, but differing in the selection of starting materials. For example, described are compounds of formula (I) that include various functional groups on aromatic rings, such as R₃ and R₅. Derivatives of those compounds also include the compounds having, for example, different functional groups on those aromatic rings than those explicitly set forth in the definition of formula (I). In addition, derivatives of those compounds also include the compounds having those same or different functional groups at different positions on the aromatic ring. Similarly, derivatives include parallel variations of other functional groups on the compounds described herein, such as R₄.

Such derivatives may include prodrugs of the compounds and compounds that include one or more protection or protecting groups, including compounds that are used in the preparation of other compounds described.

Illustrative analogs include, but are not limited to, those compounds that share functional and, in some cases, structural similarity to those compounds described herein. For example, described are compounds of formula (I) that include a 7-aza-indenoisoquinoline ring system. Illustrative analogs include, but are not limited to, the corresponding ring expanded compounds. Other illustrative analogs include, but are not limited to, the corresponding ring systems that include additional heteroatoms.

It is to be understood that each of the embodiments may be combined in chemically relevant ways to generate subsets of the embodiments. Accordingly, it is to be further understood that all such subsets are also illustrative embodiments.

The compounds described herein may contain one or more chiral centers or may otherwise be capable of existing as multiple stereoisomers. In one embodiment, the disclosure is not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. Such mixtures of stereoisomers may include a single stereochemical configuration at one or more chiral centers, while including mixtures of stereochemical configuration at one or more other chiral centers.

Similarly, the compounds may include geometric centers, such as cis, trans, E, and Z double bonds, or spatial arrangements, such as cis, trans, syn, and anti, on a ring. In another embodiment, the disclosure is not limited to any particular geometric isomer requirement, and that the compounds, and compositions, methods, uses, and medicaments that include them may be pure, or may be any of a variety of geometric isomer mixtures. Such mixtures of geometric isomers may include a single configuration at one or more double bonds, while including mixtures of geometry at one or more other double bonds.

As used herein, the term “alkyl” includes a chain of carbon atoms, which is optionally branched. As used herein, the terms “alkenyl” and “alkynyl” each include a chain of carbon atoms, which is optionally branched, and include at least one double bond or triple bond, respectively. Alkynyl may also include one or more double bonds. In certain embodiments, alkyl is advantageously of limited length, including C₁-C₂₄, C₁-C₁₂, C₁-C₈, C₁-C₆, and C₁-C₄, and C₂-C₂₄, C₂-C₁₂, C₂-C₈, C₂-C₆, and C₂-C₄, and the like Illustratively, such particularly limited length alkyl groups, including C₁-C₈, C₁-C₆, and C₁-C₄, and C₂-C₈, C₂-C₆, and C₂-C₄, and the like may be referred to as lower alkyl. In certain embodiments alkenyl and/or alkynyl may each be advantageously of limited length, including C₂-C₂₄, C₂-C₁₂, C₂-C₈, C₂-C₆, and C₂-C₄, and C₃-C₂₄, C₃-C₁₂, C₃-C₈, C₃-C₆, and C₃-C₄, and the like Illustratively, such particularly limited length alkenyl and/or alkynyl groups, including C₂-C₈, C₂-C₆, and C₂-C₄, and C₃-C₈, C₃-C₆, and C₃-C₄, and the like may be referred to as lower alkenyl and/or alkynyl. Shorter alkyl, alkenyl, and/or alkynyl groups may add less lipophilicity to the compound and accordingly will have different pharmacokinetic behavior. In certain embodiments, the recitation of alkyl refers to alkyl and optionally lower alkyl. In certain embodiments, the recitation of alkenyl refers to alkenyl and optionally lower alkenyl. In certain embodiments, the recitation of alkynyl refers to alkynyl and optionally lower alkynyl. Illustrative alkyl, alkenyl, and alkynyl groups include, but not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like, and the corresponding groups containing one or more double and/or triple bonds, or a combination thereof.

The term “alkylene” includes a divalent chain of carbon atoms, which is optionally branched. The term “alkenylene” and “alkynylene” includes a divalent chain of carbon atoms, which is optionally branched, and includes at least one double bond or triple bond, respectively. Alkynylene may also include one or more double bonds. In certain embodiments, alkylene is advantageously of limited length, including C₁-C₂₄, C₁-C₁₂, C₁-C₈, C₁-C₆, and C₁-C₄, and C₂-C₂₄, C₂-C₁₂, C₂-C₈, C₂-C₆, and C₂-C₄, and the like. Illustratively, such particularly limited length alkylene groups, including C₁-C₈, C₁-C₆, and C₁-C₄, and C₂-C₈, C₂-C₆, and C₂-C₄, and the like may be referred to as lower alkylene. In certain embodiments alkenylene and/or alkynylene may each be advantageously of limited length, including C₂-C₂₄, C₂-C₁₂, C₂-C₈, C₂-C₆, and C₂-C₄, and C₃-C₂₄, C₃-C₁₂, C₃-C₈, C₃-C₆, and C₃-C₄, and the like. Illustratively, such particularly limited length alkenylene and/or alkynylene groups, including C₂-C₈, C₂-C₆, and C₂-C₄, and C₃-C₈, C₃-C₆, and C₃-C₄, and the like may be referred to as lower alkenylene and/or alkynylene. Shorter alkylene, alkenylene, and/or alkynylene groups may add less lipophilicity to the compound and accordingly will have different pharmacokinetic behavior. In certain embodiments, the recitation of alkylene, alkenylene, and alkynylene refers to alkylene, alkenylene, and alkynylene, and optionally lower alkylene, alkenylene, and alkynylene. Illustrative alkyl groups include, but not limited to, methylene, ethylene, n-propylene, isopropylene, n-butylene, isobutylene, sec-butylene, pentylene, 1,2-pentylene, 1,3-pentylene, hexylene, heptylene, octylene, and the like.

As used herein, the term “cycloalkyl” includes a chain of carbon atoms, which is optionally branched, where at least a portion of the chain is cyclic. Cycloalkylalkyl is a subset of cycloalkyl. Cycloalkyl may be polycyclic. Illustrative cycloalkyl groups include, but are not limited to, cyclopropyl, cyclopentyl, cyclohexyl, 2-methylcyclopropyl, cyclopentyleth-2-yl, adamantyl, and the like. As used herein, the term “cycloalkenyl” includes a chain of carbon atoms, which is optionally branched, and includes at least one double bond, where at least a portion of the chain in cyclic. The one or more double bonds may be in the cyclic portion of cycloalkenyl and/or the non-cyclic portion of cycloalkenyl. Cycloalkenylalkyl and cycloalkylalkenyl are each subsets of cycloalkenyl. Cycloalkyl may be polycyclic. Illustrative cycloalkenyl groups include, but are not limited to, cyclopentenyl, cyclohexylethen-2-yl, cycloheptenylpropenyl, and the like. Chain-forming cycloalkyl and/or cycloalkenyl is advantageously of limited length, including C₃-C₂₄, C₃-C₁₂, C₃-C₈, C₃-C₆, and C₅-C₆. Shorter alkyl and/or alkenyl chains forming cycloalkyl and/or cycloalkenyl, respectively, may add less lipophilicity to the compound and accordingly will have different pharmacokinetic behavior.

The term “heteroalkyl” includes a chain of atoms that includes both carbon and at least one heteroatom and is optionally branched. Illustrative heteroatoms include nitrogen, oxygen, and sulfur. In certain variations, illustrative heteroatoms also include phosphorus, and selenium. The term “cycloheteroalkyl” including heterocyclyl and heterocycle, includes a chain of atoms that includes both carbon and at least one heteroatom, such as heteroalkyl, and is optionally branched, where at least a portion of the chain is cyclic. Illustrative heteroatoms include nitrogen, oxygen, and sulfur. In certain variations, illustrative heteroatoms also include phosphorus, and selenium. Illustrative cycloheteroalkyl include, but are not limited to, tetrahydrofuryl, pyrrolidinyl, tetrahydropyranyl, piperidinyl, morpholinyl, piperazinyl, homopiperazinyl, quinuclidinyl, and the like.

The term “aryl” includes monocyclic and polycyclic aromatic carbocyclic groups, each of which may be optionally substituted. Illustrative aromatic carbocyclic groups described herein include, but are not limited to, phenyl, naphthyl, and the like. The term “heteroaryl” includes aromatic heterocyclic groups, each of which may be optionally substituted. Illustrative aromatic heterocyclic groups include, but are not limited to, pyridinyl, pyrimidinyl, pyrazinyl, triazinyl, tetrazinyl, quinolinyl, quinazolinyl, quinoxalinyl, thienyl, pyrazolyl, imidazolyl, oxazolyl, thiazolyl, isoxazolyl, isothiazolyl, oxadiazolyl, thiadiazolyl, triazolyl, benzimidazolyl, benzoxazolyl, benzthiazolyl, benzisoxazolyl, benzisothiazolyl, and the like.

The term “amino” includes the group NH₂, alkylamino, and dialkylamino, where the two alkyl groups in dialkylamino may be the same or different, i.e., alkylalkylamino. Illustratively, amino includes methylamino, ethylamino, dimethylamino, methylethylamino, and the like. In addition, when amino modifies or is modified by another term, such as aminoalkyl, or acylamino, the above variations of the term amino are included. Illustratively, aminoalkyl includes H₂N-alkyl, methylaminoalkyl, ethylaminoalkyl, dimethylaminoalkyl, methylethylaminoalkyl, and the like. Illustratively, acylamino includes acylmethylamino, acylethylamino, and the like.

The term “amino and derivatives thereof” includes amino as described herein, and alkylamino, alkenylamino, alkynylamino, heteroalkylamino, heteroalkenylamino, heteroalkynylamino, cycloalkylamino, cycloalkenylamino, cycloheteroalkylamino, cycloheteroalkenylamino, arylamino, arylalkylamino, arylalkenylamino, arylalkynylamino, heteroarylamino, heteroarylalkylamino, heteroarylalkenylamino, heteroarylalkynylamino, acylamino, and the like, each of which is optionally substituted. The term “amino and derivatives thereof” also includes alkylalkylamino, alkylalkenylamino, alkylalkynylamino, alkylheteroalkylamino, alkylheteroalkenylamino, alkylheteroalkynylamino, alkylcycloalkylamino, alkylcycloalkenylamino, alkylcycloheteroalkylamino, alkylcycloheteroalkenylamino, arylamino, arylalkylamino, arylalkenylamino, arylalkynylamino, heteroarylamino, heteroarylalkylamino, heteroarylalkenylamino, heteroarylalkynylamino, alkylacylamino, and the like, each of which is optionally substituted. The term “amino derivative” also includes urea, carbamate, and the like.

The term “hydroxy and derivatives thereof” includes OH, and alkyloxy, alkenyloxy, alkynyloxy, heteroalkyloxy, heteroalkenyloxy, heteroalkynyloxy, cycloalkyloxy, cycloalkenyloxy, cycloheteroalkyloxy, cycloheteroalkenyloxy, aryloxy, arylalkyloxy, arylalkenyloxy, arylalkynyloxy, heteroaryloxy, heteroarylalkyloxy, heteroarylalkenyloxy, heteroarylalkynyloxy, acyloxy, and the like, each of which is optionally substituted. The term “hydroxy derivative” also includes carbamate, and the like.

The term “thio and derivatives thereof” includes SH, and alkylthio, alkenylthio, alkynylthio, heteroalkylthio, heteroalkenylthio, heteroalkynylthio, cycloalkylthio, cycloalkenylthio, cycloheteroalkylthio, cycloheteroalkenylthio, arylthio, arylalkylthio, arylalkenylthio, arylalkynylthio, heteroarylthio, heteroarylalkylthio, heteroarylalkenylthio, heteroarylalkynylthio, acylthio, and the like, each of which is optionally substituted. The term “thio derivative” also includes thiocarbamate, and the like.

The term “acyl” includes formyl, and alkylcarbonyl, alkenylcarbonyl, alkynylcarbonyl, heteroalkylcarbonyl, heteroalkenylcarbonyl, heteroalkynylcarbonyl, cycloalkylcarbonyl, cycloalkenylcarbonyl, cycloheteroalkylcarbonyl, cycloheteroalkenylcarbonyl, arylcarbonyl, arylalkylcarbonyl, arylalkenylcarbonyl, arylalkynylcarbonyl, heteroarylcarbonyl, heteroarylalkylcarbonyl, heteroarylalkenylcarbonyl, heteroarylalkynylcarbonyl, acylcarbonyl, and the like, each of which is optionally substituted.

The term “carbonyl and derivatives thereof” includes the group C(O), C(S), C(NH) and substituted amino derivatives thereof.

The term “carboxylic acid and derivatives thereof” includes the group CO₂H and salts thereof, and esters and amides thereof, and CN.

The term “sulfinic acid or a derivative thereof” includes SO₂H and salts thereof, and esters and amides thereof.

The term “sulfonic acid or a derivative thereof” includes SO₃H and salts thereof, and esters and amides thereof.

The term “sulfonyl” includes alkylsulfonyl, alkenylsulfonyl, alkynylsulfonyl, heteroalkylsulfonyl, heteroalkenylsulfonyl, heteroalkynylsulfonyl, cycloalkylsulfonyl, cycloalkenylsulfonyl, cycloheteroalkylsulfonyl, cycloheteroalkenylsulfonyl, arylsulfonyl, arylalkylsulfonyl, arylalkenylsulfonyl, arylalkynylsulfonyl, heteroarylsulfonyl, heteroarylalkylsulfonyl, heteroarylalkenylsulfonyl, heteroarylalkynylsulfonyl, acylsulfonyl, and the like, each of which is optionally substituted.

The term “hydroxylamino and derivatives thereof” includes NHOH, and alkyloxylNH, alkenyloxylNH, alkynyloxylNH, heteroalkyloxylNH, heteroalkenyloxylNH, heteroalkynyloxylNH, cycloalkyloxylNH, cycloalkenyloxylNH, cycloheteroalkyloxylNH, cycloheteroalkenyloxylNH, aryloxylNH, arylalkyloxylNH, arylalkenyloxylNH, arylalkynyloxylNH, heteroaryloxylNH, heteroarylalkyloxylNH, heteroarylalkenyloxylNH, heteroarylalkynyloxylNH, acyloxyNH, and the like, each of which is optionally substituted.

The term “hydrazino and derivatives thereof” includes alkylNHNH, alkenylNHNH, alkynylNHNH, heteroalkylNHNH, heteroalkenylNHNH, heteroalkynylNHNH, cycloalkylNHNH, cycloalkenylNHNH, cycloheteroalkylNHNH, cycloheteroalkenylNHNH, arylNHNH, arylalkylNHNH, arylalkenylNHNH, arylalkynylNHNH, heteroarylNHNH, heteroarylalkylNHNH, heteroarylalkenylNHNH, heteroarylalkynylNHNH, acylNHNH, and the like, each of which is optionally substituted.

The term “optionally substituted” includes the replacement of hydrogen atoms with other functional groups on the radical that is optionally substituted. Such other functional groups illustratively include, but are not limited to, amino, hydroxyl, halo, thiol, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxyl, thiol, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

The terms “optionally substituted aryl” and “optionally substituted heteroaryl” include the replacement of hydrogen atoms with other functional groups on the aryl or heteroaryl that is optionally substituted. Such other functional groups, also referred to herein as aryl substituents or heteroaryl substituents, respectively, illustratively include, but are not limited to, amino, hydroxy, halo, thio, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxy, thio, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

Illustrative substituents also include, but are not limited to, a radical —(CH₂)ₓZ_{X}, where x is an integer from 0-6 and Z_{X} is selected from halogen, hydroxy, alkanoyloxy, including C₁-C₆ alkanoyloxy, optionally substituted aroyloxy, alkyl, including C₁-C₆ alkyl, alkoxy, including C₁-C₆ alkoxy, cycloalkyl, including C₃-C₈ cycloalkyl, cycloalkoxy, including C₃-C₈ cycloalkoxy, alkenyl, including C₂-C₆ alkenyl, alkynyl, including C₂-C₆ alkynyl, haloalkyl, including C₁-C₆ haloalkyl, haloalkoxy, including C₁-C₆ haloalkoxy, halocycloalkyl, including C₃-C₈ halocycloalkyl, halocycloalkoxy, including C₃-C₈ halocycloalkoxy, amino, C₁-C₆ alkylamino, (C₁-C₆ alkyl)(C₁-C₆ alkyl)amino, alkylcarbonylamino, N—(C₁-C₆ alkyl)alkylcarbonylamino, aminoalkyl, C₁-C₆ alkylaminoalkyl, (C₁-C₆ alkyl)(C₁-C₆ alkyl)aminoalkyl, alkylcarbonylaminoalkyl, N—(C₁-C₆ alkyl)alkylcarbonylaminoalkyl, cyano, and nitro; or Z_{X} is selected from —CO₂R₄ and —CONR₅R₆, where R₄, R₅, and R₆ are each independently selected in each occurrence from hydrogen, C₁-C₆ alkyl, aryl-C₁-C₆ alkyl, and heteroaryl-C₁-C₆ alkyl.

As used herein, the term “leaving group” refers to a reactive functional group that generates an electrophilic site on the atom to which it is attached such that nucleophiles may be added to the electrophilic site on the atom. Illustrative leaving groups include, but are not limited to, halogens, optionally substituted phenols, acyloxy groups, sulfonium groups, sulfonoxy groups, and the like. Such leaving groups may be on alkyl, acyl, and the like. Such leaving groups may also be referred to herein as activating groups, such as when the leaving group is present on acyl. In addition, conventional peptide, amide, and ester coupling agents, such as but not limited to PyBop, BOP-Cl, BOP, pentafluorophenol, isobutylchloroformate, and the like, form various intermediates that include a leaving group, as defined herein, on a carbonyl group.

The recitation of a range of integers for any variable describes the recited range, every individual member in the range, and every possible subrange for that variable. For example, the recitation that n is an integer from 0 to 8, describes that range, the individual and selectable values of 0, 1, 2, 3, 4, 5, 6, 7, and 8, such as n is 0, or n is 1, or n is 2, etc. In addition, the recitation that n is an integer from 0 to 8 also describes each and every subrange, each of which may for the basis of a further embodiment, such as n is an integer from 1 to 8, from 1 to 7, from 1 to 6, from 2 to 8, from 2 to 7, from 1 to 3, from 2 to 4, etc.

The terms “treating”, “contacting” and “reacting” when referring to a chemical reaction generally mean to add or mix two or more reagents under appropriate conditions that allows a chemical transformation or chemical reaction to take place, and/or to produce the indicated and/or the desired product. The reaction, which produces the indicated and/or the desired product, may not necessarily result directly from the combination of two reagents which were initially added. In other words, there may be one or more intermediates which are produced in the mixture which ultimately leads to the formation of the indicated and/or the desired product.

The term “protecting group” general refers to any radical that is reversibly bonded to a functional group and is used to block or partially block the reactivity of that functional group to a predetermined set of conditions, such as reaction conditions. Illustratively, nitrogen protecting groups are reversibly bonded to amines to block or partially block the reactivity of the amine under a predetermined set of conditions. Illustrative nitrogen protecting groups include, but are not limited to, carbamates, such as t-Boc, Fmoc, and the like.

The term “composition” generally refers to any product comprising the specified ingredients, as well as any product which results, directly or indirectly, from combinations of the specified ingredients.

Certain functional groups, such as the hydroxy, amino, and like groups form complexes and/or coordination compounds with water and/or various solvents, in the various physical forms of the compounds. The compositions described herein may be prepared from isolated compounds or from salts, solutions, hydrates, solvates, and other forms of the compounds. The compositions may be prepared from various amorphous, non-amorphous, partially crystalline, crystalline, and/or other morphological forms of the compounds. The compositions may be prepared from various hydrates and/or solvates of the compounds. In addition, the compositions may be prepared from various co-crystals of the compounds. Accordingly, such pharmaceutical compositions that recite compounds are to be understood to include each of, or any combination of, the various morphological forms and/or solvate or hydrate forms of the compounds.

Illustratively, compositions may include one or more carriers, diluents, and/or excipients. The compounds, or compositions containing them, may be formulated in a therapeutically effective amount in any conventional dosage forms appropriate for the methods. The compounds, or compositions containing them, including such formulations, may be administered by a wide variety of conventional routes for the methods, and in a wide variety of dosage formats, utilizing known procedures (see generally, Remington: The Science and Practice of Pharmacy, (23_{rd} ed., 2020)).

The compounds can be used for both human clinical medicine and veterinary applications. Thus, the host animal with cancer and treated with the compounds can be human or, in the case of veterinary applications, can be a laboratory, agricultural, domestic, or wild animal. The disclosure can be applied to host animals including, but not limited to, humans, laboratory animals such rodents (e.g., mice, rats, hamsters, etc.), rabbits, monkeys, chimpanzees, domestic animals such as dogs, cats, and rabbits, agricultural animals such as cows, horses, pigs, sheep, goats, and wild animals in captivity such as bears, pandas, lions, tigers, leopards, elephants, zebras, giraffes, gorillas, dolphins, and whales.

The method can be utilized to treat such cancers as carcinomas, sarcomas, lymphomas, Hodgekin's disease, melanomas, mesotheliomas, Burkitt's lymphoma, nasopharyngeal carcinomas, leukemias, and myelomas. The cancer cell population can include, but is not limited to, oral, thyroid, endocrine, skin, gastric, esophageal, laryngeal, pancreatic, colon, bladder, bone, ovarian, cervical, uterine, breast, testicular, prostate, rectal, kidney, liver, and lung cancers.

The term “therapeutically effective amount” refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, the total daily usage of the compounds and compositions may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient; the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

The therapeutically effective amount, whether referring to monotherapy or combination therapy, is advantageously selected with reference to any toxicity, or other undesirable side effect, that might occur during administration of one or more of the compounds described herein. Further, the co-therapies may allow for the administration of lower doses of compounds that show such toxicity, or other undesirable side effect, where those lower doses are below thresholds of toxicity or lower in the therapeutic window than would otherwise be administered in the absence of a co-therapy.

An effective amount of any one or a mixture of the compounds described herein can be readily determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

In the methods, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

In addition, in those embodiments drawn to combination therapy comprising administration of a compound of formula (I) and another anticancer drug known in the art, “therapeutically effective amount” refers to that amount of the combination of agents taken together so that the combined effect elicits the desired biological or medicinal response. For example, the therapeutically effective amount of a compound of formula (I) and imatinib, and the like, would be the amount of a compound of formula (I) and the amount of imatinib, and the like that when taken together or sequentially have a combined effect that is therapeutically effective. Further, in some embodiments of such methods that include coadministration, that coadministration amount of a compound of formula (I) or imatinib, and the like when taken individually may or may not be therapeutically effective.

The term “administering” includes all means of introducing the compounds and compositions to the host animal, including, but are not limited to, oral (po), intravenous (iv), intramuscular (im), subcutaneous (sc), transdermal, inhalation, buccal, ocular, sublingual, vaginal, rectal, and the like. The compounds and compositions may be administered in unit dosage forms and/or formulations containing conventional nontoxic pharmaceutically acceptable carriers, adjuvants, and/or vehicles.

Illustrative formats for oral administration include tablets, capsules, elixirs, syrups, and the like.

Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound is not sufficiently soluble to be dissolved, a solubilizer such as ethanol can be applied.

Illustratively, administering includes local use, such as when administered locally to the site of disease, or to a particular organ or tissue system. Illustrative local administration may be performed during open surgery, or other procedures when the site of disease is accessible. Alternatively, local administration may be performed using parenteral delivery where the compound or compositions are deposited locally to the site without general distribution to multiple other non-target sites in the host animal being treated. Similar variations regarding local delivery to particular tissue types, such as organs, and the like, are also described. Illustratively, compounds may be administered directly to the nervous system including, but not limited to, intracerebral, intraventricular, intracerebroventricular, intrathecal, intracisternal, intraspinal and/or peri-spinal routes of administration by delivery via intracranial or intravertebral needles and/or catheters with or without pump devices.

In making the pharmaceutical compositions of the compounds, a therapeutically effective amount of one or more compounds in any of the various forms described herein may be mixed with one or more excipients, diluted by one or more excipients, or enclosed within such a carrier which can be in the form of a capsule, sachet, paper, or other container. Excipients may serve as a diluent, and can be solid, semi-solid, or liquid materials, which act as a vehicle, carrier or medium for the active ingredient. Thus, the formulation compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders. The compositions may contain anywhere from about 0.1% to about 99.9% active ingredients, depending upon the selected dose and dosage form.

Illustrative examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include lubricating agents such as talc, magnesium stearate, and mineral oil; wetting agents; emulsifying and suspending agents; preserving agents such as methyl- and propylhydroxybenzoates; sweetening agents; and flavoring agents. The compositions can be formulated to provide quick, sustained or delayed release of the active ingredient after administration to the host animal by employing procedures known in the art. One or more carriers, one or more diluents, one or more excipients, and combinations of the foregoing may be used in making the pharmaceutical compositions. It is appreciated that the carriers, diluents, and excipients used to prepare the compositions described herein are advantageously GRAS (generally regarded as safe) compounds. It is also appreciated that acids and bases used to make salts, as described herein, and/or solvents used to make solvates, as described herein, are also advantageously GRAS compounds.

Illustrative examples of emulsifying agents include naturally occurring gums (e.g., gum acacia or gum tragacanth) and naturally occurring phosphatides (e.g., soybean lecithin and sorbitan monooleate derivatives). Examples of antioxidants are butylated hydroxy anisole (BHA), ascorbic acid and derivatives thereof, tocopherol and derivatives thereof, butylated hydroxy anisole, and cysteine. Examples of preservatives are parabens, such as methyl or propyl p-hydroxybenzoate, and benzalkonium chloride. Examples of humectants are glycerin, propylene glycol, sorbitol, and urea. Examples of penetration enhancers are propylene glycol, DMSO, triethanolamine, N,N-dimethylacetamide, N,N-dimethylformamide, 2-pyrrolidone and derivatives thereof, tetrahydrofurfuryl alcohol, and AZONE. Examples of chelating agents are sodium EDTA, citric acid, and phosphoric acid. Examples of gel forming agents are CARBOPOL, cellulose derivatives, bentonite, alginates, gelatin and polyvinylpyrrolidone. Examples of ointment bases are beeswax, paraffin, cetyl palmitate, vegetable oils, sorbitan esters of fatty acids (Span), polyethylene glycols, and condensation products between sorbitan esters of fatty acids and ethylene oxide (e.g., polyoxyethylene sorbitan monooleate (TWEEN)).

Therapeutically effect doses administered in animal models may be used to calculate corresponding therapeutically effect doses for administration to other host animals, including humans. Illustrative corresponding doses may be calculated using the “Guidance for Industry Estimating the Maximum Safe Starting Dose in Initial Clinical Trials for Therapeutics in Adult Healthy Volunteers,” which is published by the FDA can be found at https://www.fda.gov/media/72309/download, and is incorporated herein in its entirety by reference.

### Compounds of Formula (I)

In an embodiment, the present disclosure provides compounds of formula (I), or salts, hydrates or solvates thereof:

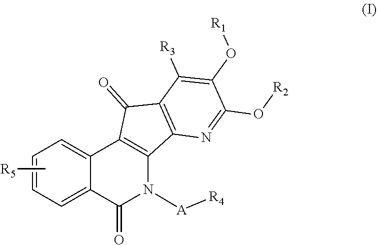

wherein:
R₁ and R₂ are independently selected from the group consisting of hydrogen, (C₁-C₆) alkyl, (C₂-C₆)alkenyl, (C₂-C₆)alkynyl, optionally substituted heteroalkyl, and optionally substituted acyl, or R₁ and R₂ together with the atoms to which they are attached form a 5-membered or 6-membered ring;
R₃ is hydrogen, halo, nitro, cyan, CF₃, (C₁-C₆)alkyl, (C₁-C₆)alkylthio, or (C₁-C₆)alkoxy;
A is alkylene;
R₄ is selected from the group consisting of heteroaryl, heterocyclyl, heterocyclylamino, amino, hydroxyl, halo, cyano, alkylamino, dialkylamino, hydroxyalkylamino, bis(hydroxyalkyl)amino, and hydroxyalkylaminoalkylamino, wherein each of heteroaryl, heterocyclyl, and heterocyclylamino is optionally substituted;
R₅ represents from 1 to 2 substituents independently selected from the group consisting of amino, (C₁-C₆)alkylamino, di(C₁-C₆)alkylamino, hydroxy (C₁-C₆)alkyl, (C₁-C₆)alkenyl, (C₁-C₆)alkynyl, (C₁-C₆)heteroalkyl, (C₃-C₈)cycloalkyl, (C₃-C₈)cycloheteroalkyl, (C₁-C₆)alkoxy, (C₁-C₆)alkyl(CO)O—, (C₁-C₆)alkyl-O(CO)O— and (C₁-C₆)alkylthio; or R₅ represents 2 adjacent substituents that are taken together with the attached carbons to form an optionally substituted cycle or heterocycle.

In an illustrative embodiment, compounds of formula (I), or salts, hydrates or solvates thereof, are described:

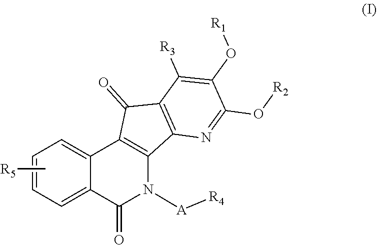

wherein A is (CH₂)n, where n is 1, 2, or 3;
R₁ and R₂ are independently selected in each instance from the group consisting of hydrogen, (C₁-C₆) alkyl, (C₂-C₆)alkenyl, (C₂-C₆)alkynyl, optionally substituted heteroalkyl, and optionally substituted acyl, or R₁ and R₂ together with the atoms to which they are attached form a 5-membered or 6-membered ring;
R₃ is hydrogen, halo, nitro, cyan, CF₃, (C₁-C₆)alkyl, (C₁-C₆)alkylthio, or (C₁-C₆)alkoxy;
R₄ is selected from the group consisting of heteroaryl, heterocyclyl, heterocyclylamino, amino, hydroxyl, halo, cyano, alkylamino, dialkylamino, hydroxyalkylamino, bis(hydroxyalkyl)amino, and hydroxyalkylaminoalkylamino, wherein each of heteroaryl, heterocyclyl, and heterocyclylamino is optionally substituted;
R₅ represents from 1 to 2 substituents independently selected in each instance from the group consisting of amino, (C₁-C₆)alkylamino, di(C₁-C₆)alkylamino, hydroxy (C₁—C₆)alkyl, (C₁-C₆)alkenyl, (C₁-C₆)alkynyl, (C₁-C₆)heteroalkyl, (C₃-C₈)cycloalkyl, (C₃-C₈)cycloheteroalkyl, (C₁-C₆)alkoxy, (C₁-C₆)alkyl(CO)O—, (C₁-C₆)alkyl-O(CO)O— and (C₁-C₆)alkylthio; or R₅ represents 2 adjacent substituents that are taken together with the attached carbons to form an optionally substituted cycle or heterocycle.

In another illustrative embodiment, compounds of formula (I), or salts, hydrates, or solvates thereof, are described wherein n is an integer from 1 to 3, or n is an integer from 2 to 3, or n is 3.

In another illustrative embodiment, compounds of formula (I), or salts, hydrates, or solvates thereof, are described wherein A is (CH₂)₃.

In another illustrative embodiment, compounds of formula (I), or salts, hydrates, or solvates thereof, are described wherein R₃ is hydrogen.

In another illustrative embodiment, compounds of formula (I), or salts, hydrates, or solvates thereof, are described wherein R₁ and R₂ are independently hydrogen or C₁-C₆ alkyl.

In another illustrative embodiment, compounds of formula (I), or salts, hydrates, or solvates thereof, are described wherein R₁ and R₂ are independently hydrogen or CH₃.

In another illustrative embodiment, compounds of formula (I), or salts, hydrates, or solvates thereof, are described wherein R₁ and R₂ are CH₃.

In another illustrative embodiment, compounds of formula (I), or salts, hydrates, or solvates thereof, are described wherein R₁ and R₂ taken together with the atoms they are attached to form a cycle.

In another illustrative embodiment, compounds of formula (I), or salts, hydrates, or solvates thereof, are described wherein R₁ and R₂ are taken together to form —CH₂—.

In another illustrative embodiment, compounds of formula (I), or salts, hydrates, or solvates thereof, are described wherein R₃ is hydrogen, (C₁-C₆)alkyl, (C₁-C₆)alkylthio, or (C₁-C₆)alkoxy.

In another illustrative embodiment, compounds of formula (I), or salts, hydrates, or solvates thereof, are described wherein R₃ is hydrogen.

In another illustrative embodiment, compounds of formula (I), or salts, hydrates, or solvates thereof, are described wherein R₄ is selected from the group consisting of heteroaryl, heterocyclyl, heterocyclylamino, amino, hydroxyl, halo, cyano, alkylamino, dialkylamino, hydroxyalkylamino, bis(hydroxyalkyl)amino, and hydroxyalkylaminoalkylamino, wherein each of heteroaryl, heterocyclyl, and heterocyclylamino is optionally substituted.

In another illustrative embodiment, compounds of formula (I), or salts, hydrates, or solvates thereof, are described wherein R₄ is selected from the group consisting of

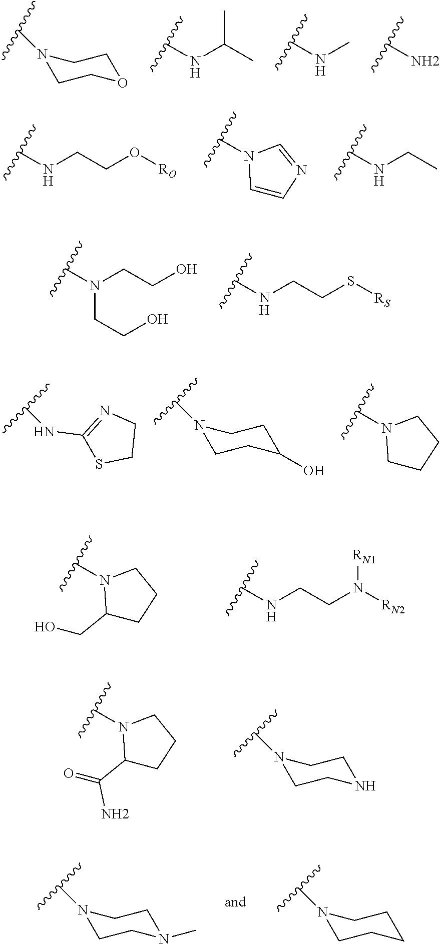

wherein R_{O}, R_{S}, R_{N1}, and R_{N2} are in each instance independently selected from hydrogen and optionally substituted (C₁-C₆)alkyl or R_{N1} and R_{N2} are taken together with the attached nitrogen to form an optionally substituted heterocycle.

In another illustrative embodiment, compounds of formula (I), or salts, hydrates, or solvates thereof, are described wherein R₄ is selected from the group consisting of

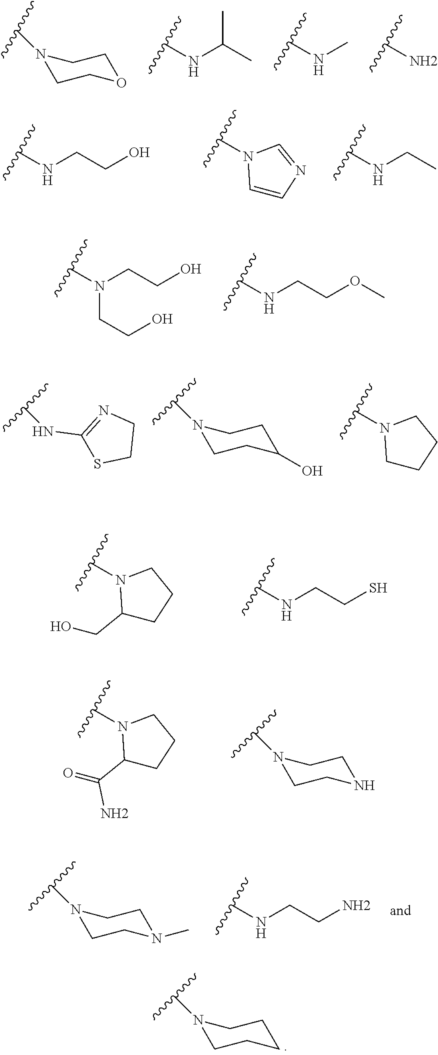

In another illustrative embodiment, compounds of formula (I), or salts, hydrates, or solvates thereof are described wherein R₁ and R₂ are taken together to form —CH₂—, R₃ is hydrogen, R₅ represents 2-MeO and 3-MeO, A is (CH₂)₃, and R₄ is selected from the group consisting of heteroaryl, heteroaryloxy, heteroarylamino, heteroarylalkylaminoalkylamino, heterocyclyl, heterocyclylamino, amino, hydroxyl, halo, cyano, alkylamino, dialkylamino, trialkylammonium, hydroxyalkylamino, bis(hydroxyalkyl)amino, and hydroxyalkylaminoalkylamino, wherein each of heteroaryl, heteroaryloxy, and heteroarylamino, heteroarylalkylaminoalkylamino, heterocyclyl, and heterocyclylamino is optionally substituted.

In another illustrative embodiment, compounds of formula (I), or salts, hydrates, or solvates thereof are described wherein R₁ and R₂ are taken together to form —CH₂—, R₃ is hydrogen, R₅ represents 2-MeO and 3-MeO, A is (CH₂)₃, and R₄ is selected from the group consisting of

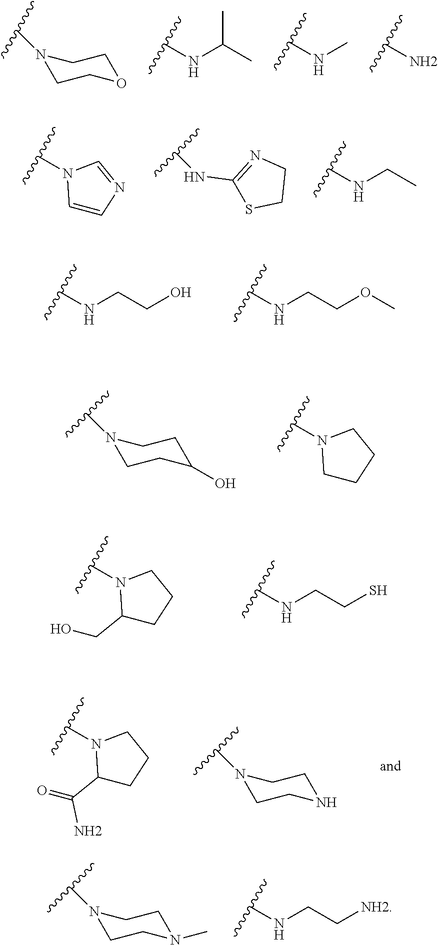

In another illustrative embodiment, compounds of formula (I), or salts, hydrates, or solvates thereof are described wherein R₁ and R₂ are taken together to form —CH₂—, R₃ is hydrogen, R₅ represents 2-MeO and 3-MeO, A is (CH₂)₃, and R₄ is selected from the group consisting of

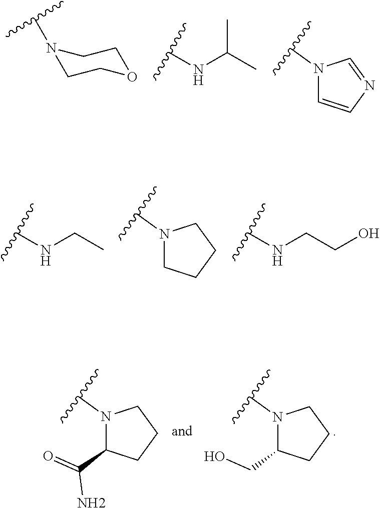

In another illustrative embodiment, a compound of the following formula, or a salt, hydrate, or solvate thereof, is described

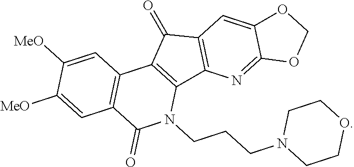 [SMILES: COc1cc2c3c(n(CCCN4CCOCC4)c(=O)c2cc1OC)-c1nc2c(cc1C3=O)OCO2]

In another illustrative embodiment, a compound of the following formula, or a salt, hydrate, or solvate thereof, is described

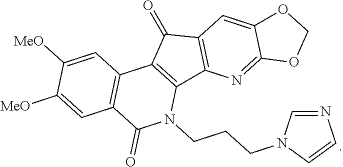 [SMILES: COc1cc2c3c(n(CCCn4ccnc4)c(=O)c2cc1OC)-c1nc2c(cc1C3=O)OCO2]

In another illustrative embodiment, a compound of the following formula, or a salt, hydrate, or solvate thereof, is described

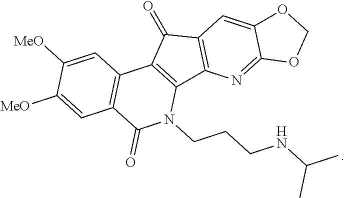 [SMILES: COc1cc2c3c(n(CCCNC(C)C)c(=O)c2cc1OC)-c1nc2c(cc1C3=O)OCO2]

In another illustrative embodiment, a compound of the following formula, or a salt, hydrate, or solvate thereof, is described

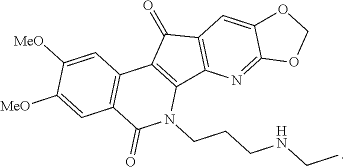 [SMILES: CCNCCCn1c2c(c3cc(OC)c(OC)cc3c1=O)C(=O)c1cc3c(nc1-2)OCO3]

In another illustrative embodiment, a compound of the following formula, or a salt, hydrate, or solvate thereof, is described

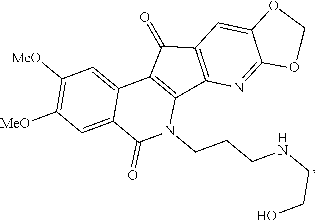 [SMILES: COc1cc2c3c(n(CCCNCCO)c(=O)c2cc1OC)-c1nc2c(cc1C3=O)OCO2]

In another illustrative embodiment, a compound of the following formula, or a salt, hydrate, or solvate thereof, is described

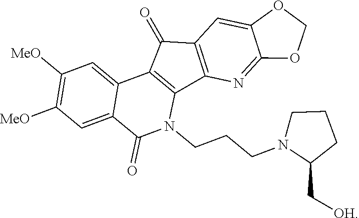 [SMILES: COc1cc2c3c(n(CCCN4CCC[C@H]4CO)c(=O)c2cc1OC)-c1nc2c(cc1C3=O)OCO2]

In another illustrative embodiment, a compound of the following formula, or a salt, hydrate, or solvate thereof, is described

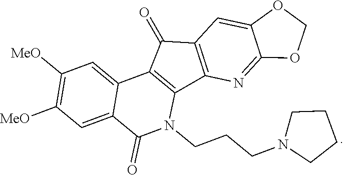 [SMILES: COc1cc2c3c(n(CCCN4CCCC4)c(=O)c2cc1OC)-c1nc2c(cc1C3=O)OCO2]

In another illustrative embodiment, a compound of the following formula, or a salt, hydrate, or solvate thereof, is described

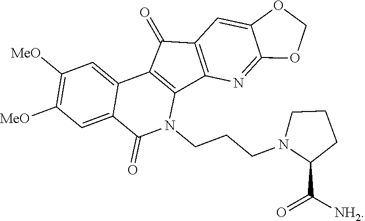 [SMILES: COc1cc2c3c(n(CCCN4CCC[C@H]4C(N)=O)c(=O)c2cc1OC)-c1nc2c(cc1C3=O)OCO2]

It is to be understood that all possible combinations of the various genera and subgenera of each of A, R₁, R₂, R₃, R₄, R₅, R_{O}, R_{S}, R_{N1}, and R_{N2} described represent additional illustrative embodiments of compounds. It is to be further understood that each of those additional illustrative embodiments of compounds may be used in any of the compositions, methods, unit doses, kits, and/or uses described.

In an embodiment, the disclosure provides a pharmaceutical composition containing one or more compounds of formula (I), or a salt, hydrate or solvate thereof. In an embodiment, the pharmaceutical composition includes a therapeutically effective amount of the one or more of the compounds for treating a host animal with a cancer. It is to be understood that the compositions may include other components and/or ingredients, including, but not limited to, other therapeutically active compounds, and/or one or more carriers, vehicles, diluents, adjuvants, excipients, and the like, and combinations thereof.

In another embodiment, unit doses of one or more compounds of formula (I) and pharmaceutical compositions containing one or more of the compounds of formula (I) are also described herein. The unit doses include a therapeutically effective amount of the one or more of the compounds of formula (I) for treating a host animal with cancer. The unit doses are in single or divided form, and may correspond to a daily dosage amount, or adjusted to a periodic amount that is shorter, including for multiple daily doses, or longer, including weekly or monthly doses. The compositions may include other components and/or ingredients, including, but not limited to, other therapeutically active compounds, and/or one or more carriers, vehicles, diluents, adjuvants, excipients, and the like, and combinations thereof.

In another embodiment, methods for treating host animals with cancer are also described, where the methods include administering one or more of the compounds of formula (I), or salts, hydrates or solvates thereof, and/or compositions to a host animal with cancer. In another embodiment, the methods include administering a therapeutically effective amount of the one or more compounds of formula (I) and/or compositions for treating host animals with cancer.

In another illustrative embodiment, a method for treating a host animal with cancer is described wherein the method comprises administering to the host animal one or more compounds of formula (I), or a salt, hydrate or solvate thereof,

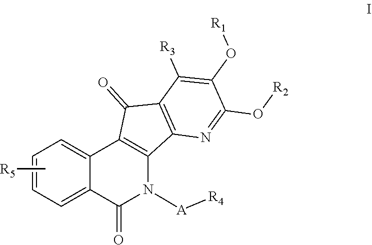

wherein R₁ and R₂ are independently selected from the group consisting of hydrogen, (C₁-C₆) alkyl, (C₂-C₆)alkenyl, (C₂-C₆)alkynyl, optionally substituted heteroalkyl, and optionally substituted acyl, or R₁ and R₂ together with the atoms to which they are attached form a 5-membered or 6-membered ring;
R₃ is hydrogen, halo, nitro, cyan, CF₃, (C₁-C₆)alkyl, (C₁-C₆)alkylthio, or (C₁-C₆)alkoxy;
A is alkylene;
R₄ is selected from the group consisting of heteroaryl, heterocyclyl, heterocyclylamino, amino, hydroxyl, halo, cyano, alkylamino, dialkylamino, hydroxyalkylamino, bis(hydroxyalkyl)amino, and hydroxyalkylaminoalkylamino, wherein each of heteroaryl, heterocyclyl, and heterocyclylamino is optionally substituted;
R₅ represents from 1 to 2 substituents independently selected in each instance from the group consisting of amino, (C₁-C₆)alkylamino, di(C₁-C₆)alkylamino, hydroxy (C₁-C₆)alkyl, (C₁-C₆)alkenyl, (C₁-C₆)alkynyl, (C₁-C₆)heteroalkyl, (C₃-C₈)cycloalkyl, (C₃-C₈)cycloheteroalkyl, (C₁-C₆)alkoxy, (C₁-C₆)alkyl(CO)O—, (C₁-C₆)alkyl-O(CO)O— and (C₁-C₆)alkylthio; or R₅ represents 2 adjacent substituents that are taken together with the attached carbons to form an optionally substituted cycle or heterocycle.

In another illustrative embodiment, the method for treating the host animal with cancer comprises administering to the host animal one or more compounds of formula (I), or salts, hydrates, or solvates thereof, or a composition comprising one or more compounds of formula (I), or salts, hydrates or solvates thereof, wherein
A is (CH₂)n, where n is 1, 2, or 3;
R₁ and R₂ are independently selected in each instance from the group consisting of hydrogen, (C₁-C₆) alkyl, (C₂-C₆)alkenyl, (C₂-C₆)alkynyl, optionally substituted heteroalkyl, and optionally substituted acyl, or R₁ and R₂ together with the atoms to which they are attached form a 5-membered or 6-membered ring;
R₃ is hydrogen, halo, nitro, cyan, CF₃, (C₁-C₆)alkyl, (C₁-C₆)alkylthio, or (C₁-C₆)alkoxy;
R₄ is selected from the group consisting of heteroaryl, heterocyclyl, heterocyclylamino, amino, hydroxyl, halo, cyano, alkylamino, dialkylamino, hydroxyalkylamino, bis(hydroxyalkyl)amino, and hydroxyalkylaminoalkylamino, wherein each of heteroaryl, heterocyclyl, and heterocyclylamino is optionally substituted;
R₅ represents from 1 to 2 substituents independently selected in each instance from the group consisting of amino, (C₁-C₆)alkylamino, di(C₁-C₆)alkylamino, hydroxy (C₁-C₆)alkyl, (C₁-C₆)alkenyl, (C₁-C₆)alkynyl, (C₁-C₆)heteroalkyl, (C₃-C₈)cycloalkyl, (C₃-C₈)cycloheteroalkyl, (C₁-C₆)alkoxy, (C₁-C₆)alkyl(CO)O—, (C₁-C₆)alkyl-O(CO)O— and (C₁-C₆)alkylthio; or R₅ represents 2 adjacent substituents that are taken together with the attached carbons to form an optionally substituted cycle or heterocycle.

In another illustrative embodiment, the method for treating the host animal with cancer comprises administering to the host animal one or more compounds of formula (I), or salts, hydrates, or solvates thereof, or a composition comprising one or more compounds of formula (I), or salts, hydrates or solvates thereof, wherein A is (CH₂)n, where n is an integer from 1 to 3, or n is an integer from 2 to 3, or n is 3.

In another illustrative embodiment, the method for treating the host animal with cancer comprises administering to the host animal one or more compounds of formula (I), or salts, hydrates, or solvates thereof, or a composition comprising one or more compounds of formula (I), or salts, hydrates or solvates thereof, wherein R₁ and R₂ are independently hydrogen or C₁-C₆ alkyl.

In another illustrative embodiment, the method for treating the host animal with cancer comprises administering to the host animal one or more compounds of formula (I), or salts, hydrates, or solvates thereof, or a composition comprising one or more compounds of formula (I), or salts, hydrates or solvates thereof, wherein R₁ and R₂ are independently hydrogen or CH₃.

In another illustrative embodiment, the method for treating the host animal with cancer comprises administering to the host animal one or more compounds of formula (I), or salts, hydrates, or solvates thereof, or a composition comprising one or more compounds of formula (I), or salts, hydrates or solvates thereof, wherein R₁ and R₂ are CH₃.

In another illustrative embodiment, the method for treating the host animal with cancer comprises administering to the host animal one or more compounds of formula (I), or salts, hydrates, or solvates thereof, or a composition comprising one or more compounds of formula (I), or salts, hydrates or solvates thereof, wherein R₁ and R₂ taken together with the atoms they are attached to form a cycle.

In another illustrative embodiment, the method for treating the host animal with cancer comprises administering to the host animal one or more compounds of formula (I), or salts, hydrates, or solvates thereof, or a composition comprising one or more compounds of formula (I), or salts, hydrates or solvates thereof, wherein R₁ and R₂ are taken together to form —CH₂—.

In another illustrative embodiment, the method for treating the host animal with cancer comprises administering to the host animal one or more compounds of formula (I), or salts, hydrates, or solvates thereof, or a composition comprising one or more compounds of formula (I), or salts, hydrates or solvates thereof, wherein R₃ is hydrogen, (C₁-C₆)alkyl, (C₁-C₆)alkylthio, or (C₁-C₆)alkoxy.

In another illustrative embodiment, the method for treating the host animal with cancer comprises administering to the host animal one or more compounds of formula (I), or salts, hydrates, or solvates thereof, or a composition comprising one or more compounds of formula (I), or salts, hydrates or solvates thereof wherein R₃ is hydrogen.

In another illustrative embodiment, the method for treating the host animal with cancer comprises administering to the host animal one or more compounds of formula (I), or salts, hydrates, or solvates thereof, or a composition comprising one or more compounds of formula (I), or salts, hydrates or solvates thereof, wherein R₄ is selected from the group consisting of heteroaryl, heterocyclyl, heterocyclylamino, amino, hydroxyl, halo, cyano, alkylamino, dialkylamino, hydroxyalkylamino, bis(hydroxyalkyl)amino, and hydroxyalkylaminoalkylamino, wherein each of heteroaryl, heterocyclyl, and heterocyclylamino is optionally substituted.

In another illustrative embodiment, the method for treating the host animal with cancer comprises administering to the host animal one or more compounds of formula (I), or salts, hydrates, or solvates thereof, or a composition comprising one or more compounds of formula (I), or salts, hydrates or solvates thereof, wherein R₄ is selected from the group consisting of

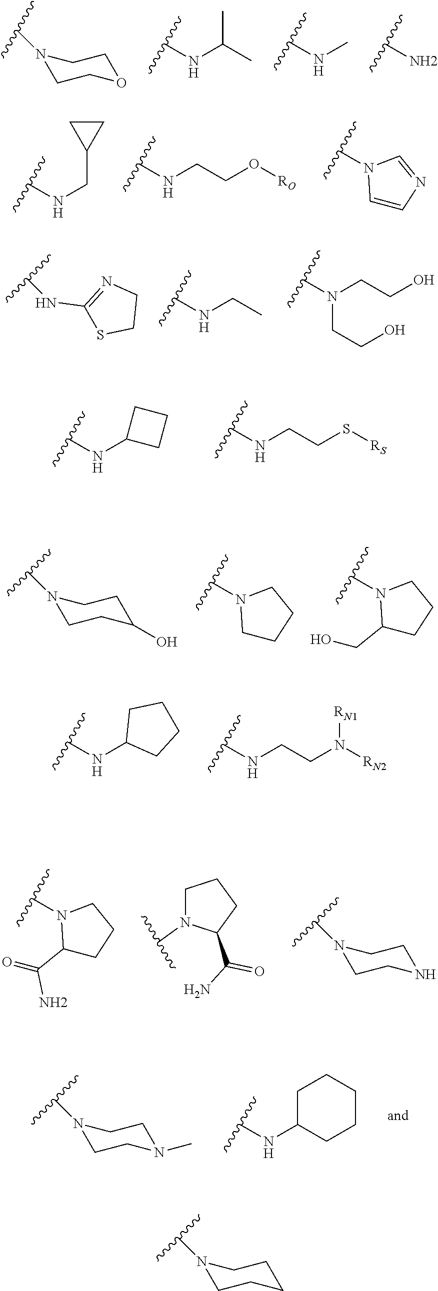

where R_{O}, R_{S}, R_{N1}, and R_{N2} are in each instance independently selected from hydrogen and optionally substituted (C₁-C₆)alkyl or R_{N1} and R_{N2} are taken together with the attached nitrogen to form an optionally substituted heterocycle.

In another illustrative embodiment, the method for treating the host animal with cancer comprises administering to the host animal one or more compounds of formula (I), or salts, hydrates, or solvates thereof, or a composition comprising one or more compounds of formula (I), or salts, hydrates or solvates thereof, wherein R₄ is selected from the group consisting of

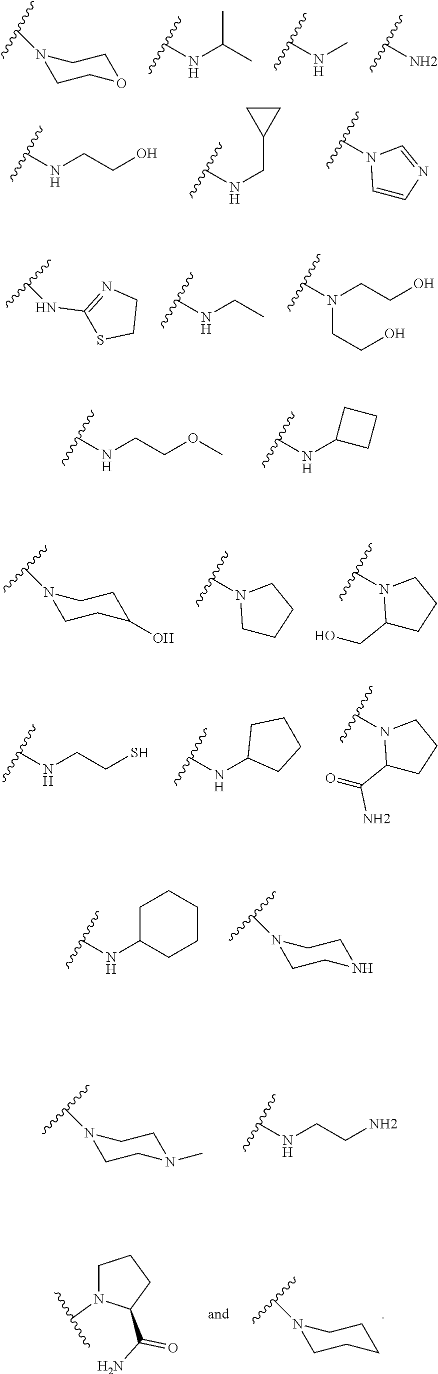

In another illustrative embodiment, the method for treating the host animal with cancer comprises administering to the host animal one or more compounds of formula (I), or a salt, hydrate, or solvate thereof, or a composition comprising one or more compounds of formula (I) or a salt, hydrate or solvate thereof wherein R₁ and R₂ are taken together to form —CH₂—, R₃ is hydrogen, R₅ represents 2-MeO and 3-MeO, A is (CH₂)₃, and R₄ is selected from the group consisting of heteroaryl, heteroaryloxy, heteroarylamino, heteroarylalkylaminoalkylamino, heterocyclyl, heterocyclylamino, amino, hydroxyl, halo, cyano, alkylamino, dialkylamino, trialkylammonium, hydroxyalkylamino, bis(hydroxyalkyl)amino, and hydroxyalkylaminoalkylamino, wherein each of heteroaryl, heteroaryloxy, and heteroarylamino, heteroarylalkylaminoalkylamino, heterocyclyl, and heterocyclylamino is optionally substituted.

In another illustrative embodiment, the method for treating the host animal with cancer comprises administering to the host animal one or more compounds of formula (I) or salts, hydrates, or solvates thereof, or a composition comprising one or more compounds of formula (I), or salts, hydrates or solvates thereof, wherein R₁ and R₂ are taken together to form —CH₂—, R₃ is hydrogen, R₅ represents 2-MeO and 3-MeO, A is (CH₂)₃, and R₄ is selected from the group consisting of

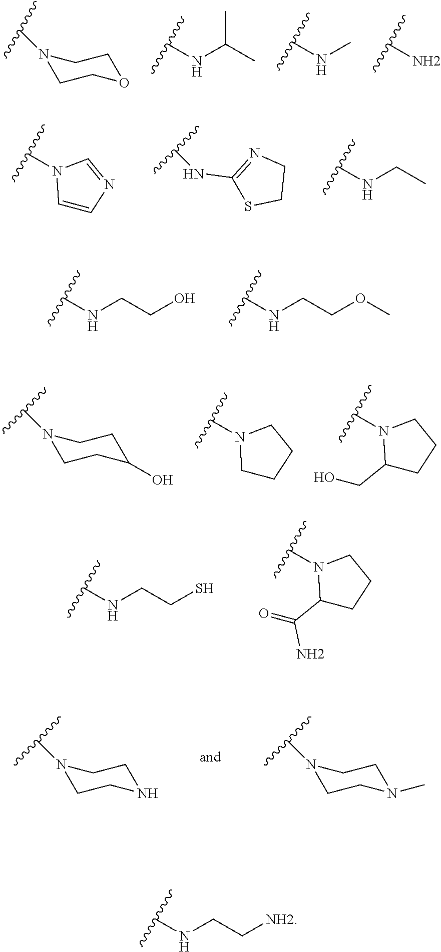

In another illustrative embodiment, the method for treating the host animal with cancer comprises administering to the host animal one or more compounds of formula (I), or salts, hydrates, or solvates thereof, or a composition comprising one or more compounds of formula (I), or salts, hydrates or solvates thereof, wherein R₁ and R₂ are taken together to form —CH₂—, R₃ is hydrogen, R₅ represents 2-MeO and 3-MeO, A is (CH₂)₃, and R₄ is selected from the group consisting of

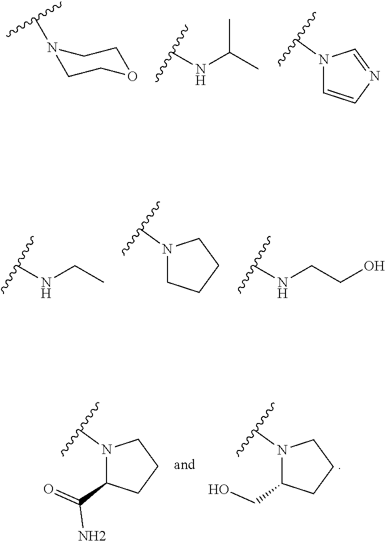

In another illustrative embodiment, the method for treating the host animal with cancer comprises administering to the host animal the following compound (G101)

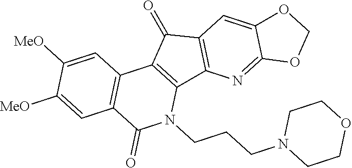 [SMILES: COc1cc2c3c(n(CCCN4CCOCC4)c(=O)c2cc1OC)-c1nc2c(cc1C3=O)OCO2]

or a salt, hydrate, or solvate thereof, or a composition comprising the compound or a salt, hydrate or solvate thereof.

In another illustrative embodiment, the method for treating the host animal with cancer comprises administering to the host animal the following compound (G102)

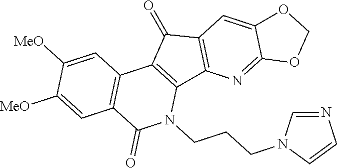 [SMILES: COc1cc2c3c(n(CCCn4ccnc4)c(=O)c2cc1OC)-c1nc2c(cc1C3=O)OCO2]

or a salt, hydrate, or solvate thereof, or a composition comprising the compound or a salt, hydrate or solvate thereof.

In another illustrative embodiment, the method for treating the host animal with cancer comprises administering to the host animal the following compound (G103)

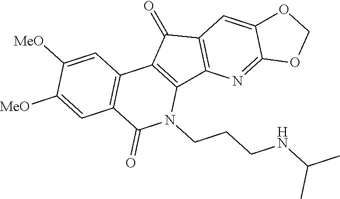 [SMILES: COc1cc2c3c(n(CCCNC(C)C)c(=O)c2cc1OC)-c1nc2c(cc1C3=O)OCO2]

or a salt, hydrate, or solvate thereof, or a composition comprising the compound or a salt, hydrate or solvate thereof.

In another illustrative embodiment, the method for treating the host animal with cancer comprises administering to the host animal the following compound (G104)

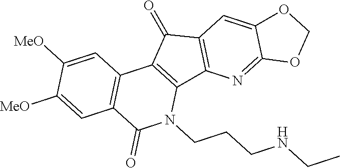 [SMILES: CCNCCCn1c2c(c3cc(OC)c(OC)cc3c1=O)C(=O)c1cc3c(nc1-2)OCO3]

or a salt, hydrate, or solvate thereof, or a composition comprising the compound or a salt, hydrate or solvate thereof.

In another illustrative embodiment, the method for treating the host animal with cancer comprises administering to the host animal the following compound (G105)

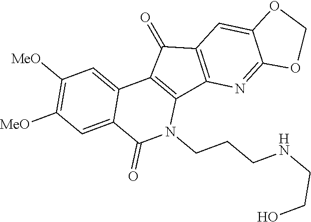 [SMILES: COc1cc2c3c(n(CCCNCCO)c(=O)c2cc1OC)-c1nc2c(cc1C3=O)OCO2]

or a salt, hydrate, or solvate thereof, or a composition comprising the compound or a salt, hydrate or solvate thereof.

In another illustrative embodiment, the method for treating the host animal with cancer comprises administering to the host animal the following compound (G108)

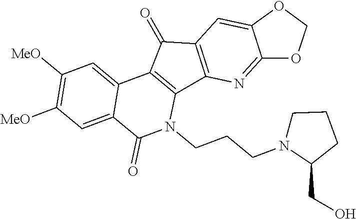 [SMILES: COc1cc2c3c(n(CCCN4CCC[C@H]4CO)c(=O)c2cc1OC)-c1nc2c(cc1C3=O)OCO2]

or a salt, hydrate, or solvate thereof, or a composition comprising the compound or a salt, hydrate or solvate thereof.

In another illustrative embodiment, the method for treating the host animal with cancer comprises administering to the host animal the following compound (G106)

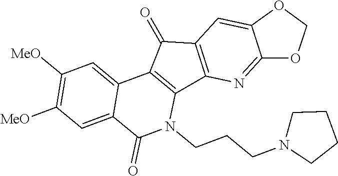 [SMILES: COc1cc2c3c(n(CCCN4CCCC4)c(=O)c2cc1OC)-c1nc2c(cc1C3=O)OCO2]

or a salt, hydrate, or solvate thereof, or a composition comprising the compound or a salt, hydrate or solvate thereof.

In another illustrative embodiment, the method for treating the host animal with cancer comprises administering to the host animal the following compound (G107)

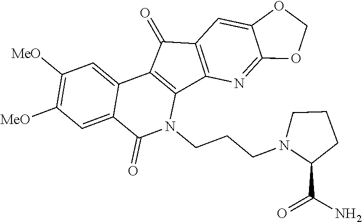 [SMILES: COc1cc2c3c(n(CCCN4CCC[C@H]4C(N)=O)c(=O)c2cc1OC)-c1nc2c(cc1C3=O)OCO2]

or a salt, hydrate, or solvate thereof, or a composition comprising the compound or a salt, hydrate or solvate thereof.

In another embodiment, uses of the compounds and compositions in the manufacture of a medicament for treating host animals with cancer are also described. In another embodiment, the medicaments include a therapeutically effective amount of the one or more compounds, or salts, hydrates or solvates thereof, and/or compositions comprising same for treating a host animal with cancer.

The compounds, compositions, unit doses, and methods may be used alone or in combination with other compounds useful for treating cancer, including those compounds that may be therapeutically effective by the same or different modes of action. In addition, the compounds may be used in combination with other compounds that are administered to treat other symptoms of cancer, such as compounds administered to cancer, and the like.

Topoisomerase I inhibitors are classified as being either suppressors, which inhibit the DNA cleavage reaction catalyzed by the enzyme, or poisons, which inhibit the DNA relegation reaction that is catalyzed by the enzyme after cleavage occurs (Scheme 1). Since the discovery that the indenoisoquinoline NSC314622 (1)¹ acts as a topoisomerase I poison by intercalating between the base pairs at the cleavage site,² several additional biological macromolecules have been found to be targeted by variously substituted indenoisoquinolines, including topoisomerase II,³⁻⁷ retinoid X receptor (RXR),⁸⁻¹⁰ PARP-1,¹¹ the estrogen receptor,¹² tyrosyl-DNA phosphodiesterase 1 (TDP1),¹³⁻¹⁷ and tyrosyl-DNA phosphodiesterase 2 (TDP2).¹³ More recently, an array of indenoisoquinolines were reported to stabilize the G-quadruplex in the MYC promoter and downregulate MYC protein expression through a mechanism involving end-stacking of the indenoisoquinolines with the external tetrads of the G-quadruplex.¹⁸ Three indenoisoquinoline topoisomerase 1 poisons, LMP400 (indotecan, 2),¹⁹ LMP776 (indimitecan, 3),¹⁹ and LMP744 (4),²⁰ have successfully completed or will soon complete phase 1 clinical trials,²¹ and notably, all three of those drugs were also found to be MYC promoter G-quadruplex stabilizers and downregulate MYC protein expression.¹⁸ Furthermore, the indenoisoquinolines may stabilize G-quadruplexes by two mechanisms: (1) inhibition of topoisomerase I to maintain negative DNA supercoiling associated with G-quadruplex formation, and (2) end-stacking with the two external G-quadruplex tetrads.¹⁸ In fact, the two activities (topoisomerase I inhibition and G-quadruplex stabilization) have been found to act synergistically with regard to cancer cell cytotoxicity.¹⁸ Herein are described the results of studies aimed at optimization of the indenoisoquinolines vs. both targets in an attempt to maximize their anticancer activities.

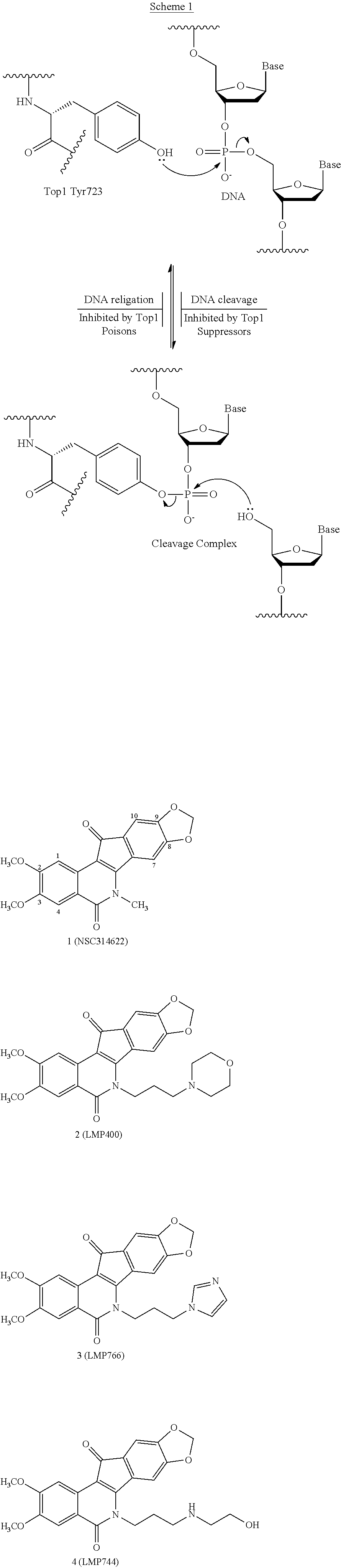

Previous attempts to optimize the anticancer activities of the indenoisoquinolines involved the instillation of single nitrogen atoms in all of the possible locations in the two aromatic rings. More specifically, carbons at the 1-4 and 7-10 positions were each replaced individually with nitrogen atoms one at a time.^{13, 22-24} The rationale was that the electronegative nitrogens would facilitate π-π stacking of the azaindenoisoquinolines with the neighboring base pairs in the ternary drug-DNA-topoisomerase I cleavage complex by facilitating charge transfer complex formation involving the base pairs as electron donors and the azaindenoisoquinolines as electron acceptors. These studies revealed the fact that the 7-aza analogues were the most cytotoxic in cancer cell cultures, especially when combined with a methoxyl substituent in the 9-position and a nitro, fluoro, or chloro substituent in the 3-position.^{13, 23-25} Quantum mechanics calculations of models of π-πstacking of the 7-, 8-, 9-, and 10-azaindenoisoquinolines with the neighboring DNA base pairs appears to confirm that the electronegative nitrogen atoms do in fact increase charge transfer complex formation. However, this effect appears to be offset in the 8-, 9-, and 10-azaindenoisoquinolines (but not the 7-azaindenoisoquinolines) by a decrease in the dispersion (electron correlation) contribution to π-π stacking.²³ In addition, the results of previous studies of indenoisoquinolines lacking substituents on the aromatic rings indicated that the 2,3-methoxy and 8,9-methylenedioxy substituents are not absolutely necessary for biological activity, but they do appear to consistently make a contribution to the anticancer activity.²⁶

### Chemistry

In an embodiment, processes for preparing compounds of formula (I) are described.

It was discovered that the known route to the analogous 2,3-methoxy and 8,9-methylenedioxy-indenoisoquinolines was unsuccessful in providing the targeted compounds. For example, the following compound failed to cyclize under known conditions.

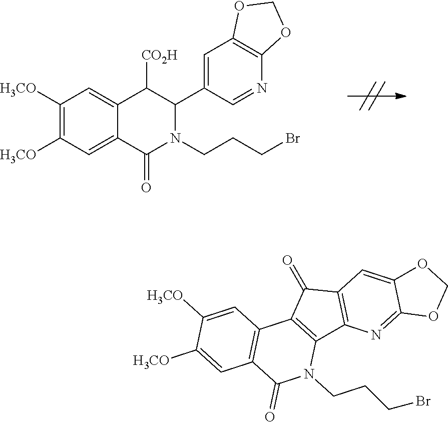

The route used to prepare 9-MeO-7-aza-indenoisoquinolines requires preparation of the following compound (WO 2018/118852 A1):

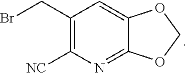 [SMILES: N#Cc1nc2c(cc1CBr)OCO2]

Routes to this intermediate based on known chemistry failed. The unexpected ring bromination shown below was a key step in preparing the required intermediate

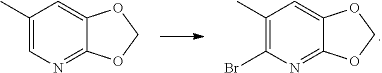

The processes described herein for preparing compounds of formula (I) overcome the obstacles posed by the previously known synthetic routes to 7-azaindenoisoquinolines. Illustrative processes include the following:

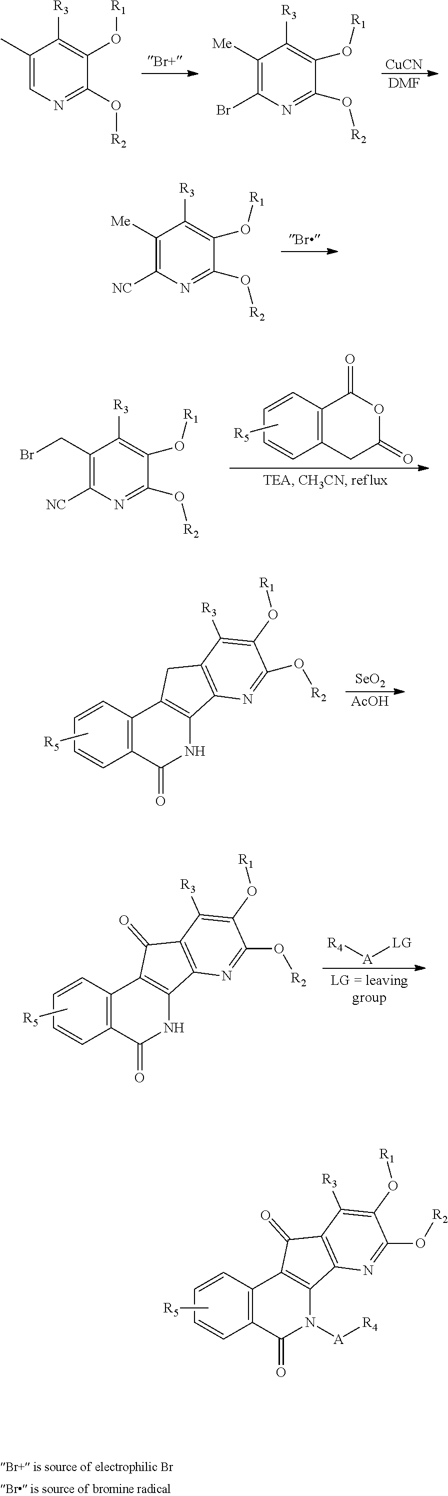

In another embodiment, compounds prepared by one or more of the processes described herein are provided.

Two different routes were used to synthesize the target compounds, depending on whether the 3-bromopropyl chain in the relevant intermediate was attached to the basic moiety in the side chain (16, Scheme 2) or to the 7-azaindenoisoquinoline (20, Scheme 3).

As outlined in Scheme 2, treatment of furaldehyde (5) with aqueous bromine under acidic conditions, followed by addition of sulfamic acid (6) to the reaction mixture, led to 5-bromopyridine-2,3-diol (7), which was converted to intermediate 8 in low (12%) yield with dibromomethane in the presence of potassium carbonate and cupric oxide.^{27, 28} Interestingly, the experimental details for the synthesis of 8 from 7 were never reported, although the 12% yield claimed for the conversion was duplicated as described herein.²⁸ The bromide of 8 was replaced by a methyl group using Suzuki-Miyaura cross coupling to afford the desired methylated substance 9.²⁹ It was surprisingly found that regioselective bromination of the 5-methylpyridine 9 on the aromatic ring with NBS in AcOH/CH₃CH at room temperature afforded the bromopyridine derivative 10,³⁰⁻³² which was followed by nucleophilic aromatic substitution with CuCN in DMF at reflux to yield intermediate 11. Free radical bromination of the methyl group of 11 with NBS in the presence of peroxybenzoic acid in refluxing carbon tetrachloride produced the desired primary bromide 12. Condensation of 12 with 6,7-dimethoxyhomophthalic anhydride in refluxing acetonitrile in the presence of triethylamine afforded 7-azaindenoisoquinoline 14,³³ which was oxidized with selenium dioxide to generate the penultimate intermediate 15.²³ Deprotonation of 15 with sodium hydride followed by alkylation of the resulting anion with the appropriate bromides 16 afforded the target compounds 17, 18, and 19.

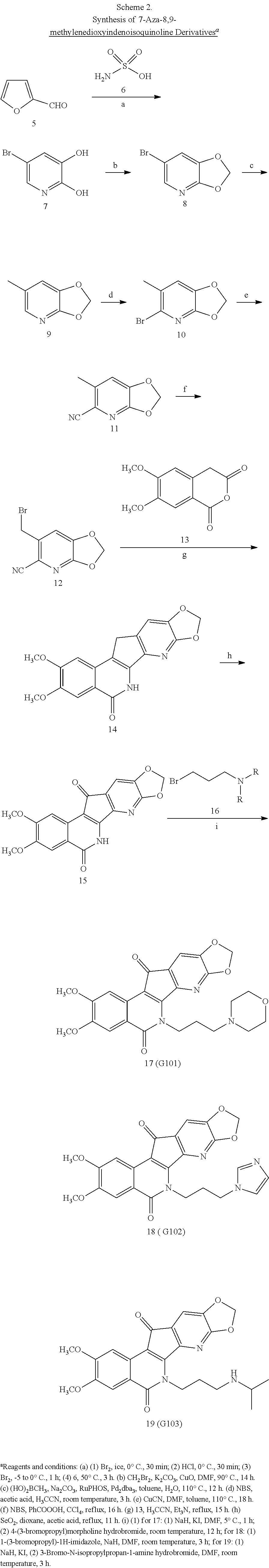

The first step in the alternate synthesis outlined in Scheme 3 involved deprotonation of intermediate 15 with sodium hydride followed by reaction with 1,3-dibromopropane in the presence of potassium iodide in DMF, resulting in the formation of the intermediate 20 having a 3-bromopropyl side chain.^{22,24} The target compounds shown in Scheme 3 were made by reacting 20 with the appropriate amines using DIPEA as the base in DMF for various times and temperatures as indicated in the legend.¹⁹

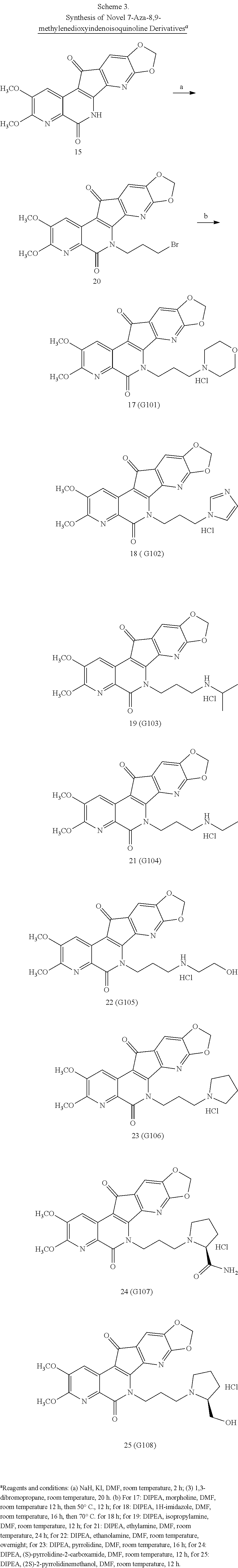

### Biological Results and Discussion.

1. In-Vitro Binding to the c-Myc Promoter G-Quadruplex (MycG4).

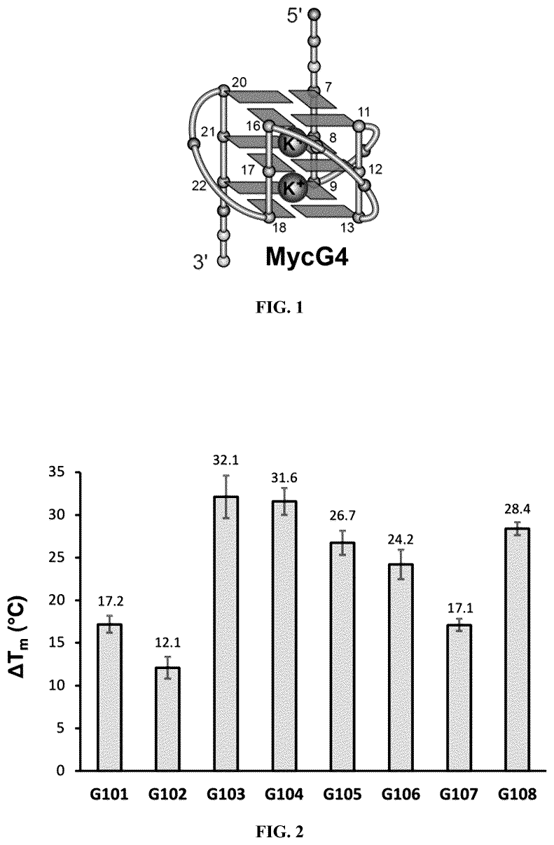

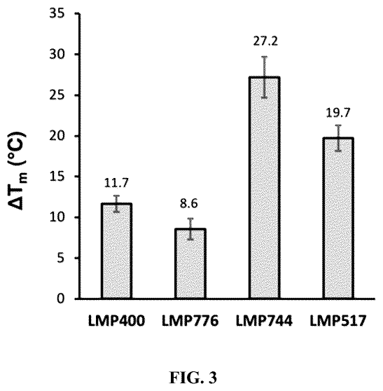

MycG4 Stabilization Measured by FRET Melting. A Förster Resonance Energy Transfer (FRET)-melting assay was conducted in order to determine whether the 7-aza-8,9-methylenedioxyindenoisoquinoline derivatives can bind and stabilize the c-Myc promoter G-quadruplex (MycG4, FIG. 1). MycG4 DNA was labeled with a FRET donor (6-FAM) at the 5′-end, and a FRET acceptor (TAMRA) at the 3′-end. Close proximity of the donor and acceptor fluorophores in the G-quadruplex secondary structure quenches the 6-FAM fluorescence due to FRET transfer to TAMRA. Melting of the secondary structure into single-strand DNA decreases the proximity of the FRET-pair, restoring 6-FAM-based fluorescence emission. Monitoring 6-FAM emission upon G-quadruplex thermal denaturation provides a melting curve from which a melting temperature (Tₘ), the temperature at which folded and unfolded DNA are equally populated, was derived. The Tₘ values of MycG4 were measured in the presence of the compounds in 10 mM K⁺ by FRET-melting experiments. All eight of the 7-aza-8,9-methylenedioxyindenoisoquinolines tested showed clear thermal stabilization (ΔTₘ) of the MycG4 (Table 1 and FIG. 2), with an increase of melting temperature greater than 10° C. Among them, G103, G104, G105, and G108 showed greatest thermal stabilization (ΔTₘ) of the MycG4. Data for the reference LMP compounds are provided in FIG. 3, and the structure of the reference compound 26 (LMP517) is displayed below.

TABLE 1
Increase in Melting Temperature, ΔTₘ, of MycG4 G-Quadruplex in the .
Presence of 7-Aza-8,9-methylenedioxyindenoisoquinolines
Determined by a FRET-Based Melting Experiment

| compound | ΔTₘ (° C.)^{a} | |
|---|---|---|
| G101 G102 G103 G104 G105 G106 G107 G108 | 17.2 ± 1.4 12.1 ± 1.7 32.1 ± 0.7 31.7 ± 0.8 26.7 ± 1.2 24.2 ± 1.6 17.1 ± 1.6 28.4 ± 0.26 | 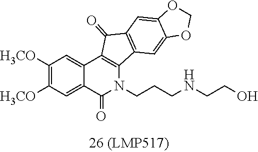 [SMILES: COc1cc2c3c(n(CCCNCCO)c(=O)c2cc1OC)-c1cc2c(cc1C3=O)OCO2] |

^{a}Conditions: 0.2 μM 5′-6-FAM/3′-TAMRA-MycG4 DNA, 2.0 μM (10 equiv) ligand, in 10 mM K⁺ buffer, pH 7.0.

Binding Affinity to the Myc G-Quadruplex. Binding affinity of eight 7-aza-8,9-methylenedioxyindenoisoquinolines to MycG4 was determined using a fluorescence binding assay with a 3′-TAMRA-labeled MycG4 DNA. Monitoring TAMRA emission upon drug titration afforded binding isotherms, from which apparent binding affinities, K_{d,app}, were derived. Six of the 7-aza-8,9-methylenedioxyindenoisoquinolines (G-103 to G-108) showed strong binding with apparent binding affinity K_{d,app} values smaller than 100 nM (Table 2). The indenoisoquinolines show negligible fluorescence in either the free or bound state. Data for the reference LMP compounds are provided in Table 3.

TABLE 2
Apparent Binding Affinity, K_{d}, of 7-Aza-
8,9-methylenedioxyindenoisoquinolines
to 3′-TAMRA-Labeled MycG4 DNA.

| compound | K_{d, app} (nM)^{a} |
|---|---|
| G101 | 249 |
| G102 | 147 |
| G103 | 77 |
| G104 | 55 |
| G105 | 97 |
| G106 | 55 |
| G107 | 59 |
| G108 | 14 |

^{a}Conditions: 20° C., 100 mM K⁺ buffer, pH 7.0.

TABLE 3
Reference Compound Results for the Data in
Table 3. Apparent Binding Affinity, K_{d}, of
LMP-Compounds to 3′-TAMRA-Labeled MycG4 DNA.

| compound | K_{d, app} (nM)^{a} |
|---|---|
| LMP400 | 301 |
| LMP744 | 11 |
| LMP776 | 69 |
| LMP517 | 109 |

^{a}Conditions: 20° C., 100 mM K⁺ buffer, pH 7.0.)

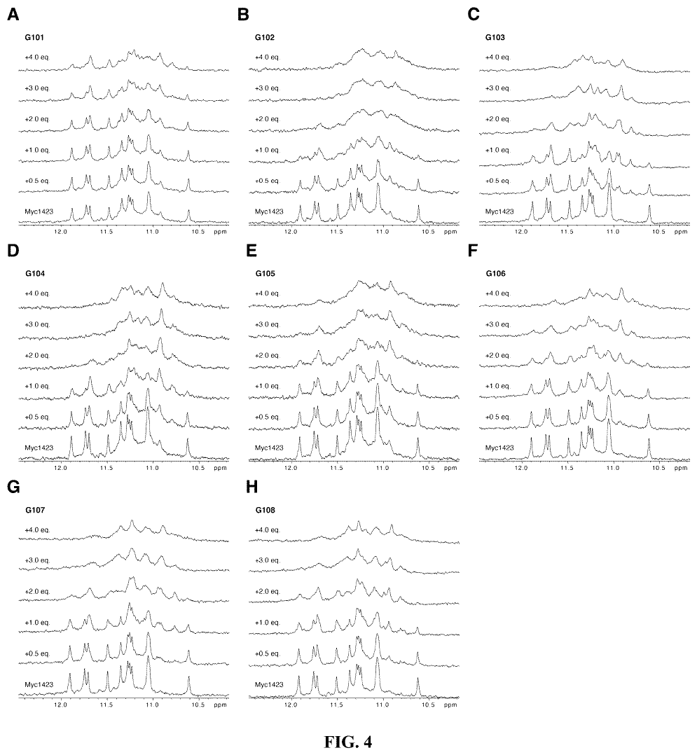

Binding Interactions with MycG4 by NMR. Binding interactions between 7-aza-8,9-methylenedioxyindenoisoquinolines and MycG4 were examined using 1D ¹H NMR titrations in K⁺-containing solution. The unbound Myc G-quadruplex shows twelve guanine imino protons, four from each of the three tetrads (FIG. 4). Changes in linewidth and chemical shift of imino proton resonances upon compound addition indicate binding to the G-quadruplex. Except for G101, all 7-aza compounds appear to bind MycG4 DNA at both ends, as shown by the clearly shifted imino proton peaks corresponding to the 3′- and 5′-tetrads upon drug addition. The binding appeared to be in the medium-to-fast exchange rate on the NMR timescale, as shown by the broadening of DNA proton peaks upon drug addition.

### 2. In Vivo Myc Inhibition

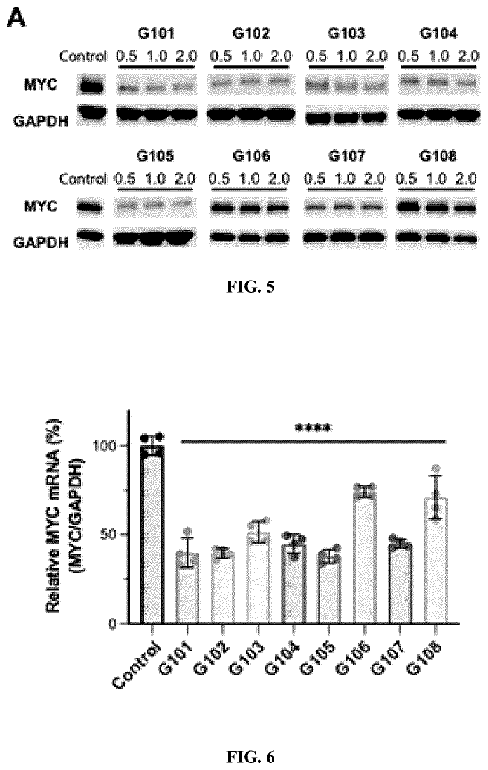

Western blot. G-quadruplex formed in the MYC promoter functions as a transcriptional silencer. To determine the effects of 7-aza-8,9-methylenedioxyindenosioquinolines on the MYC protein level, a western blotting experiment was carried out using MCF-7 breast cancer cells. Western blot experiments were conducted to examine the downregulation effects of 7-aza-8,9-methylenedioxyindenosioquinolines on Myc protein level (FIG. 5). MCF-7 breast cancer cells were incubated for 24 h at 0, 0.5, 1.0, and 2.0 μM of G-101-108. G101-G105 showed strong Myc inhibition effects. For G106-G108, only G107 appears to show moderate Myc inhibition while G106 and G108 show weak Myc downregulation.

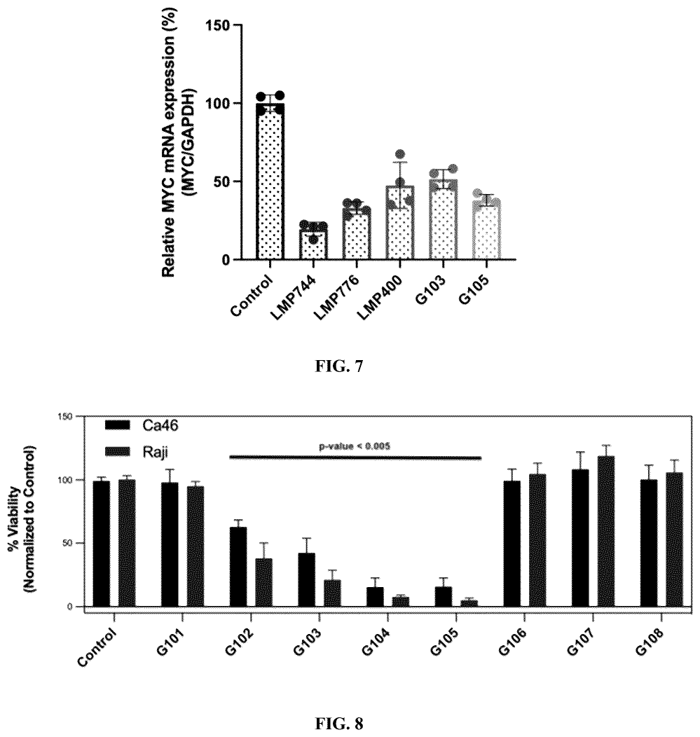

qRT-PCR. To confirm the effect on the transcription of the MYC gene in cancer cells by the selected 7-aza-8,9-methylenedioxyindenoisoquinolines, G103 and G105, the MYC mRNA levels in MCF-7 breast cancer cells were measured by qRT-PCR (FIG. 6). MCF-7 cells were incubated for 6 h at 0 and 1.0 μM of G103 and G105. Consistent with the western blotting data, G103 and G105 significantly lowered MYC mRNA levels at 6 hours post the treatments with 1.0 μM drugs. Data for the reference LMP compounds are provided in FIG. 7.

### 3. In Vivo Cytotoxicity Results in Cancer Cell Lines

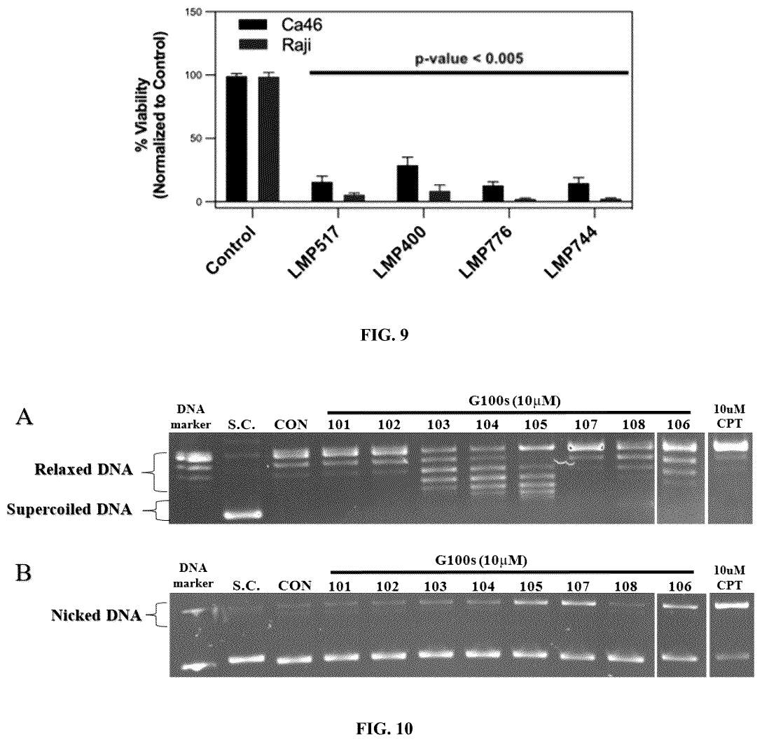

Single-Dosage Cell Viability Assay in CA-46 and Raji Cells. CA-46 and Raji are both Myc-dependent and -overexpressing non-Hodgkin's lymphoma cell lines. Both cell lines overexpress Myc due to a translocation that inserts an IgH enhancer upstream to the Myc gene. Whereas CA-46's translocation eliminates the G-quadruplex-forming region in one allele, Raji's translocation preserves the G-quadruplex-forming regions in both alleles. Therefore, Raji would be more sensitive to G-quadruplex-mediated Myc inhibition than CA-46, which may be reflected in lower IC₅₀ values for Raji than CA-46. To evaluate cell line selectivity, MTS single-dosage assays were conducted to compare the antiproliferative effects of 7-aza-8,9-methylenedioxyindenoisoquinolines on CA-46 and Raji cells at 250 nM drug concentration (FIG. 8). Average % viability values of CA-46 and Raji cells for each drug were presented in bar plots side-by-side. Among G101-G108 at 250 nM, G101 and G106-G108 did not show growth inhibition in either cell line. In contrast, G102-G105 showed clear anti-proliferative effects against both cell lines and statistically significant higher % viability of CA46 over Raji. Data for the reference LMP compounds are provided in FIG. 9.

Cytotoxicity Results in Raji Non-Hodgkin's Lymphoma Cells. MTS experiments were then conducted to test the anti-proliferative effects of 7-aza-8,9-methylenedioxyindenoisoquinolines in Raji cells. Cells were incubated at 2-2000 nM of each drug for 72 h, and cell viabilities were quantified using the MTS assay. The measured IC₅₀ values of all compounds are shown in Table 4. Among all compounds, G102-G105 are clearly more potent than G106-G108. This potency trend generally correlated with the western blot results, in which G106 and G108 not show weaker Myc downregulation. Data for the reference LMP compounds are provided in Table 5.

TABLE 4
7-Aza-8,9-methylenedioxyindenoisoquinoline
IC₅₀ (nM) Values in RAJI Lymphoma Cells.

| Drugs | RAJI |
|---|---|
| G101 | 340 |
| G102 | 153 |
| G103 | 167 |
| G104 | 131 |
| G105 | 147 |
| G106 | 1979 |
| G107 | 1708 |
| G108 | 1710 |

TABLE 5
Reference compound results for the data in Table
4. Non-7-aza-8,9-methylenedioxyindenoisoquinoline
IC₅₀ (nM) values in RAJI lymphoma cell line.

| Drugs | RAJI |
|---|---|
| LMP744 | 120 |
| LMP776 | 92 |
| LMP400 | 190 |
| LMP517 | 53 |

Cytotoxicity Results in Myc-dependent Cancer Cell Lines. Because of the high potencies of G101-G105 compounds displayed in Raji MTS IC₅₀ assay, these compounds were further tested in a selected panel of Myc-dependent/overexpressing cancer cell lines. Like in the CA-46 and Raji MTS IC₅₀ assay, each cell line was subject to 2-2000 nM of each compound for 72 h treatment.

Among 7-aza-8,9-methylenedioxyindenoisoquinolines (Table 6), G103-G105 were generally more potent than G101-G102 across all cell lines. Among all cell lines, MOLT-4, RS4;11, and CHLA-99, and RD-ES were very sensitive to G103-G105 with close to nM GI₅₀ values. Data for the reference LMP compounds are provided in Table 7.

TABLE 6
7-Aza-8,9-methylenedioxyindenoisoquinoline GI₅₀ (nM) Values in a
Selected Panel of Myc-Dependent/Overexpressing Cancer Cell Lines.

| Cell Lines | Cancer Types | G101 | G102 | G103 | G104 | G105 |
|---|---|---|---|---|---|---|
| CCRF-CRM | Leukemia | 248 | 117 | 57 | 49 | 57 |
| MOLT-4 | Leukemia | 113 | 43 | 19 | 13 | 12 |
| RS4; 11 | Leukemia | 283 | 120 | 53 | 38 | 34 |
| HL-60 | Leukemia | >2000 | >2000 | 328 | 270 | 290 |
| Kasumi-1 | Leukemia | >2000 | >2000 | 432 | 266 | 530 |
| THP-1 | Leukemia | 1319 | 1017 | 602 | 358 | 300 |
| K-562 | Leukemia | 1007 | 580 | 182 | 158 | 196 |
| ARD | Multiple Myeloma | 190 | 111 | 70 | 55 | 121 |
| AMO-1 | Multiple Myeloma | 1223 | 804 | 317 | 184 | 461 |
| L-363 | Multiple Myeloma | 342 | 232 | 126 | 96 | 154 |
| KMS-27 | Multiple Myeloma | 530 | 238 | 206 | 141 | 141 |
| RPMI-8226 | Multiple Myeloma | 905 | 481 | 234 | 190 | 559 |
| CHLA-32 | Ewing Sarcoma | 708 | 280 | 55 | 33 | 96 |
| CHLA-99 | Ewing Sarcoma | 526 | 265 | 89 | 58 | 31 |
| TC-106 | Ewing Sarcoma | 848 | 229 | 202 | 157 | 202 |
| RD-ES | Ewing Sarcoma | 608 | 180 | 31 | 20 | 22 |
| TC-71 | Ewing Sarcoma | 756 | 199 | 153 | 122 | 197 |
| TC-32 | Ewing Sarcoma | 569 | 127 | 55 | 53 | 104 |
| SK-N-MC | Ewing Sarcoma | 698 | 170 | 150 | 137 | 173 |
| MCF-7 | Breast Cancer | >2000 | >2000 | >2000 | >2000 | 1392 |
| MDA-MB- | Breast Cancer | >2000 | 1052 | 644 | 476 | 806 |
| 23 | | | | | | |

TABLE 7
Reference Compound Results for the Data in Table 6. LMPs and Camptothecin
GI₅₀ (nM) Values in a Selected Panel of Myc-Dependent/Overexpressing Cancer Cell Lines.

| Cell Lines | Cancer Types | LMP400 | LMP776 | LMP744 | LMP517 | CPT |
|---|---|---|---|---|---|---|
| CCRF-CRM | Leukemia | 316 | 70 | 57 | 11 | 4 |
| MOLT-4 | Leukemia | 239 | 42 | 4 | 3 | <2 |
| RS4; 11 | Leukemia | 336 | 59 | 30 | 7 | 6 |
| HL-60 | Leukemia | 1388 | 894 | 354 | 137 | 27 |
| Kasumi-1 | Leukemia | >2000 | 533 | 721 | 148 | 85 |
| THP-1 | Leukemia | 915 | 273 | 305 | 93 | 55 |
| K-562 | Leukemia | 864 | 341 | 219 | 74 | 22 |
| ARD | Multiple Myeloma | 126 | 33 | 77 | 27 | 11 |
| AMO-1 | Multiple Myeloma | 738 | 191 | 285 | 69 | 20 |
| L-363 | Multiple Myeloma | 214 | 53 | 119 | 42 | 10 |
| KMS-27 | Multiple Myeloma | 543 | 127 | 190 | 44 | 13 |
| RPMI-8226 | Multiple Myeloma | 989 | 139 | 620 | 82 | 14 |
| CHLA-32 | Ewing Sarcoma | 812 | 93 | 156 | 37 | 6 |
| CHLA-99 | Ewing Sarcoma | 773 | 276 | 77 | 44 | 4 |
| TC-106 | Ewing Sarcoma | 605 | 250 | 277 | 51 | 11 |
| RD-ES | Ewing Sarcoma | 472 | 227 | 24 | 9 | 5 |
| TC-71 | Ewing Sarcoma | 1057 | 232 | 244 | 58 | 8 |
| TC-32 | Ewing Sarcoma | 574 | 179 | 121 | 12 | 7 |
| SK-N-MC | Ewing Sarcoma | 1052 | 225 | 237 | 62 | 9 |
| MCF-7 | Breast Cancer | >2000 | >2000 | >2000 | 418 | 200 |
| MDA-MB-231 | Breast Cancer | 1903 | 329 | 968 | 319 | 37 |

High-level amplification of the MYC genes is observed in Ewing sarcoma, thus c-MYC is a target for Ewing sarcoma therapeutics. In Ewing sarcoma (EWS) cells, c-MYC is consistently highly expressed and the inducible expression of EWS-FLI-1 leads to strong upregulation of c-MYC. The G-quadruplex formed in the MYC promoter is a transvriptional silencer. LMP400 is a strong binder with the c-MYC promotor G-quadruplex and potently downregulates c-MYC. In contrast, camptothecin derivatives do not show appreciable binding to the c-MYC promoter G-quadruplex. Furthermore, preclinical and clinical studies show that G-quadruplex-stabilizing drugs, such as LMP400, show significant synergistic effects with PARP inhibitors and are very effective in tumors with HR-deficiencies such as Ewing sarcomas.

Based on the results of Table 7, above, the RD-ES cell line was used for in vivo studies to define the activity of LMP400 in animal xenograft model. Ten million RD-ES EWS cells were injected subcutaneously into 6-8 week old NOD-SCID mice. After randomization, once the tumor size was approximately 100 mm³, the animals were divided into 2 groups of 8 mice each. The control group was treated with vehicle and the other group was treated with i.v. 25 mg/kg LMP400. Body weights and tumor measurements were determined twice weekly. The results are shown in FIG. 10 as Mean±SEM of 8 animals.

Camptothecin derivatives do not sure appreciable binding to the c-MYC promoter G-quadruplex and they show no MYC-dependent anti-cancer activity in EWS. Furthermore, when combined with PARP inhibitors which are used to treat EWS, LMP400 has a significant synergy, increasing the effect of either drug alone.

### 5. Inhibition of Topoisomerase I

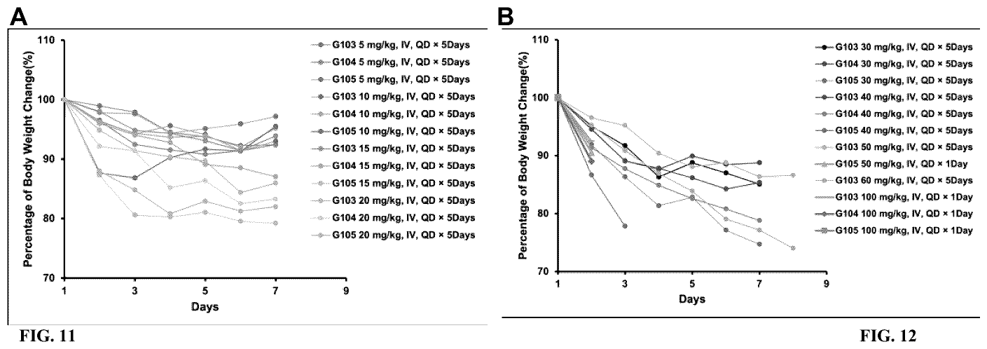

7-Aza-8,9-methylenedioxyindenoisoquinoline compounds were examined for inhibitory effects on Topol-mediated DNA relaxation (FIG. 11A), and for Topol poison effect using Topol-Mediated DNA Cleavage Assay (FIG. 11B).

Topol-Mediated DNA Relaxation Assay. Human Topoisomerase I Assay Kit (TG1015-1A) from Topogen was used to examine the inhibitory effects of compounds on Topo1-mediated DNA relaxation. Assay was performed according to manufacturer's instructions. A typical assay sample had a total volume of 20 μl including 100 ng of pHOTI DNA, 4 U of human Topol, and 10 μM test compound. Assay samples were incubated at 37° C. for 30 min followed by reaction termination with 10% SDS. This was followed by a 15 min proteinase K digestion at 37° C. to degrade Topo1 and a 1:1 volume chloroform:isoamyl alcohol DNA extraction. Samples were then electrophoresed on a 1% agarose gel in TBE buffer for 4 h at 46 v. Prior to imaging, gel was stained for 15 min with 0.5 μg/ml ethidium bromide solution and washed 3 times for 5 min intervals with Milli-Q water. The results are shown in FIG. 11A.

Topol-Mediated DNA Cleavage Assay. Human Topoisomerase I Assay Kit (TG1015-1A) from Topogen was used to examine the compounds for Topol poison effect. Assay was performed according to manufacturer's instructions. CPT at 10 μM was used as a positive control. A typical assay sample had a total volume of 20 μl including 100 ng of pHOTI DNA, 4 U of human Topol, and 10 μM test compound. Assay samples were incubated at 37° C. for 30 min followed by reaction termination with 10% SDS. This was followed by a 15 min Proteinase K digestion for 15 min at 37° C. to degrade Topo1 and a 1:1 volume chloroform:isoamyl alcohol DNA extraction. Samples were electrophoresed on a 1% agarose gel in TBE buffer for 2 h at 46 v. Both the agarose gel and buffer contained 0.5 μg/ml of ethidium bromide. The results are shown in FIG. 11B.

As shown by the data in FIG. 11A, none of the compounds inhibit the DNA cleavage reaction catalyzed by Top1. In contrast, compounds G103-G107 inhibit the DNA relegation reaction, while G101, G102 and G108 had minimal effects on DNA relegation as shown in FIG. 11B. In other words, G103-G107 act as Top1 poisons and not Top1 suppressors, with G105-G1-7 being the most potent. This is consistent with the Top1 inhibitory activities of the indenoisoquinolines having a 7-carbon atom.

### 6. Pharmacokinetics Results

Pharmacokinetics. The pharmacokinetics results obtained after IV and PO administration of compounds 19, 21, and 22 to CD1 mice are listed in Tables 8 and 9, respectively. The half-lives of the drugs after IV administration were 3.29 h for 19, 2.73 h for 21, and 1.84 h for 22. After oral administration, the half-lives were 6.88 h for 19, 6.91 h for 21, and 5.20 h for 22. There was significant variability in the fraction of the dose absorbed after oral administration, with 41% of the dose of 19 absorbed, 14.2% of the dose of 21 absorbed, and 1.19% of the dose of 22 absorbed. The absorption percentages are in general agreement with the C Log P values of 1.67 for 19, 1.36 for 21, 0.363 for 22, indicating the expected higher absorption of more lipophilic compounds from the GI tract.

TABLE 8
IV Pharmacokinetic Parameters (Dose = 1 mg/kg) for Compounds
19 (G103), 21 (G104), and 22 (G105) in Male CD1 Mice.

| PK parameters | Units | 19 | 21 | 22 |
|---|---|---|---|---|
| Cl_obs | mL/min/kg | 99.2 | 107 | 107 |
| T_{1/2} | h | 3.29 | 2.73 | 1.84 |
| C₀ | ng/mL | 67.4 | 119 | 177 |
| AUCₗₐₛₜ | h · ng/mL | 143 | 142 | 151 |
| AUC_{Inf} | h · ng/mL | 168 | 156 | 155 |
| AUC_{—}_{% Extrap}_{_{—}}obs | % | 14.8 | 8.98 | 2.61 |
| MRT_{Inf}_{_{—}}obs | h | 3.72 | 2.66 | 1.49 |
| AUCₗₐₛₜ/D | h · ng/mL | 143 | 142 | 151 |
| Vₛₛ_{_{—}}obs | L/kg | 22.2 | 17 | 9.62 |

TABLE 9
PO Pharmacokinetic Parameters (Dose = 10 mg/kg) for
Compounds 19 (G103), 21 (G104), 22 (G105) in Male CD1 Mice.

| PK parameters | Unit | 19 | 21 | 22 |
|---|---|---|---|---|
| T_{1/2} | h | 6.88 | 6.91 | 5.20 |
| Tₘₐₓ | h | 2.00 | 0.25 | 0.500 |
| Cₘₐₓ | ng/mL | 47.1 | 16.2 | 3.50 |
| AUCₗₐₛₜ | h · ng/mL | 643 | 202 | 18.0 |
| AUC_{Inf} | h · ng/mL | 704 | 222 | 28.8 |
| AUC_{—}_{% Extrap}_{_{—}}obs | % | 8.61 | 8.81 | 37.3 |
| MRT_{Inf}_{_{—}}obs | h | 9.64 | 9.43 | 7.86 |
| AUCₗₐₛₜ/D | h · ng/mL | 64.3 | 20.2 | 1.80 |
| F | % | 41.9 | 14.2 | 1.19 |

Plasma Protein Binding and Stability Analysis. The protein binding percentages and stabilities of compounds 19, 21, and 22 were determined in both human and mouse blood plasma. The results are listed in Table 10. The rank order of protein binding followed the lipophilicities of the compounds, with compound 19 being the most highly protein bound and compound 22 being the least highly protein bound in both mouse and human plasma. The free drug (unbound) concentrations of all three compounds are sufficient to drive pharmacological efficacy, and all three compounds are also relatively stable in blood plasma.

TABLE 10
Protein Binding Results of Compounds 19, 21, 22,
and Control Compound in Human and Mouse Plasma

| | | | | % |
|---|---|---|---|---|
| Compound | | | | Stability |
| ID | Species | % Bound | % Recovery | at 6 hrs |
| Ketoconazole | | 98.01 | 107.45 | 104.96 |
| 19 (G103) | Human | 82.60 | 88.05 | 94.96 |
| 21 (G104) | | 81.21 | 83.58 | 86.15 |
| 22 (G105) | | 80.88 | 99.38 | 95.42 |
| Ketoconazole | | 99.26 | 93.23 | 88.82 |
| 19 (G103) | Mouse | 84.11 | 88.02 | 92.63 |
| 21 (G104) | | 83.44 | 80.60 | 86.57 |
| 22 (G105) | | 79.41 | 90.43 | 97.59 |

Stability Analysis in Human and Mouse Liver Microsomes. The stabilities of compounds 19, 21, and 22 were determined in the presence of human and mouse liver microsomes and the results are listed in Table 11. All three compounds displayed robust microsomal stabilities consistent with their in vivo stabilities in male CD1 mice (Table 11). The half-lives vs. human microsomes were in the 227-372 min range, with the order of stabilities being 19>21>22. Interestingly, compounds 19 and 21 were both more stable vs. human microsomes than mouse microsomes, but for compound 22, the order was reversed, with greater stability vs. the mouse microsomes than the human microsomes. In spite of the larger half-life and smaller in vitro CLᵢₙₜ observed for 22 in mouse vs. human microsomes, the calculated scaled-up CLᵢₙₜ was greater for the mouse due to the much larger scaling factor for mouse vs. human (Table 20a, Experimental Section).

TABLE 11
Metabolic Stability of Test Compounds in Pooled
Human and Male Mouse Liver Microsomes

| Compound | | | in vitro CLᵢₙₜ | Scaled-up CLᵢₙₜ |
|---|---|---|---|---|
| ID | Species | T_{1/2} (min) | (μL/min/mg protein) | (mL/min/Kg) |
| Verapamil | Human | 9.40 | 147.50 | 184.99 |
| | Mouse | 3.14 | 441.26 | 1930.52 |
| 19 (G103) | Human | 372.37 | 3.72 | 4.67 |
| | Mouse | 220.77 | 6.28 | 27.47 |
| 21 (G104) | Human | 263.87 | 5.25 | 6.59 |
| | Mouse | 157.99 | 8.77 | 38.38 |
| 22 (G105) | Human | 226.52 | 6.12 | 7.67 |
| | Mouse | 429.95 | 3.22 | 14.10 |

Caco-2 Cellular Permeability Studies. Caco-2 permeability studies on compounds 19, 21, and 22 were performed and the results are listed in Table 12. All three compounds showed high efflux ratios in Caco-2 cells, indicating that all three compounds should be substrates of efflux transporter(s) (P-gp, BCRP and/or MRP2). All three compounds showed moderate permeability in the apical to basolateral direction, which is the direction for intestinal absorption.

TABLE 12
Permeability Results of Test Compounds in Caco-2 Cells

| | P_{app (A→B)} | P_{app (B→A)} | | | |
|---|---|---|---|---|---|
| Compound | (10⁻⁶, | (10⁻⁶, | Efflux | Recovery | Recovery |
| ID | cm/s) | cm/s) | Ratio | (%)AP-BL | (%)BL-AP |
| Propranolol | 25.34 | 17.63 | 0.70 | 80.74 | 92.73 |
| Digoxin | 0.74 | 18.71 | 25.25 | 88.64 | 106.70 |
| Prazosin | 9.28 | 31.47 | 3.39 | 79.64 | 94.99 |
| 19 (G103) | 1.55 | 8.95 | 5.77 | 36.17 | 75.54 |
| 21 (G104) | 1.69 | 16.04 | 9.51 | 54.60 | 81.64 |
| 22 (G105) | 2.39 | 18.16 | 7.60 | 75.07 | 95.77 |

To summarize, compound 19 (G103) has the longest IV half-life, greatest % absorption PO, greatest stability vs. human liver microsomes, and lowest efflux ratio in Caco-2 cells. Compound 21 (G104) has the best cytotoxicity vs. CA46 and Raji cells, and excellent selectivity for Raji cells that is comparable to 22 (G105).

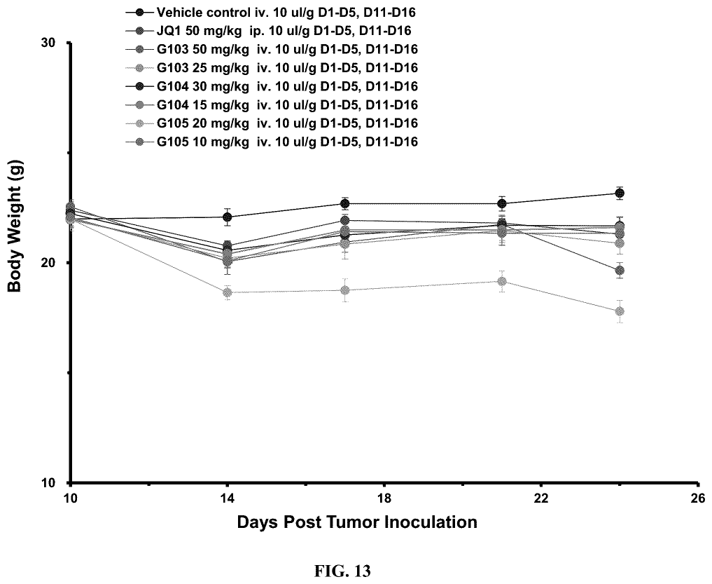

Maximum Tolerated Dose Determinations. The maximum tolerated doses (MTDs) of G103, G104, and G105 were determined in NOD SCID mice and the results are displayed in Table 13 and in FIGS. 12 and 13. The MTD of G103 was 50 mg/kg, while those of G104 and G105 were 30 mg/kg and 20 mg/kg, respectively. Experiments involving 100 mg/kg doses of all three drugs were terminated after one day because of toxicity, while the study involving 50 mg/kg dose of G105 was also halted after one day (FIG. 12). Similarly, the investigation with a 40 mg/kg dose of G105 was discontinued after two days (FIG. 13). The large difference in MTDs between G103 and G104 (50 vs. 30 mg/kg) is unexpected because the change in chemical structure is small (one methyl group vs. a hydrogen in the side chain).

TABLE 13
Determination of Maximum Tolerated Doses (MTDs)

| Compd | Dose (mg/Kg)^{a} | Body Weight %^{b} |
|---|---|---|
| G103 | 5 | 92.4 |
| | 10 | 93 |
| | 15 | 93.9 |
| | 20 | 86 |
| | 30 | 85.1 |
| | 40 | 85.3 |
| | 50^{c} | 86.6 |
| | 60 | 74 |
| G104 | 5 | 92.4 |
| | 10 | 95.2 |
| | 15 | 87 |
| | 20 | 83.3 |
| | 30^{c} | 88.8 |
| | 40 | 78.8 |
| G105 | 5 | 97.2 |
| | 10 | 95.5 |
| | 15 | 79.2 |
| | 20^{c} | 82 |
| | 30 | 74.7 |

^{a}IV, QD x 5.
^{b}The average percentage of the initial body weight observed 7 days after the first dose based on four mice per group.
^{c}Maximum tolerated dose, with a tolerated dose defined by a body weight % of ≥80%.

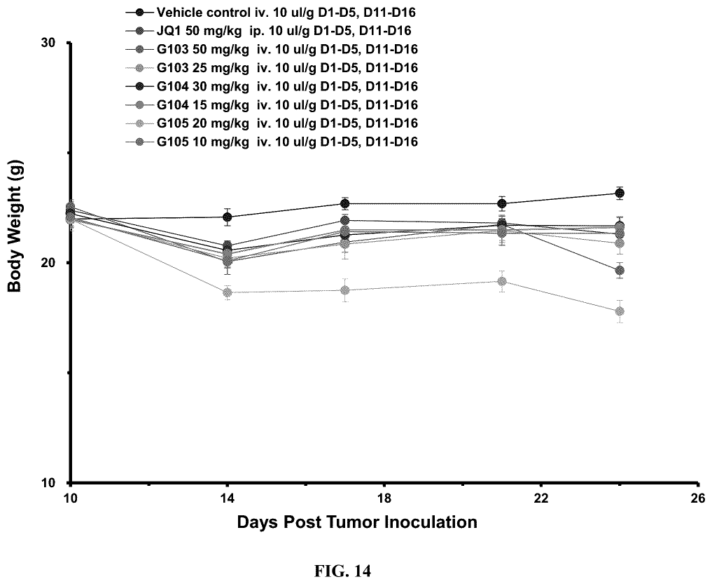

Xenograft Studies. An investigation of the efficacies of G103, G014, and G105 was initiated in female NOD SCID mice after inoculation with MOLT-4 human leukemia cells, RD-ES human sarcoma cells, and RS4; 11 human leukemia cells. JQ1 was employed as a positive control. Treatment was begun 10 days after inoculation, when the tumor had reached a size of about 100-150 mm³. The drugs were administered by IV on days 1-5 and 11-16 after treatment began, and body weights and tumor values were monitored on days 10, 14, 17, 21, and 24 after inoculation. As shown in FIG. 14, the drugs were in general relatively well tolerate as judged by decreases in body weight, with the maximum decreased observed after administration of G105 at does 20 mg/kg (average animal weight 80.9±1.1% relative to initial body weight 24 days after tumor inoculation).

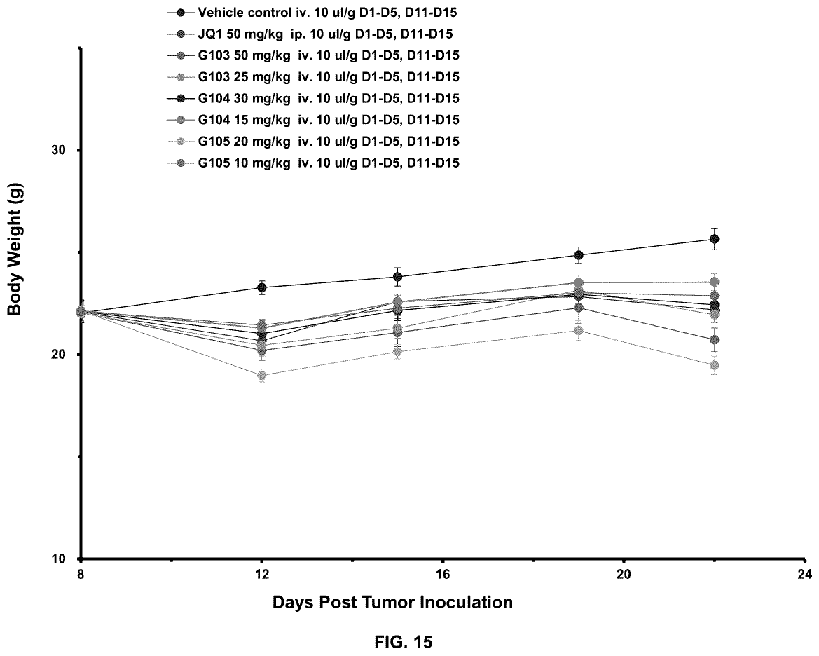

FIG. 15 displays plots of tumor size vs. time after inoculation of female NOD SCID mice with MOLT-4 human leukemia cells and IV treatment with various doses of G103, G104, and G105, as well as IP administration of the positive control JQ1. Each of the “G compounds” was administered in higher and lower doses as detailed in FIG. 14. The data reveal the relative efficacies of the various treatments: G105 (20 mg/kg)>G103 (50 mg/kg)>G103 (25 mk/kg)>G105 (10 mg/kg)>G104 (30 mg/kg)>G104 (15 mg/kg)>JQ1 (50 mg/kg). Each of the drugs decreased the rate of increase of tumor size relative to control.

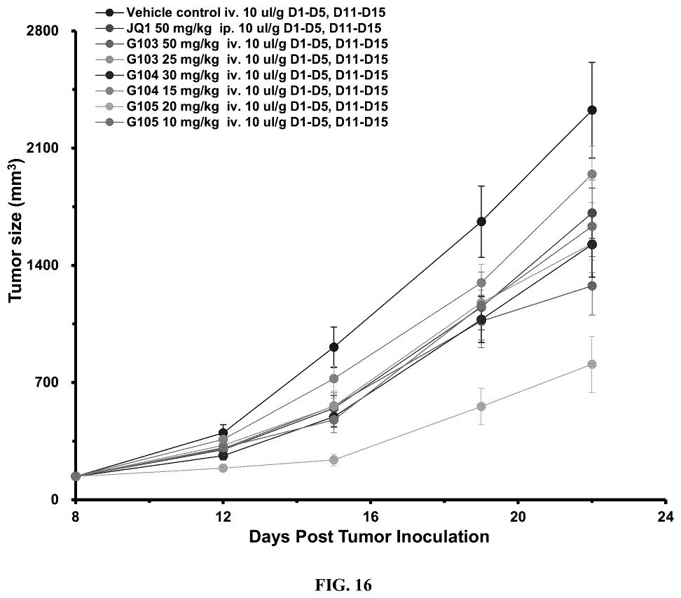

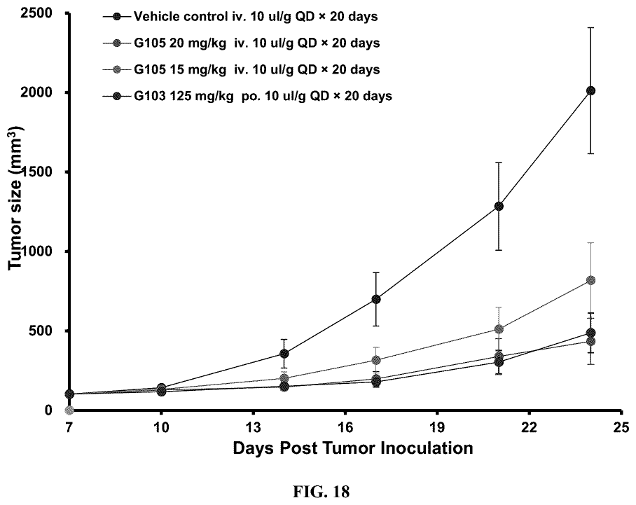

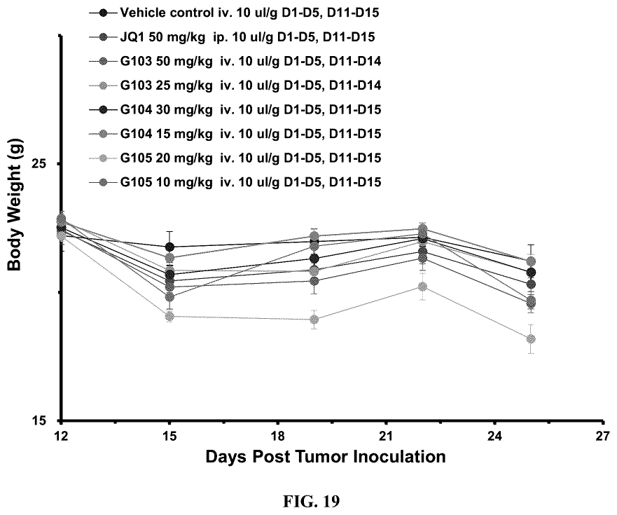

Similar studies were performed after inoculation of female NOD SCID mice with RD-ES human sarcoma tumor cells (FIGS. 16-19). The tumors grew much more rapidly and treatment was started on day 8 after tumor inoculation, with measurements of body weights and tumor sizes being made on days 8, 12, 15, 19, and 22 after tumor inoculation. The most potent treatment was the same as with MOLT-4, with G105 at a dose of 20 mg being the most potent. However, the overall order of potencies was not the same as above: G105 (20 mg/kg)>G103 (50 mg/kg)>G104 (30 mg/kg)>G103 (25 mg/kg)>G105 (10 mg/kg)>JQ-1 (50 mg/kg)>G104 (15 mg/kg). The average RD-ES tumor sizes were much larger than observed with MOLT-4 (2327±285 vs. 546±59 mm³).

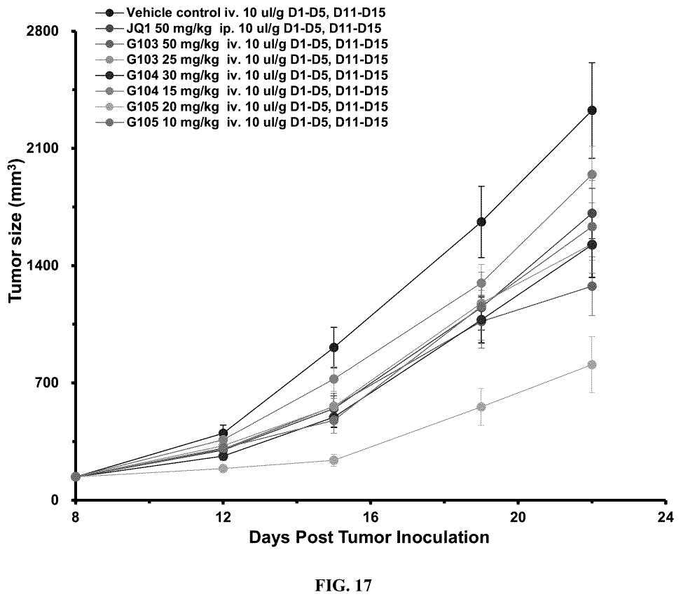

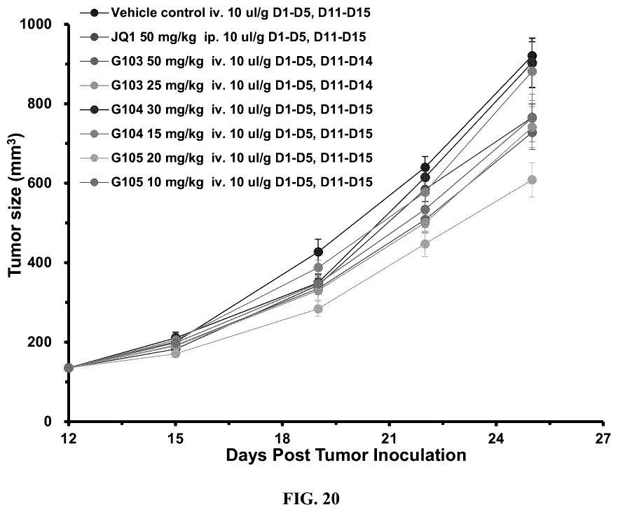

The compounds were also evaluated in an RS4;11 human leukemia xenograft model (FIGS. 20 and 21). Treatment was started 12 days after tumor inoculation, with drugs being administered on days 1-5 and 11-15 after the start of treatment. Animal weights and tumor volumes were determined at 12, 15, 19, 22, and 25 days after tumor inoculation. As in the other cases, treatment with G105 at the 20 mg/kg dose level turned out to be the most toxic as estimated from the decrease in animal weight (FIG. 17), and it was also the most potent in reduction tumor volume (FIG. 20). The relative potencies of the various treatments vs. RS4; 11 were distinct from those observed in the other two cases: G105 (20 mg/kg)>G103 (50 mg/kg)>G103 (25 mg/kg)>G105 (10 mg/kg)>JQ-1 (50 mg/kg)>G104 (15 mg/kg)>G104 (30 mg/kg). Overall, the various treatments had low to moderate efficacies in the RS4;11 xenograft model, with tumor volumes after G104 treatment at both dose levels (15 and 30 mg/kg) being close to that observed in untreated animals at the 25 day time point.

Brain Penetration Studies. One possible application of the indenoisoquinolines is in the treatment of malignant brain tumors. Prior studies have demonstrated CNS penetration by LMP400 (2), suggesting that G103, G104, and G105 might also be able to penetrate the blood-brain barrier.⁴⁰ The concentrations of these compounds, as well as LMP744 (4), in the blood plasma and brain were therefore monitored by LC-MS/MS after IV administration to CD1 mice and the brain/plasma concentrations were calculated (Table 14a). The four compounds were tested by IV administration of a dose of 20 mg/kg to four groups of twenty-four male CD1 mice with three animals used for each time point, for a total of ninety-six animals utilized to generate the data in Table 14a, and the blood plasma and brain tissue samples were collected from each mouse. The concentrations were determined by LC-MS/MS with direct measurement of the plasma samples. The brain tissue was homogenized with water using a 3:1 ratio of water (mL) to brain (g) before determination of the concentrations (ng/mL) in the brain homogenates, and the homogenate values then were multiplied by a factor of 4 to obtain the brain concentrations (ng/g). The ng/g concentration values in brain were then converted to nM concentrations in brain using the conversion factor 1.03 based on the fact that 1 cc of brain weighs 1.03 g.⁴¹ The initial brain concentrations were proportional to the lipophilicities of the side chains, while the initial plasma concentrations were inversely proportional to the lipophilicities of the side chains, reflecting the more rapid initial partitioning of the compounds with more lipophilic side chains fro plasma into brain. The highest initial brain concentrations were achieved with G103, with a mean brain/plasma ratio of 7.97 observed 15 min after administration, and relatively high brain/plasma ratios of G103 were maintained throughout the 24 h observation period. The second-highest initial brain concentrations and mean brain/plasma ratios were detected with G104, which produced a ratio of 1.88 at the 15 min time point. Lower initial brain concentrations and ratios were observed with G105 and LMP744. Although low concentrations of the compounds were measured in both plasma and brain at the 24 h time point, high brain/plasma ratios were calculated, with higher numbers observed for the compounds with the lowest initial ratios. This reflects the fact that the brain concentrations of G105 and LMP744 were relatively low but surprisingly stable throughout the 24-hour time period, while G103 and G104 were associated with “normal” brain pharmacokinetics, with the concentrations of G103 and G104 decreasing as expected over time. On the other hand, the plasma concentrations of all of the compounds decreased as expected as a function of time.

A similar analysis was performed with LMP744 and LMP400 in combination with Temodar and Lynparza. In Table 14b, the brain concentrations, plasma concentrations, and brain/plasma ratios of LMP744 and LMP400 are listed when administered in combination with 100 mg/kg PO of Lynparza or 125 mg/kg of Temodar. Clearly, LMP400 is able to penetrate the blood-brain barrier more effectively than LMP744, as evidenced by the much higher brain concentrations and brain/plasma ratios of LMP400 vs. LMP744. The lower penetration of the compound with the hydroxyethylaminopropyl side chain (i.e., LMP744) documented in Table 14b is consistent with the results provided in Table 14a, which document that lower levels of LMP744 and G105 relative to G103 and G104. It is interesting to note that Lynparza seems to facilitate the penetration of LMP400 into the brain relative to Temodar, as the brain concentrations of LMP are consistently higher after administration with Lynparza at all of the time points. Finally, the “flat” concentration levels of LMP744 as a function of time that are shown in Table 14b are also consistent with the results with both LMP744 and G105 displayed in Table 14a, indicating that the hydroxyethylaminopropyl side chain seems to hold relatively low levels of these drugs in the brain for long periods of tie (5 min to 24 h after administration). This is in contrast to LMP400, which shows “normal” decrease on concentrations as a function of time.

The concentrations of Temodar and Lynparza during these experiments were also traced as shown in Table 14c. Temodar is able to get into the brain after oral administration, while the partitioning of Lynparza into the brain after oral administration is very inefficient. Based on these results, the IV use of LMP400 in combination with orally administered Temodar might be effective, but the combination with Lynparza does not appear to be promising for the treatment of CNS cancers.

The pharmacokinetics observed during plasma and brain monitoring are detailed in Tables 15a and 16a. G103 and G104 appeared rapidly in brain tissue after IV administration (Tₘₐₓ 5 min for both compounds, Table 16a), and the plasma half-life for G104 was 7.06 h (Table 14a). A valid plasma half-life for G103 could not be calculated because the AUC__{%Extrap_}obs was over 20%. The plasma half-lives of G104 and G105 observed after the 20 mg/kg dose (Table 15a) are longer than those seen after the 1 mg/kg dose (Table 8), although they are in the same order (G104 T_{1/2}>G105 T_{1/2}). It was not possible to calculate valid brain T_{1/2}, AUC_{Inf} and MRT_{Inf}_obs parameters because the AUC__{%Extrap_}obs values for G103, G104, G105, and LMP744 in the brain were all well over 20% (Table 16b). The initial brain concentrations of G103 are significantly higher than G104 (Cₘₐₓ 3809 vs. 1613 ng/g, respectively, Table 16a), which reflect the greater lipophilicity of G103 vs. G104 (c Log P values=1.674 vs. 1.365, respectively).

The plasma and brain pharmacokinetic parameters for LMP400 and LMP744 when administered in combination with Lynparza and Temador are displayed in Table 15b and 16b, respectively. LMP400 appeared rapidly in the brain, as indicated by the Tₘₐₓ of 5 min (0.0830 h, Table 16b). On the other hand, the Tₘₐₓ for LMP744 was 1 h in the presence of Lynparza and 2 h in the presence of Temador (Table 16b), both being delayed relative to the 0.25 h observed when LMP744 was administered alone (Table 16a). As expected, the brain Cₘₐₓ values of LMP400 are significantly higher than LMP744 regardless of co-administration of Temador or Lynparza, and both Temador and Lynparza do not affect the Cₘₐₓ of LMP744 (compare values in Table 16b with 16a).

It should be noted that the plasma C₀ AUCₗₐₛₜ, and AUC_{Inf} values for LMP 744 are different when co-administered with Lynparza vs. Temador (Table 15b). LMP744 has a much greater volume of distribution at steady state then LMP400. The half-lives and volumes of distribution at steady state are not significantly affected by being administered with Lynparza vs. Temador.

TABLE 14a
Mouse Brain and Plasma Concentrations and Brain/Plasma Ratios after IV Administration of 20 mg/kg of G103, G104, G105, and LMP744

| | G103 | | | G104 | | | G105 | | | LMP744 | | |
|---|---|---|---|---|---|---|---|---|---|---|---|---|
| | | | Brain/ | | | Brain/ | | | Brain/ | | | Brain/ |
| | Brain | Plasma | Plasma | Brain | Plasma | Plasma | Brain | Plasma | Plasma | Brain | Plasma | Plasma |
| Time | Conc | Conc | Conc | Conc | Conc | Conc | Conc | Conc | Conc | Conc | Conc | Conc |
| (h) | (nM) | (nM) | Ratio | (nM) | (nM) | Ratio | (nM) | (nM) | Ratio | (nM) | (nM) | Ratio |
| 0.063 | 8690 ± 1563 | 1146 ± 183 | 7.58 | 3865 ± 584 | 2385 ± 501 | 1.55 | 973 ± 189 | 5579 ± 594 | 0.17 | 826 ± 57 | 2984 ± 218 | 0.27 |
| 0.25 | 8298 ± 1798 | 3040 ± 120 | 7.97 | 3585 ± 566 | 1911 ± 308 | 1.88 | 589 ± 172 | 2330 ± 58 | 0.42 | 941 ± 158 | 1434 ± 123 | 0.65 |
| 0.5 | 5076 ± 321 | 543 ± 58 | 7.90 | 3036 ± 563 | 1512 ± 1057 | 2.40 | 963 ± 257 | 2184 ± 561 | 0.44 | 582 ± 380 | 1712 ± 945 | 0.34 |
| 1 | 3956 ± 170 | 564 ± 70 | 6.55 | 3029 ± 139 | 796 ± 151 | 3.81 | 838 ± 80 | 1145 ± 306 | 0.73 | 802 ± 122 | 894 ± 194 | 0.90 |
| 2 | 1783 ± 254 | 548 ± 20 | 3.21 | 2023 ± 581 | 358 ± 75 | 5.65 | 983 ± 191 | 678 ± 540 | 1.45 | 743 ± 136 | 535 ± 103 | 1.39 |
| 4 | 979 ± 26 | 357 ± 39 | 2.74 | 969 ± 348 | 271 ± 45 | 3.58 | 836 ± 152 | 309 ± 177 | 2.71 | 857 ± 162 | 154 ± 14 | 5.44 |
| 8 | 867 ± 31 | 267 ± 23 | 3.25 | 896 ± 185 | 144 ± 19 | 6.22 | 944 ± 47 | 52 ± 8 | 10.2 | 759 ± 188 | 86 ± 31 | 6.85 [symbol] |
| 24 | 583 ± 69 | 105 ± 5 | 5.70 | 575 ± 266 | 35 ± 9 | 16.4 | 913 ± 100 | 25 ± 2 | [symbol] 36.5 | 524 ± 166 | 23 ± 5 | 27.1 |

[symbol]

Three animals were used in the determination of the data per time point for each compound, for a total of 96 animals used to generate the data the table. [symbol]
indicates data missing or illegible when filed

TABLE 14b
Mouse Brain and Plasma Concentrations and Brain/Plasma Ratios of LMP744 and LMP400 after IV Administration of
10 or 20 mg/kg of LMP744 or LMP400 in combination with 100 mg/kg PO of Lynparza of 125 mg/kg PO of Temador

| | LMP744 | | | LMP744 | | | LMP400 | | |
|---|---|---|---|---|---|---|---|---|---|
| | (10 mg/kg) + Lynparza | | | (20 mg/kg) + Temador | | | (10 mg/kg) + Lynparza | | |
| | | | Brain/ | | | Brain/ | | | Brain/ |
| | Brain | Plasma | Plasma | Brain | Plasma | Plasma | Brain | Plasma | Plasma |
| Time | Conc | Conc | Conc | Conc | Conc | Conc | Conc | Conc | Conc |
| (h) | (nM) | (nM) | Ratio | (nM) | (nM) | Ratio | (nM) | (nM) | Ratio |
| 0.063 | 694 ± 123 | 3126 ± 282 | 0.22 | 1238 ± 186 | 6605 ± 848 | 0.19 | 26089 ± 1574 | 4291 ± 229 | 6.08 |
| 0.25 | 89 ± 19 | 1356 ± 229 | 0.46 | 1093 ± 168 | 2238 ± 465 | 0.49 | 14924 ± 107 | 3258 ± 295 | 4.58 |
| 0.5 | 780 ± 24 | 940 ± 155 | 0.83 | 1199 ± 243 | 1854 ± 101 | 0.89 | 12083 ± 1787 | 2231 ± 419 | 5.42 |
| 1 | 825 ± 250 | 402 ± 29 | 2.05 | 1390 ± 145 | 1027 ± 202 | 1.35 | 6670 ± 431 | 1787 ± 169 | 5.73 |
| 2 | 850 ± 58 | 322 ± 88 | 2.64 | 1328 ± 188 | 534 ± 143 | 2.49 | 3766 ± 408 | 1381 ± 84 | 2.73 |
| 4 | 548 ± 28 | 188 ± 54 | 2.91 | 1272 ± 188 | 355 ± 37 | 3.58 | 2666 ± 265 | 941 ± 190 | 2.83 |
| 8 | 557 ± 66 | 72 ± 26 | 7.74 | 1244 ± 78 | 139 ± 17 | [symbol] 6.95 | 1162 ± 104 | 491 ± 53 | 2.57 |
| 24 | 457 ± 61 | 13 ± 2 | 20.30 | 955 ± 76 | 21 ± 4 | 46 | 235 ± 76 | 111 ± 39 | 2.32 |
| | | | | | | | LMP400 | | |
| | | | | | | | (10 mg/kg) + Temador | | |
| | | | | | | | | | Brain/ |
| | | | | | | | Brain | Plasma | Plasma |
| | | | | | | Time | Conc | Conc | Conc |
| | | | | | | (h) | (nM) | (nM) | Ratio |
| | | | | | | 0.063 | 19545 ± 2371 | 4764 ± 524 | 4.10 |
| | | | | | | 0.25 | 13838 ± 638 | 2311 ± 274 | 5.99 |
| | | | | | | 0.5 | 10074 ± 1157 | 1935 ± 274 | 5.20 |
| | | | | | | 1 | 5358 ± 276 | 1944 ± 211 | 2.75 |
| | | | | | | 2 | 5289 ± 321 | 1041 ± 57 | 3.16 |
| | | | | | | 4 | 1722 ± 226 | 617 ± 24 | 2.79 |
| | | | | | | 8 | 919 ± 165 | 481 ± 104 | 1.91 |
| | | | | | | 24 | 177 ± 70 | 183 ± 30 [symbol] | 0.96 |

[symbol]

Three animals were used in the determination of the data per time point for each compound, for a total of 96 animals used to generate the data the table. [symbol]
indicates data missing or illegible when filed

TABLE 14c
Mouse Brain and Plasma Concentrations and Brain/Plasma Ratios of Temador or Lynparza after PO Administration of 125 mg/kg of
Temador or 100 mg/kg of Lynparza and IV Administration of 20 mg/kg of LMP744 or 10 mg/kg of LMP744 or 10 mg/kg LMP400.

| | Temadar + LMP744 (20 mg/kg) | | | Temadar + LMP400 (10 mg/kg) | | | Lynparza + LMP744 (10 mg/kg) | | |
|---|---|---|---|---|---|---|---|---|---|
| | | | Brain/ | | | Brain/ | | | Brain/ |
| | Brain | Plasma | Plasma | Brain | Plasma | Plasma | Brain | Plasma | Plasma |
| Time | Conc | Conc | Conc | Conc | Conc | Conc | Conc | Conc | Conc |
| (h) | (nM) | (nM) | Ratio | (nM) | (nM) | Ratio | (nM) | (nM) | Ratio |
| 0.063 | 59527 ± 3505 | 259861 ± 21561 | 0.28 | 41663 ± 8778 | 211920 ± 22922 | 0.20 | BLOQ | 168 ± 98.52 | NA |
| 0.25 | 123222 ± 49748 | 376314 ± 182744 | 0.33 | 86561 ± 10358 | 311591 ± 40730 | 0.28 | BLOQ | 345 ± 101 | NA |
| 0.5 | 83539 ± 43505 | 370863 ± 119965 | 0.23 | 47410 ± 24518 | 389162 ± 65991 | 0.38 | 12.1 ± 0.53 | 555 ± 93.4 | 0.02 |
| 1 | 153682 ± 38761 | 357779 ± 86447 | 0.46 | 102567 ± 16241 | 205115 ± 27325 | 0.50 | 14.10 ± 7.30 | 961 ± 189 | 0.01 |
| 2 | 96544 ± 15254 | 552023 ± 309077 | 0.15 | 44776 ± 3162 [symbol] | 80639 ± 5808 | 0.56 | 44.9 ± 20.08 | 1610 ± 968 | 0.03 |
| 4 | 12750 ± 2849 | 27155 ± 4732 | 0.47 | 4739 ± 585 | 10345 ± 724 | 0.45 | 25.4 ± 6.52 | 1110 ± 250 | 0.02 |
| 8 | 5765 ± 3855 | 10048 ± 6715 | 0.57 | 1236 ± 654 | 3295 ± 1528 | 0.38 | BLOQ | 314 ± 185 | NA |
| 24 | 914 ± 532 | 1581 ± 509 | 0.60 | 122 ± 4 | 10009 ± 5943 | 0.01 | BLOQ | BLOQ | NA |
| | | | | | | | Lynparza + LMP400 (10 mg/kg) | | |
| | | | | | | | | | Brain/ |
| | | | | | | | Brain | Plasma | Plasma |
| | | | | | | Time | Conc | Conc | Conc |
| | | | | | | (h) | (nM) | (nM) | Ratio |
| | | | | | | 0.063 | 5.80 ± 0.02 | 282 ± 58.4 | 0.02 |
| | | | | | | 0.25 | 13.1 ± 6.99 | 506 ± 423 | 0.02 |
| | | | | | | 0.5 | 33.36 ± 18.87 | 1384 ± 929 | 0.02 |
| | | | | | | 1 | 50.03 ± 9.79 | 2644 ± 762 | 0.02 |
| | | | | | | 2 | 30.44 ± 15.89 | 1637 ± 1051 | 0.02 |
| | | | | | | 4 | 18.39 ± 8.59 | 755 ± 460 | 0.02 |
| | | | | | | 8 | BLOQ | 166 ± 208 | NA |
| | | | | | | 24 | BLOQ | BLOQ | NA |

[symbol]

Three animals were used in the determination of the data per time point for each compound, for a total of 96 animals used to generate the data the table. [symbol]
indicates data missing or illegible when filed

TABLE 15a
Plasma Pharmacokinetic Parameters Observed
After IV Administration of 20 mg/kg of G103,
G104, G105, and LMP744 to Male CD1 Mice^{a}

| | | G103 | G104 | G105 | LMP744 |
|---|---|---|---|---|---|
| PK parameters | Units | Mean^{b} | Mean | Mean | Mean |
| Cl_obs | mL/min/ | — | 144 | 113 | 156 |
| | kg | | | | |
| T_{1/2} | h | — | 7.06 | 6.18 | 7.48 |
| C₀ | ng/mL | 544 | 1164 | 3904 | 1943 |
| AUCₗₐₛₜ | h*ng/mL | 2927 | 2160 | 2847 | 2022 |
| AUC_{Inf} | h*ng/mL | — | 2316 | 2949 | 2132 |
| AUC_{—}_{% Extrap}_{_{—}}obs | % | 21.0 | 6.77 | 3.48 | 5.16 |
| MRT_{Inf}_{_{—}}obs | h | — | 6.77 | 4.12 | 5.14 |
| AUCₗₐₛₜ/D | h*mg/mL | 146 | 108 | 142 | 101 |
| Vₛₛ_{_{—}}obs | L/kg | — | 58.4 | 29 | 48.2 |

^{a}The PK parameters were estimated by a non-compartmental model using WinNonlin software.
^{b}It was not possible to calculate meaningful Cl_obs, T_{1/2}, AUC_{Inf}, MRT_{Inf}_{_{—}}obs, and Vₛₛ_{_{—}}obs parameters for G103 because the AUC_{—}_{% Extrap}_{_{—}}obs was greater than 20%.

TABLE 15b
Plasma Pharmacokinetic Parameters for LMP400 and LMP744 Observed
After IV Administration of 10 mg/kg of LMP400 and 10 mg/kg
or 20 mg/kg of LMP744 in Combination with PO Administration of
100 mg/lg of Lynparza or 125 mg/kg of Temador to Mice^{a}

| | | LMP400 + | LMP400 + | LMP744 + | LMP744 + |
|---|---|---|---|---|---|
| PK parameters | Unit | Lynparza^{b} | Temador^{c} | Lynparza^{d} | Temador^{e} |
| Cl_obs | mL/min/kg | 23.3 | 26.7 | 109 | 116 |
| T_{1/2} | h | 6.74 | 7.03 | 5.43 | 5.20 |
| C₀ | ng/ml | 2286 | 3042 | 2072 | 4973 |
| AUCₗₐₛₜ | h*ng/ml | 6642 | 5823 | 1491 | 2813 |
| AUC_{Inf} | h*ng/ml | 7144 | 6248 | 1535 | 2881 |
| AUC_{—}_{% Extrap}_{_{—}}obs | % | 7.02 | 6.80 | 2.83 | 2.37 |
| MRT_{Inf}_{_{—}}obs | h | 7.44 | 7.41 | 4.33 | 4.09 |
| AUCₗₐₛₜ/D | h*mg/mL | 664 | 582 | 149 | 141 |
| Vₛₛ_{_{—}}obs | L/kg | 10.4 | 11.9 | 28.2 | 28.4 |

^{a}The PK parameters were estimated by a non-compartmental model using WinNonlin software.
^{b}IV administration of 10 mg/kg LMP400 plus PO administration of 100 mg/kg of Lynparza.
^{c}IV administration of 10 mg/kg of LMP400 plus PO administration of 125 mg/kg of Temador.
^{d}IV administration of 10 mg/kg of LMP744 plus PO administration of 100 mg/kg of Lynparza.
^{e}IV administration of 20 mg/kg of LMP744 plus 125 mg/kg of Temador.

TABLE 16a
Brain Pharmacokinetic Parameters Observed After IV Administration
of 20 mg/kg of G103, G104, G105, and LMP744 to Male CD1 Mice^{a}

| PK parameters | Unit | G103 Mean | G104 Mean | G105 Mean | LMP744 Mean |
|---|---|---|---|---|---|
| Tₘₐₓ | h | 0.083 | 0.083 | 0.25 | 0.25 |
| Cₘₐₓ | ng/g | 3809 | 1613 | 435 | 413 |
| AUCₗₐₛₜ | h*ng/g | 11690 | 10293 | 9698 | 7606 |
| AUC_{—}_{% Extrap}_{_{—}}obs | % | 46.8 | 46.1 | 99.2 | 72 |
| AUCₗₐₛₜ/D | h*mg/g | 584 | 514 | 485 | 380 |

^{a}The PK parameters were estimated by a non-compartmental model using WinNonlin software. It was not possible to calculate meaningful T_{1/2}, AUC_{Inf}, and MRT_{Inf}_{_{—}}obs parameters because the AUC_{—}_{% Extrap}_{_{—}}obs values were over 20%.

TABLE 16b
Brain Pharmacokinetic Parameters for LMP400 and LMP744 Observed
After IV Administration of 10 mg/kg of LMP400 and 10 mg/kg
or 20 mg/kg of LMP744 in Combination with PO Administration of
100 mg/lg of Lynparza or 125 mg/kg of Temador to Mice^{a}

| | | LMP400 + | LMP400 + | LMP744 + | LMP744 + |
|---|---|---|---|---|---|
| PK parameters | Unit | Lynparza^{b} | Temador^{c} | Lynparza^{d} | Temador^{e} |
| T_{1/2} | h | 6.01 | 6.35 | NA | NA |
| Tₘₐₓ | h | 0.0830 | 0.0830 | 2.00 | 1.00 |
| Cₘₐₓ | ng/g | 12173 | 9080 | 373 | 611 |
| AUCₗₐₛₜ | h*ng/g | 19983 | 15811 | 5860 | 12185 |
| AUC_{Inf} | h*ng/g | 20928 | 16590 | NA | NA |
| AUC_{—}_{% Extrap}_{_{—}}obs | % | 4.51 | 4.70 | NA | NA |
| MRT_{Inf}_{_{—}}obs | h | 5.68 | 5.67 | NA | NA |
| AUCₗₐₛₜ/D | h*mg/g | 1998 | 1581 | 186 | 609 |
| F | % | NA | NA | NA | NA |

^{a}The PK parameters were estimated by a non-compartmental model using WinNonlin software.
^{b}IV administration of 10 mg/kg LMP400 plus PO administration of 100 mg/kg of Lynparza.
^{c}IV administration of 10 mg/kg of LMP400 plus PO administration of 125 mg/kg of Temador.
^{d}IV administration of 10 mg/kg of LMP744 plus PO administration of 100 mg/kg of Lynparza.
^{e}IV administration of 20 mg/kg of LMP744 plus 125 mg/kg of Temador.

Tumor Penetration Studies. In addition to brain/plasma ratios, the tumor/plasma ratios of JQ1, G103, G104, and G105 were determined in mice bearing MOLT-4, RS4;11, and RD-ES tumor xenografts (Table 16). The ratios in the MOLT-4 and RD4;11 tumors were determined at the 3- and 9-hour time points, while the ratios in RD-ES were measured at the 3- and 6-hour time points. The “G compounds” were all administered by IV at the maximum tolerated dose level as well as half the maximum tolerated dose level, while experiments with JQ1 involved the standard IP administration of 50 mg/kg. The results reported in Table 16 reveal that all of the “G compounds” were highly concentrated in tumor tissue, while the tumor-plasma ratios of JQ1 were significantly lower. These results are consistent with those previously reported for the related indenoisoquinoline LMP744 in dogs, which documented extensive distribution in tumor tissue, with mean tumor concentrations being at least 100 times higher than the plasma concentrations at the same time points.⁴² Related studies of LMP400 in BALB/c female mice with CT26 colon tumors also demonstrated concentration in tumor tissue relative to plasma, although the ratios were lower (5 to 14.6 after IV administration).⁴⁰ Except for G104 (30 mg/kg) and G105 (10 mg/kg) vs. RE-ES, the ratios determined at the longer time point were larger than those determined at the shorter time point.

TABLE 16
Mean Tumor/Plasma Ratios in Tumor-Bearing Female NOD SCOD Mice after IV Administration

| Time (h) | JQ1 | G103 | G103 | G104 | G104 | G105 | G105 |
|---|---|---|---|---|---|---|---|
| (Tumor) | (50 mg/kg) | (50 mg/kg) | (25 mg/kg) | (30 mg/kg) | (15 mg/kg) | (20 mg/kg) | (10 mg/kg) |
| 3 (MOLT-4) | 4.99 ± 2.14 | 97 ± 22 | 89 ± 21 | 69.5 ± 8.8 | 51.3 ± 6.7 | 312 ± 500 | 50 ± 13.4 |
| 9 (MOLT-4) | 17.6 ± 19.5 | 131 ± 63 | 95 ± 21 | 106 ± 21 | 125 ± 29 | 326 ± 102 | 357 ± 180 |
| 3 (RS4:11) | 2.94 ± 0.78 | 221 ± 37 | 140 ± 89 | 77.3 ± 11.8 | 76.1 ± 18.9 | 111 ± 18 | 62.3 ± 17.7 |
| 9 (RS4:11 | 3.51 ± 1.60 | 270 ± 127 | 183 ± 46 | 112 ± 16 | 130 ± 32 | 383 ± 183 | 185 ± 25 |
| 3 (RD-ES) | 1.89 ± 0.65 | 220 ± 47 | 96 ± 53 | 91 ± 47 | 56 ± 31 | 174 ± 67 | 125 ± 64 |
| 6 (RD-ES) | 4.64 ± 1.12 | 307 ± 268 | 104 ± 21 | 90 ± 32 | 83 ± 26 | 279 ± 36 | 105 ± 55 |

The c-Myc and γ-H2AX expression levels were determined by western blotting in RS4;11, MOLT-4, and RD-ES xenografts after administration of JQ1, G103, G104, and G105 to female NOD SCID mice, and the results are listed in Tables 17-19. Excluding JQ1, the largest reduction in c-Myc expression was observed at the 9-hour time point after administration of G103 to mice harboring the RS4;11 xenograft, and the effect is greater at the higher dose level (Table 17). G103 also produced relatively large reductions in c-Myc in MOLT-4 xenografts, although the effect was not as great as that observed in RS4;11 cells (Table 18). Administration of G103 actually resulted in unexpected increases in levels of the c-Myc protein in RD-ES xenografts, and the effect is greater at higher dose levels (Table 19). Several moderate increases in c-Myc levels were observed in RS4;11 and MOLT-4 xenografts, but they are not as great or as prevalent as seen in RD-ES tumors (Table 19), where large increases were usually observed 3 hours after drug administration, usually followed by decreases seen at the 6-hour time point (the exception being G103 at the 25 mg/kg dose level). With G104 and G105 vs. RD-ES xenografts, the c-Myc levels were depressed below the control value at the 6-hour time point, but with G103 they were not (Table 19). In several cases, the levels of c-Myc were more greatly depressed at the lower dose level. For examples, see the 9-hour data for G104 in Tables 17 and 18 and the 6-hour data in Table 19. It is noteworthy that at the 3-hour time point in RD-ES cells, there is consistently more c-Myc expression at the high dose than at the low dose, and the levels at the high dose are significantly greater than the control. The greatest reduction in c-Myc was seen with JQ1 in MOLT-4 cells at the 3-hour time point (Table 18), although it produced large increases in c-Myc at the 3-hour time point in RD-ES xenograft (Table 19). Overall, there are many unexpected and unexplained aspects of the data in Tables 17-19, which are highly variable and also highly dependent on cell type. In particular, drug-induced increases in c-Myc expression relative to control and the larger changes in c-Myc expression seen at lower vs. higher doses are counterintuitive and unexplained.

The 7-azaindenoisoquinolines are known to induce histone H2AX phosphorylation of Ser139 (γ-H2AX) by causing DNA double-strand breaks.²⁵ As shown in Tables 17-19, G103, G104, and G105 consistently increased γ-H2AX production relative to control at the longer time point, with the one exception being G104 in RD-ES cells. The extent of DNA damage was generally higher at the longer time point, with the exceptions being with the lower dose of G104 and both doses of G105 in RD-ES cells (Table 19), as well as both doses of G103 in the RS4;11 xenograft (Table 17). There were numerous instances of γ-H2AX being depressed below control levels, and they generally occurred at the 3-hour time point, with the exception being JQ1 in RD-ES xenograft, where levels were depressed at both time points. (Table 19).

Of the three compounds (G103-G105) tested in more detail, G103 has the longest IV half-life, the greatest oral absorption, the greatest stability in human liver microsomes, the lowest efflux ratio in Caco-2 cells, the highest maximum tolerated dose in NOD SCID mice, and the greatest brain penetration. However, G105 appears to be the most effective of the three compounds as an anticancer agent in vivo.

TABLE 17
γ-H2AX and c-Myc Expression in RS4; 11 Tumor
Tissue Xenografts in Female NOD SCID Mice after
IP Administration of JQ1 and IV Administration
of G103-105 (Mean ± SEM % Relative to Vehicle)

| | Time | | |
|---|---|---|---|
| Group | point^{a} | γ-H2AX/GAPDH | c-Myc/GAPDH |
| Group 1: Vehicle control | 3 h | 100.00 ± 22.62 | 100.00 ± 3.82 |
| | 9 h | 100.00 ± 40.37 | 100.00 ± 5.37 |
| | 3 h | 100.00 ± 30.42 | 100.00 ± 3.30 |
| | 9 h | 100.00 ± 30.52 | 100.00 ± 5.75 |
| Group 2: JQ1 50 mg/kg | 3 h | 215.99 ± 65.53 | 95.66 ± 11.33 |
| | 9 h | 488.55 ± 90.39 | 103.46 ± 11.60 |
| Group 3: G103 50 mg/kg | 3 h | 348.75 ± 204.19^{b} | 117.59 ± 10.15 |
| | 9 h | 156.61 ± 28.68 | 55.78 ± 4.99 |
| Group 4: G103 25 mg/kg | 3 h | 143.13 ± 34.29 | 101.88 ± 10.08 |
| | 9 h | 112.64 ± 9.71 | 64.05 ± 5.82 |
| Group 5: G104 30 mg/kg | 3 h | 150.28 ± 29.86 | 111.60 ± 8.19 |
| | 9 h | 220.37 ± 47.79 | 100.31 ± 2.71 |
| Group 6: G104 15 mg/kg | 3 h | 133.08 ± 38.59 | 92.20 ± 2.11 |
| | 9 h | 142.66 ± 23.92 | 92.59 ± 13.64 |
| Group 7: G105 20 mg/kg | 3 h | 219.29 ± 82.49 | 100.30 ± 16.76 |
| | 9 h | 540.94 ± 97.90 | 80.45 ± 3.93 |
| Group 8: G105 10 mg/kg | 3 h | 67.00 ± 2.90 | 102.74 ± 3.45 |
| | 9 h | 166.37 ± 40.45 | 74.98 ± 3.52 |

^{a}Time after administration of the vehicle or the drug.
^{b}This value was obtained after excluding an outlier of 4115.66.

TABLE 18
γ-H2AX and c-Myc Expression in MOLT-4 Tumor
Tissue Xenografts in Female NOD SCID Mice after
IP Administration of JQ1 and IV Administration
of G103-105 (Mean ± SEM % Relative to Vehicle)

| | Time | | |
|---|---|---|---|
| Group | point^{a} | γ-H2AX/GAPDH | c-Myc/GAPDH |
| Group 1: Vehicle control | 3 h | 100.00 ± 28.82 | 100.00 ± 5.11 |
| | | 100.00 ± 25.27 | 100.00 ± 7.33 |
| | 9 h | 100.00 ± 11.60 | 100.00 ± 6.13 |
| | | 100.00 ± 16.33 | 100.00 ± 4.66 |
| Group 2: JQ1 50 mg/kg | 3 h | 100.52 ± 12.94 | 43.97 ± 5.50 |
| | 9 h | 195.71 ± 37.32 | 78.79 ± 10.88 |
| Group 3: G103 50 mg/kg | 3 h | 83.88 ± 7.09 | 98.83 ± 3.21 |
| | 9 h | 154.28 ± 23.45 | 80.72 ± 3.00 |
| Group 4: G103 25 mg/kg | 3 h | 102.05 ± 21.92 | 82.82 ± 5.78 |
| | 9 h | 129.33 ± 20.84 | 86.69 ± 5.00 |
| Group 5: G104 30 mg/kg | 3 h | 47.29 ± 12.20 | 114.32 ± 4.41 |
| | 9 h | 168.88 ± 39.58 | 100.33 ± 2.35 |
| Group 6: G104 15 mg/kg | 3 h | 71.86 ± 8.95 | 119.75 ± 13.40 |
| | 9 h | 138.12 ± 23.51 | 88.54 ± 7.63 |
| Group 7: G105 20 mg/kg | 3 h | 56.62 ± 11.87 | 96.74 ± 9.71 |
| | 9 h | 109.66 ± 28.34 | 83.89 ± 10.06 |
| Group 8: G105 10 mg/kg | 3 h | 60.98 ± 8.93 | 110.56 ± 4.90 |
| | 9 h | 172.03 ± 47.41 | 84.53 ± 6.26 |

^{a}Time after administration of the vehicle or the drug.

TABLE 19
γ-H2AX and c-Myc Expression in RD-ES Tumor Tissue Xenografts
in Female NOD SCID Mice after IP Administration of JQ1 and IV
Administration of G103-105 (Mean ± SEM % Relative to Vehicle)

| | Time | | |
|---|---|---|---|
| Group | point^{a} | γ-H2AX/GAPDH | c-Myc/GAPDH |
| Group 1: Vehicle control | 3 h | 100.00 ± 24.39 | 100.00 ± 20.77 |
| | 6 h | 100.00 ± 21.54 | 100.00 ± 16.62 |
| | 3 h | 100.00 ± 26.45 | 100.00 ± 25.70 |
| | 6 h | 100.00 ± 14.59 | 100.00 ± 17.94 |
| Group 2: JQ1 50 mg/kg | 3 h | 45.58 ± 9.62 | 160.32 ± 10.20 |
| | 6 h | 68.63 ± 15.29 | 115.33 ± 23.01 |
| Group 3: G103 50 mg/kg | 3 h | 86.78 ± 9.41 | 198.20 ± 28.95 |
| | 6 h | 188.94 ± 49.17 | 121.62 ± 21.79 |
| Group 4: G103 25 mg/kg | 3 h | 122.01 ± 22.44 | 108.74 ± 14.51 |
| | 6 h | 126.77 ± 26.86 | 117.57 ± 14.72 |
| Group 5: G104 30 mg/kg | 3 h | 85.61 ± 23.26 | 188.78 ± 28.24 |
| | 6 h | 99.26 ± 10.19 | 91.67 ± 23.70 |
| Group 6: G104 15 mg/kg | 3 h | 155.18 ± 11.44 | 128.94 ± 6.14 |
| | 6 h | 101.89 ± 7.35 | 53.57 ± 10.75 |
| Group 7: G105 20 mg/kg | 3 h | 134.62 ± 18.03 | 154.98 ± 17.80 |
| | 6 h | 109.81 ± 27.72 | 71.97 ± 14.55 |
| Group 8: G105 10 mg/kg | 3 h | 145.80 ± 32.79 | 128.18 ± 13.67 |
| | 6 h | 111.73 ± 14.44 | 69.45 ± 15.02 |

^{a}Time after administration of the vehicle or the drug.

### Experimental Section

5-Bromopyridine-2,3-diol (7). A cold aqueous solution was prepared by adding furaldehyde (5, 20.10 g, 17.3 mL, 209.2 mmol, 1 equiv) from a freshly opened container to ice (200 g). The solution was vigorously stirred and bromine (33.43 g, 10.78 mL, 209.2 mmol, 1 equiv) was added dropwise at such a rate as to maintain the reaction temperature at 0° C. (using an external ice-bath). The reaction mixture was stirred for 30 min. Concentrated HCl (10 mL) was added in one portion and the reaction mixture was stirred for 30 min at 0° C. Additional bromine (33.43 g, 10.78 mL, 209.2 mmol, 1 equiv) was added dropwise over a period of 1 h while maintaining the reaction temperature at −5 to 0° C. The reaction mixture was then filtered and sulfamic acid (6) (20.31 g, 209.2 mmol, 1 equiv) was added to the filtrate and the mixture was stirred vigorously for 3 h at 50° C. The reaction mixture was cooled to 0° C. and the resulting solid precipitate was collected by filtration. The solid was dried for 22 h at 25° C. to afford 5-bromopyridine-2,3-diol (7, 19.8 g, 104 mmol, 49.8%) as a brown solid. ¹H NMR (300 MHz, DMSO-d₆) δ 11.87 (s, 1H), 9.69 (s, 1H), 7.09 (d, J=2.5 Hz, 1H), 6.79 (d, J=2.5 Hz, 1H).

6-Bromo-[1,3]dioxolo[4,5-b]pyridine (8). Dibromomethane (14.6 g, 5.88 mL, 84.2 mmol, 2.0 equiv), potassium carbonate (11.6 g, 84.2 mmol, 2.0 equiv) and copper(II) oxide (1.0 g, 12.6 mmol, 0.30 equiv) were added to a solution of 5-bromopyridine-2,3-diol (3, 8.0 g, 42.1 mmol, 1 equiv) in DMF (80 mL) and the reaction mixture was stirred and heated to 90° C. for 14 h. The reaction mixture was cooled to room temperature, filtered through a Celite bed and the bed was washed with ethyl acetate (2×100 mL). The filtrate was extracted with ethyl acetate (2×200 mL). The combined ethyl acetate extract was washed with water (100 mL) and then with brine (100 mL) and dried with anhydrous MgSO4. The solvent was evaporated to dryness to provide the crude product, which was purified by flash column chromatography [silica gel (40 g), eluting with 0-100% ethyl acetate in hexanes] to afford 6-bromo-[1,3]dioxolo[4,5-b]pyridine (8, 1.0 g, 4.95 mmol, 12%) as a white solid. ¹H NMR (300 MHz, DMSO-d₆) δ 7.71 (d, J=2.0 Hz, 1H), 7.55 (dd, J=2.0, 0.9 Hz, 1H), 6.20 (s, 2H).

6-Methyl-[1,3]dioxolo[4,5-b]pyridine (9). A mixture of 6-bromo-[1,3]dioxolo[4,5-b]pyridine (8, 10.0 g, 49.5 mmol, 1 equiv), methylboronic acid (5.93 g, 99.0 mmol, 2 equiv), sodium carbonate (21.0 g, 198 mmol, 4.0 equiv), 2-dicyclohexylphosphino-2′,6′-diisopropoxybiphenyl (RuPHOS) (2.31 g, 4.95 mmol, 0.1 equiv), and Pd₂dba₃ (2.27 g, 2.48 mmol, 0.050 equiv) in toluene (200 mL) and water (20 mL) was degassed and then filled with nitrogen. The reaction mixture was heated to 110° C. for 12 h and was then cooled to room temperature and diluted with ethyl acetate (100 mL) and water (50 mL). The reaction mixture was stirred and then allowed to stand to separate the layers. The aqueous layer was back-extracted with ethyl acetate (100 mL). The combined organic extract was dried over anhydrous MgSO₄, filtered and evaporated to dryness. The remaining residue was purified twice by flash column chromatography [silica gel (2×120 g), eluting with 0-100% ethyl acetate in hexanes] to afford 6-methyl-[1,3]dioxolo[4,5-b]pyridine (9, 5.6 g, 41 mmol, 82%) as a thick yellow syrup. ¹H NMR (300 MHz, DMSO-d₆) δ 7.45-7.34 (m, 1H), 7.10 (d, J=1.8 Hz, 1H), 6.07 (s, 2H), 2.18 (s, 3H).

5-Bromo-6-methyl-[1,3]dioxolo[4,5-b]pyridine (10). N-Bromosuccinimide (10.9 g, 61.0 mmol, 1.1 equiv) was added to a stirred solution of 6-methyl-[1,3]dioxolo[4,5-b]pyridine (9) (7.60 g, 55.4 mmol, 1 equiv) and acetic acid (1.14 mL) in acetonitrile (150 mL) at room temperature. The reaction mixture was stirred at room temperature for 3 h. The reaction mixture was diluted with water (300 mL) and the product was extracted with ethyl acetate (2×300 mL). The combined ethyl acetate extract was dried over anhydrous MgSO₄, filtered and the solvent was evaporated to dryness. The remaining residue was purified twice by flash column chromatography [silica gel (2×120 g), eluting with 0-100% ethyl acetate in hexanes] to furnish 5-bromo-6-methyl-[1,3]dioxolo[4,5-b]pyridine (10; 6.5 g, 30 mmol, 54%) as a light reddish solid. ¹H NMR (300 MHz, DMSO-d₆) δ 7.32 (s, 1H), 6.17 (s, 2H), 2.22 (s, 3H).

6-Methyl-[1,3]dioxolo[4,5-b]pyridine-5-carbonitrile (11). CuCN (5.4 g, 60 mmol, 2.0 equiv) was added to a stirred solution of 5-bromo-6-methyl-[1,3]dioxolo[4,5-b]pyridine (10, 6.5 g, 30 mmol, 1 equiv) in dry DMF (40 mL) and toluene (80 mL). The reaction mixture was stirred and heated at 110° C. for 18 h. After completion of the reaction, brine (300 mL) was added, and the product was extracted with ethyl acetate (2×500 mL). The combined extract was dried over anhydrous MgSO₄, filtered and the solvent was evaporated. The remaining residue was purified by flash column chromatography [silica gel (120 g), eluting with 0-100% of a 9:1 mixture of ethyl acetate/methanol in hexanes] to furnish 6-methyl-[1,3]dioxolo[4,5-b]pyridine-5-carbonitrile (11, 2.3 g, 19 mmol, 47%) as a white solid. ¹H NMR (300 MHz, DMSO-d₆) δ 7.35 (s, 1H), 6.26 (s, 2H), 2.38 (s, 3H).

6-(Bromomethyl)-[1,3]dioxolo[4,5-b]pyridine-5-carbonitrile (12). N-Bromosuccinimide (1.0 g, 5.61 mmol, 1.30 equiv) and benzoyl peroxide (83.7 mg, 75% wt, 259 μmol, 0.060 equiv) were added to a stirred solution of 6-methyl-[1,3]dioxolo[4,5-b]pyridine-5-carbonitrile (11, 0.70 g, 4.32 mmol, 1.0 equiv) in carbon tetrachloride (35 mL) at room temperature. The mixture was heated at reflux for 16 h. The reaction mixture was concentrated and the residue was purified by flash column chromatography [silica gel (12 g), eluting with 0-100% ethyl acetate in hexanes] to furnish 6-(bromomethyl)-[1,3]dioxolo[4,5-b]pyridine-5-carbonitrile (12, 0.488 g, 2.02 mmol, 46.9%) as a white solid. ¹H NMR (300 MHz, DMSO-d₆) δ 7.54 (s, 1H), 6.31 (s, 2H), 4.71 (s, 2H).

2,3-Dimethoxy-6,12-dihydro-5H-[1,3]dioxolo[4″,5″:5′,6′]pyrido[3′,2′:4,5]cyclopenta[1,2-c]isoquinolin-5-one (14). 6,7-Dimethoxyisochromane-1,3-dione (13, 409 mg, 1.84 mmol, 1.2 equiv) was added to 6-(bromomethyl)[1,3]dioxolo[4,5-b]pyridine-5-carbonitrile (12, 0.370 g, 1.54 mmol, 1 equiv) in acetonitrile (22 mL, 60 vol equiv). Triethylamine (388 mg, 535 μL, 3.84 mmol, 2.50 equiv) was then added and the mixture became a clear solution. The reaction mixture was heated at reflux for 15 h and was then cooled to room temperature, and the precipitated solid was collected by filtration and rinsed with acetonitrile to afford 2,3-dimethoxy-6,12-dihydro-5H-[1,3]dioxolo[4″,5″:5′,6′]pyrido[3′,2′:4,5]cyclopenta[1,2-c]isoquinolin-5-one (14, 0.310 g, 916 μmol, 59.7%) as a light yellow solid. ¹H NMR (300 MHz, DMSO-d₆) δ 11.84 (s, 1H), 7.61 (s, 1H), 7.54 (s, 1H), 7.13 (s, 1H), 6.21 (s, 2H), 3.95 (s, 3H), 3.87 (s, 3H), 3.76 (s, 2H).

2,3-Dimethoxy-5H-[1,3]dioxolo[4″,5″:5′,6′]pyrido[3′,2′:4,5]cyclopenta[1,2-c]isoquinoline-5,12(6H)-dione (15). A mixture of 2,3-dimethoxy-6,12-dihydro-5H-[1,3]dioxolo[4″,5″:5′,6′]pyrido[3′,2′:4,5]cyclopenta[1,2-c]isoquinolin-5-one (14, 50 mg, 0.15 mmol, 1 equiv) and selenium dioxide (66 mg, 0.59 mmol, 4.0 equiv) in dioxane (7 mL, 140 vol. equiv) and acetic acid (8.9 mg, 8.5 μL, 0.15 mmol, 1 equiv) was heated at reflux for 11 h. The reaction mixture was filtered and the obtained solid was rinsed with dioxane to afford 2,3-dimethoxy-5H-[1,3]dioxolo[4″,5″:5′,6′]pyrido[3′,2′:4,5]cyclopenta[1,2-c]isoquinoline-5,12(6H)-dione (15, 0.041 g, 0.12 mmol, 79%) as a brown solid. ¹H NMR (300 MHz, DMSO-d₆) δ 7.67 (s, 1H), 7.50 (s, 1H), 7.35 (s, 1H), 6.28 (s, 2H), 3.90 (s, 3H), 3.86 (s, 3H).

2,3-Dimethoxy-6-(3-morpholinopropyl)-5H-[1,3]dioxolo[4″,5″:5′,6′]pyrido[3′,2′:4,5]cyclopenta[1,2-c]isoquinoline-5,12(6H)-dione (17). NaH (12 mg, 60% wt, 0.30 mmol, 3.0 equiv) and KI (1.6 mg, 0.1 equiv) were added to a suspension of 2,3-dimethoxy-5H-[1,3]dioxolo[4″,5″:5′,6′]pyrido[3′,2′:4,5]cyclopenta[1,2-c]isoquinoline-5,12(6H)-dione (15, 0.035 g, 1 equiv) in dry DMF (3 mL) at 5° C. The reaction mixture was warmed to room temperature and stirred for 1 h to form a dark-red solution. The solution was cooled to 0° C., and 4-(3-bromopropyl)morpholine hydrobromide (16, 37 mg, 0.13 mmol, 1.3 equiv) was added and the solution was stirred at room temperature for 12 h. The reaction was quenched with water (100 mL) and the product was extracted with CH₂Cl₂ (2×100 mL) and the CH₂Cl₂ extract was washed with brine (100 mL), dried with anhydrous MgSO₄, and the solvent was evaporated to dryness. The remaining residue was purified by flash column chromatography [silica gel (24 g), eluting with 0-50% methanol in CH₂Cl₂] to furnish 2,3-dimethoxy-6-(3-morpholinopropyl)-5H-[1,3]dioxolo[4″,5″:5′,6′]pyrido [3′,2′:4,5]cyclopenta[1,2-c]isoquinoline-5,12(6H)-dione (17, 0.018 g, 38 μmol, 38%) as a dark brown solid. ¹H NMR (300 MHz, CDCl₃) δ 7.94 (s, 1H), 7.68 (s, 1H), 7.17 (s, 1H), 6.18 (s, 2H), 4.88 (t, J=7.6 Hz, 2H), 4.05 (s, 3H), 3.99 (s, 3H), 3.70-3.55 (m, 4H), 2.56 (t, J=6.6 Hz, 2H), 2.46 (s, 4H), 2.01 (s, 2H); MS (ES+): 480.4.

Alternate Synthesis of 17. DIPEA (0.15 g, 0.20 mL, 1.2 mmol, 10 equiv) and morpholine (0.20 g, 0.20 mL, 2.3 mmol, 20 equiv) were added to a stirred solution of 6-(3-bromopropyl)-2,3-dimethoxy-5H-[1,3]dioxolo[4″,5″:5′,6′]pyrido[3′,2′:4,5]cyclopenta[1,2-c]isoquinoline-5,12(6H)-dione (20, 55 mg, 0.12 mmol, 1 equiv) in DMF (11 mL). The reaction mixture was stirred for 12 h at room temperature and then heated to 50° C. for 12 h. The solvent was evaporated and the residue was triturated with water to form a solid. The solid was filtered and rinsed with water to afford 2,3-dimethoxy-6-(3-morpholinopropyl)-5H-[1,3]dioxolo[4″,5″:5′,6′]pyrido[3′,2′:4,5]cyclopenta[1,2-c]isoquinoline-5,12(6H)-dione (17, 24 mg, 50 μmol, 43%) as a purple solid that was treated with water/acetonitrile (0.8 mL/0.2 mL) followed by 5 equiv of 4N HCl and then soaked for few minutes and lyophilized to dryness to afford the HCl salt of 17 (26 mg, 42%). ¹H NMR (300 MHz, CDCl₃) δ 7.94 (s, 1H), 7.68 (s, 1H), 7.16 (s, 1H), 6.18 (s, 2H), 5.02-4.80 (m, 2H), 4.05 (s, 3H), 4.00 (s, 3H), 3.66 (s, 4H), 2.64-2.53 (m, 2H), 2.46 (s, 4H), 2.11-1.93 (m, 2H); MS (ES+): 480.4 (M+1).

6-(3-(1H-Imidazol-1-yl)propyl)-2,3-dimethoxy-5H-[1,3]dioxolo[4″,5″:5′,6′]pyrido[3′,2′:4,5]cyclopenta[1,2-c]isoquinoline-5,12(6H)-dione (18). NaH (28 mg, 60 wt. %, 0.71 mmol, 5.0 equiv) was added to a suspension of 2,3-dimethoxy-5H-[1,3]dioxolo[4″,5″:5′,6′]pyrido[3′,2′:4,5]cyclopenta[1,2-c]isoquinoline-5,12(6H)-dione (15, 50 mg, 0.14 mmol, 1 equiv) and 1-(3-bromopropyl)-1H-imidazole (95 mg, 0.35 mmol, 2.5 equiv) in dry DMF (10 mL) at room temperature. The reaction mixture was stirred for 3 h. The reaction mixture was quenched with ice-cold water and extracted with CH₂Cl₂. A turbid solution was formed which was filtered using a Whatman filter paper to obtain a gummy solid. The LCMS analysis indicated the presence of the desired substance in this crude product. The gummy solid was re-dissolved in methanol/CH₂Cl₂, mixed with silica gel (2 g) and the solvent was evaporated to form a slurry. The silica gel slurry was placed on the top of a column containing silica gel (24 g) and the product was eluted with 0-10% DMA80 (80% DCM, 18% methanol, and 2% ammonia by volume) in CH₂Cl₂ to furnish 6-(3-(1H-imidazol-1-yl)propyl)-2,3-dimethoxy-5H[1,3]dioxolo[4″,5″:5′,6′]pyrido[3′,2′:4,5]cyclopenta[1,2-c]isoquinoline-5,12(6H)-dione (18, 8 mg, 0.02 mmol, 10%) as a light brown solid. ¹H NMR (300 MHz, CDCl₃) δ 7.94 (s, 1H), 7.68 (s, 1H), 7.57 (s, 1H), 7.18 (s, 1H), 7.07 (s, 1H), 7.02 (s, 1H), 6.21 (s, 2H), 4.84 (t, J=7.3 Hz, 2H), 4.16 (t, J=7.5 Hz, 2H), 4.05 (s, 3H), 4.01 (s, 3H), 0.95-0.77 (m, 2H); MS (ES+): 461.

Alternate Synthesis of 18. 6-(3-Bromopropyl)-2,3-dimethoxy-5H-[1,3]dioxolo[4″,5″:5′,6′]pyrido[3′,2′:4,5]cyclopenta[1,2-c]isoquinoline-5,12(6H)-dione (20, 55 mg, 0.12 mmol, 1 equiv), DIPEA (0.15 g, 0.20 mL, 10 equiv, 1.2 mmol) and 1H-imidazole (0.16 g, 2.3 mmol, 20.0 equiv) were dissolved in DMF (11 mL). The reaction mixture was stirred for 16 h at room temperature. The reaction mixture was then heated to 70° C. for 18 h. The solvent was evaporated to dryness and the residue was treated with CH₂Cl₂ (200 mL) and brine (100 mL). The mixture was filtered, and the layers were separated. The aq layer was back-extracted with CH₂Cl₂ (100 mL) and the combined organic extract was dried over anhydrous MgSO₄. The solution was filtered, and the solvent was evaporated to dryness. The residue was purified by flash column chromatography [silica gel (24 g), eluting with 0-10% MeOH in CH₂Cl₂] to furnish 6-(3-(1H-imidazol-1-yl)propyl)-2,3-dimethoxy-5H-[1,3]dioxolo[4″,5″:5′,6′]pyrido[3′,2′:4,5]cyclopenta[1,2-c]isoquinoline-5,12(6H)-dione (18, 7 mg, 0.02 mmol, 10%) as a purple solid. The compound was converted to the HCl salt by treating with 7 equiv of 4N HCl in water/acetonitrile (8:2 ratio) followed by lyophilization to dryness to afford the HCl salt 18 (9.1 mg). ¹H NMR (300 MHz, DMSO-d₆) δ 9.14 (s, 1H), 7.84-7.81 (m, 1H), 7.80 (s, 1H), 7.71-7.66 (m, 1H), 7.52 (s, 1H), 7.43 (s, 1H), 6.30 (s, 2H), 4.69 (t, J=6.3 Hz, 2H), 4.32 (t, J=6.8 Hz, 2H), 3.92 (s, 3H), 3.88 (s, 3H), 2.40-2.25 (m, 2H); MS (ES+): 461.4 (M+1).

6-(3-(Isopropylamino)propyl)-2,3-dimethoxy-5H-[1,3]dioxolo [4″,5″:5′,6′]pyrido[3′,2′:4,5]cyclopenta[1,2-c]isoquinoline-5,12(6H)-dione (19). NaH (28 mg, 60 wt. %, 0.71 mmol, 5.0 equiv) was added to a suspension of 2,3-dimethoxy-5H-[1,3]dioxolo[4″,5″:5′,6′]pyrido[3′,2′:4,5]cyclopenta[1,2-c]isoquinoline-5,12(6H)-dione (15, 50 mg, 0.14 mmol, 1 equiv) in DMF (3 mL). 3-Bromo-N-isopropylpropan-1-amine hydrobromide (92 mg, 0.35 mmol, 2.5 equiv) in dry DMF (10 mL) was then added at room temperature and the reaction mixture was stirred for 3 h. The reaction mixture was quenched with ice-cold water and extracted with CH₂Cl₂. The CH₂Cl₂ layer was dried over anhydrous MgSO₄, filtered and rinsed with CH₂Cl₂ (50 mL). The solvent was evaporated to dryness and the obtained residue was purified by flash column chromatography [silica gel (24 g), eluting with 0-10% DMA80 (80% DCM, 18% methanol, and 2% ammonia by volume) in CH₂Cl₂] to furnish 6-(3-(isopropylamino)propyl)-2,3-dimethoxy-5H-[1,3]dioxolo[4″,5″:5′,6′]pyrido[3′,2′:4,5]cyclopenta[1,2-c]isoquinoline-5,12(6H)-dione (19, 3 mg, 6.6 μmol, 4.7%) as a brown solid. ¹H NMR (300 MHz, CDCl₃) δ 7.94 (s, 1H), 7.61 (s, 1H), 7.16 (s, 1H), 6.20 (s, 2H), 5.03-4.86 (m, 2H), 4.07 (s, 3H), 4.01 (s, 3H), 3.45-3.28 (m, 1H), 2.93 (s, 2H), 2.61 (s, 2H), 1.49 (d, J=6.5 Hz, 6H); MS (ES+): 452.52.

Alternate Synthesis of 19. 6-(3-Bromopropyl)-2,3-dimethoxy-5H-[1,3]dioxolo[4″,5″:5′,6′]pyrido[3′,2′:4,5]cyclopenta[1,2-c]isoquinoline-5,12(6H)-dione (20, 52 mg, 0.11 mmol, 1 equiv), DIPEA (0.14 g, 0.19 mL, 1.1 mmol, 10 equiv) and isopropylamine (0.13 g, 0.19 mL, 2.2 mmol, 20 equiv) were dissolved in DMF (11 mL). The reaction mixture was stirred for 12 h at room temperature. The reaction mixture was treated with brine (100 mL) and CH₂Cl₂ (100 mL) and stirred for 10 min. The layers were separated, and the aq layer was back-extracted with CH₂Cl₂ (100 mL). The combined organic extract was dried over anhydrous MgSO₄, filtered, and the solvent was evaporated to dryness. The residue was purified by flash column chromatography [silica gel (24 g), eluting with 0-20% methanol in CH₂Cl₂] to furnish the product (10 mg) in the form of the free base as a purple solid. The solid was treated with water (0.8 mL) and acetonitrile (0.2 mL) followed by 5 equiv of 4N aq HCl. The components were mixed for a few minutes and then lyophilized to dryness to afford 6-(3-(isopropylamino)propyl)-2,3-dimethoxy-5H-[1,3]dioxolo[4″,5″:5′,6′]pyrido[3′,2′:4,5]cyclopenta[1,2-c]isoquinoline-5,12(6H)-dione·2HCl (19, 5.8 mg, 13 μmol, 12%).¹H NMR (300 MHz, DMSO-d₆) δ 8.36 (s, 2H), 7.85 (s, 1H), 7.55 (s, 1H), 7.50 (s, 1H), 6.33 (s, 2H), 4.84-4.63 (m, 2H), 3.93 (s, 3H), 3.89 (s, 3H), 3.01 (s, 2H), 2.21-2.03 (m, 2H), 1.21 (d, J=6.5 Hz, 6H). MS (ES+): 452.5 (M+1).

Bromopropyl)-2,3-dimethoxy-5H-[1,3]dioxolo[4″,5″:5′,6′]pyrido[3′,2′:4,5]cyclopenta[1,2-c]isoquinoline-5,12(6H)-dione (20). NaH (54.5 mg, 60 wt. %, 1.36 mmol, 1.2 equiv) was added to a suspension of 2,3-dimethoxy-5H-[1,3]dioxolo[4″,5″:5′,6′]pyrido[3′,2′:4,5]cyclopenta[1,2-c]isoquinoline-5,12(6H)-dione (15, 400 mg, 1.14 mmol, 1 equiv) and KI (37.7 mg, 227 μmol, 0.2 equiv) in DMF (36 mL) at 0° C. and the reaction mixture was warmed to room temperature, stirred for 2 h, then cooled to 0° C. 1,3-Dibromopropane (275 mg, 139 μL, 1.36 mmol, 1.2 equiv) was added and the solution was warmed to room temperature and stirred for 20 h. The reaction mixture was diluted with water (120 mL), and the resulting solid was collected by filtration, rinsed with water (2×30 mL) and dried at room temperature to furnish 6-(3-bromopropyl)-2,3-dimethoxy-5H-[1,3]dioxolo[4″,5″:5′,6′]pyrido[3′,2′:4,5]cyclopenta[1,2-c]isoquinoline-5,12(6H)-dione (20, 0.256 g, 541 μmol, 47.6%) as a purple solid. ¹H NMR (300 MHz, CDCl₃) δ 7.94 (s, 1H), 7.69 (s, 1H), 7.17 (s, 1H), 6.19 (s, 2H), 4.94 (t, J=7.1 Hz, 2H), 4.05 (s, 3H), 4.00 (s, 3H), 3.56 (t, J=7.0 Hz, 2H), 2.43 (p, J=7.3 Hz, 2H).

6-(3-(Ethylamino)propyl)-2,3-dimethoxy-5H-[1,3]dioxolo[4″,5″:5′,6′]pyrido [3′,2′:4,5]cyclopenta[1,2-c]isoquinoline-5,12(6H)-dione (21). 6-(3-Bromopropyl)-2,3-dimethoxy-5H-[1,3]dioxolo[4″,5″:5′,6′]pyrido[3′,2′:4,5]cyclopenta[1,2-c]isoquinoline-5,12(6H)-dione (20, 61 mg, 0.13 mmol, 1 equiv), DIPEA (0.17 g, 0.22 mL, 1.3 mmol, 10 equiv) and ethylamine (0.12 g, 1.3 mL, 2.6 mmol, 20 equiv) were dissolved in DMF (12 mL). The reaction mixture was stirred for 12 h at room temperature. More DMF (11 mL), DIPEA (0.17 g, 0.22 mL, 1.3 mmol, 10 equiv) and ethylamine (0.12 g, 1.3 mL, 2.6 mmol, 20 equiv) were added and the reaction mixture was stirred at room temperature for 12 h. The solvent was evaporated to dryness and the residue was treated with brine (100 mL) and CH₂Cl₂ (100 mL) and then stirred for 10 min. The layers were separated and the aq layer was back extracted with CH₂Cl₂ (100 mL). The combined organic extract was dried over anhydrous MgSO₄, filtered, and evaporated to dryness. The residue was purified by flash column chromatography [silica gel (24 g), eluting with 0-20% methanol in CH₂Cl₂] to furnish 6-(3-(ethylamino)propyl)-2,3-dimethoxy-5H-[1,3]dioxolo[4″,5″:5′,6′]pyrido[3′,2′:4,5]cyclopenta[1,2-c]isoquinoline-5,12(6H)-dione (21, 11 mg, 25 μmol, 20%) as a purple solid. The compound was treated with 5 equiv of 4N HCl in water (0.8 mL) and acetonitrile (0.2 mL) followed by lyophilization to afford the HCl salt 21 (11 mg, 18%). ¹H NMR (300 MHz, DMSO-d₆) δ 8.56 (s, 2H), 7.79 (s, 1H), 7.52 (s, 1H), 7.44 (s, 1H), 6.33 (s, 2H), 4.71 (t, J=6.9 Hz, 2H), 3.92 (s, 3H), 3.88 (s, 3H), 3.04-2.87 (m, 4H), 2.19-2.02 (m, 2H), 1.16 (t, J=7.2 Hz, 3H); MS (ES+): 438.4 (M+1).

6-(3-((2-Hydroxyethyl)amino)propyl)-2,3-dimethoxy-5H-[1,3]dioxolo[4″,5″:5′,6′]pyrido[3′,2′:4,5]cyclopenta[1,2-c]isoquinoline-5,12(6H)-dione (22). DIPEA (628 mg, 847 μL, 4.86 mmol, 20 equiv) was added to a stirred solution of 6-(3-bromopropyl)-2,3-dimethoxy-5H-[1,3]dioxolo[4″,5″:5′,6′]pyrido[3′,2′:4,5]cyclopenta[1,2-c]isoquinoline-5,12(6H)-dione (20, 115 mg, 243 μmol, 1 equiv) and ethanolamine (148 mg, 147 μL, 2.43 mmol, 10 equiv) in DMF (22 mL). The resulting reaction mixture was stirred for 6 h. More ethanolamine (148 mg, 147 μL, 2.43 mmol, 10 equiv) was added and the reaction mixture was stirred overnight. The solution was diluted with brine (100 mL) and extracted with CH₂Cl₂ (2×100 mL). The combined extract was dried over anhydrous MgSO₄, filtered and the solvent was evaporated to dryness. The residue was purified by flash column chromatography [silica gel (24 g), eluting with 0-100% methanol in CH₂Cl₂] to furnish 6-(3-((2-hydroxyethyl)amino)propyl)-2,3-dimethoxy-5H-[1,3]dioxolo[4″,5″:5′,6′]pyrido[3′,2′:4,5]cyclopenta[1,2-c]isoquinoline-5,12(6H)-dione (22, 11 mg, 24 μmol, 10%) as a purple solid. The product was treated with 5 equiv of 4N HCl in water (0.8 mL) and acetonitrile (0.2 mL) followed by lyophilization to afford 11 mg the HCl salt 22. ¹H NMR (300 MHz, DMSO-d₆) δ 8.58 (s, 2H), 7.82 (s, 1H), 7.54 (s, 1H), 7.46 (s, 1H), 6.33 (s, 2H), 4.72 (t, J=6.5 Hz, 2H), 3.92 (s, 3H), 3.88 (s, 3H), 3.67-3.57 (m, 2H), 3.09-2.90 (m, 4H), 2.22-2.06 (m, 2H); MS (ES+): 454.4 (M+1).

2,3-Dimethoxy-6-(3-(pyrrolidin-1-yl)propyl)-5H-[1,3]dioxolo[4″,5″:5′,6′]pyrido[3′,2′:4,5]cyclopenta[1,2-c]isoquinoline-5,12(6H)-dione (23). DIPEA (819 mg, 1.10 mL, 6.34 mmol, 10 equiv) and pyrrolidine (902 mg, 1.04 mL, 12.7 mmol, 20 equiv) were added to a stirred solution of 6-(3-bromopropyl)-2,3-dimethoxy-5H-[1,3]dioxolo[4″,5″:5′,6′]pyrido[3′,2′:4,5]cyclopenta[1,2-c]isoquinoline-5,12(6H)-dione (20, 300 mg, 634 μmol, 1 equiv) in DMF (30 mL). The reaction mixture was stirred for 16 h at room temperature. The solvent was evaporated to dryness and the residue was treated with brine (100 mL) and CH₂Cl₂ (100 mL) and then stirred for 10 min. The organic layer was separated, and the aq layer was back extracted with CH₂Cl₂ (100 mL). The combined organic extract was dried over anhydrous MgSO₄, filtered and the solvent was evaporated to dryness.

The residue was purified by flash column chromatography [silica gel (24 g), eluting with 0-20% methanol in CH₂Cl₂] to furnish 2,3-dimethoxy-6-(3-(pyrrolidin-1-yl)propyl)-5H-[1,3]dioxolo[4″,5″:5′,6′]pyrido [3′,2′:4,5]cyclopenta[1,2-c]isoquinoline-5,12(6H)-dione (23, 10 mg, 3.4%) as a purple solid. The product was mixed with water (0.8 mL) and acetonitrile (0.2 mL) followed by addition of 5 equiv of 4N aq HCl and the mixture was lyophilized to dryness to afford the HCl salt of 23 (11 mg). ¹H NMR (300 MHz, DMSO-d₆) δ 7.83 (s, 1H), 7.55 (s, 1H), 7.46 (s, 1H), 6.32 (s, 2H), 4.73 (s, 2H), 3.93 (s, 3H), 3.88 (s, 3H), 3.63-3.43 (m, 1H), 3.14-2.71 (m, 2H), 2.34-2.10 (m, 2H), 2.05-1.75 (m, 7H); MS (ES+): 464.3 (M+1).

(S)-1-(3-(2,3-dimethoxy-5,12-dioxo-5,12-dihydro-6H-[1,3]dioxolo [4″,5″:5′,6′]pyrido[3′,2′:4,5]cyclopenta[1,2-c]isoquinolin-6-yl)propyl)pyrrolidine-2-carboxamide (24). DIPEA (819 mg, 1.10 mL, 6.34 mmol, 10 equiv) and (S)-pyrrolidine-2-carboxamide (1.45 g, 12.7 mmol, 20 equiv) were added to a stirred solution of 6-(3-bromopropyl)-2,3-dimethoxy-5H-[1,3]dioxolo[4″,5″:5′,6′]pyrido[3′,2′:4,5]cyclopenta[1,2-c]isoquinoline-5,12(6H)-dione (20, 300 mg, 634 μmol, 1 equiv) in DMF (30 mL). The reaction mixture was stirred for 12 h at room temperature. The solvent was evaporated to dryness and the residue was treated with brine (100 mL) and CH₂Cl₂ (100 mL) and stirred for 10 min. The organic layer was separated, and the aq layer was back-extracted with CH₂Cl₂ (100 mL). The combined organic extract was dried over anhydrous MgSO₄, filtered and the solvent was evaporated to dryness. The residue was purified by flash column chromatography [silica gel (12 g), eluting with 0-20% methanol in CH₂Cl₂] to furnish (S)-1-(3-(2,3-dimethoxy-5,12-dioxo-5,12-dihydro-6H-[1,3]dioxolo[4″,5″:5′,6′]pyrido[3′,2′:4,5]cyclopenta[1,2-c]isoquinolin-6-yl)propyl)pyrrolidine-2-carboxamide (24, 10 mg, 20 μmol, 3.1%) as a purple solid. The solid product was mixed with water (0.8 mL) and acetonitrile (0.2 mL) then treated with 5 equiv of 4N aq HCl and lyophilized to dryness to afford the HCl salt (24). ¹H NMR (300 MHz, DMSO-d₆) δ 9.42 (s, 1H), 8.09 (s, 1H), 7.82 (s, 2H), 7.54 (s, 1H), 7.45 (s, 1H), 6.32 (s, 2H), 4.80-4.58 (m, 2H), 4.08 (dd, J=14.3, 7.1 Hz, 1H), 3.92 (s, 3H), 3.88 (s, 3H), 3.68-3.53 (m, 1H), 3.25-3.09 (m, 1H), 2.22-1.79 (m, 7H); MS (ES+): 507.3 (M+1).

(S)-6-(3-(2-(Hydroxymethyl)pyrrolidin-1-yl)propyl)-2,3-dimethoxy-5H-[1,3]dioxolo[4″,5″:5′,6′]pyrido[3′,2′:4,5]cyclopenta[1,2-c]isoquinoline-5,12(6H)-dione (25). DIPEA (819 mg, 1.10 mL, 6.34 mmol, 10 equiv) and (2S)-2-pyrrolidinemethanol (1.28 g, 1.24 mL, 12.7 mmol, 20 equiv) were added to a stirred solution of 6-(3-bromopropyl)-2,3-dimethoxy-5H-[1,3]dioxolo[4″,5″:5′,6′]pyrido[3′,2′:4,5]cyclopenta[1,2-c]isoquinoline-5,12(6H)-dione (20, 300 mg, 634 μmol, 1 equiv) in DMF (30 mL). The reaction mixture was stirred for 12 h at room temperature. The solvent was evaporated to dryness and the residue was treated with brine (100 mL) and CH₂Cl₂ (100 mL) and stirred for 10 min. The organic layer was separated and the aq layer was back-extracted with CH₂Cl₂ (100 mL). The combined organic extract was dried over anhydrous MgSO₄, filtered and the solvent was evaporated to dryness. The residue was purified by flash column chromatography [silica gel (24 g), eluting with 0-20% methanol in CH₂Cl₂] to furnish (S)-6-(3-(2-(hydroxymethyl)pyrrolidin-1-yl)propyl)-2,3-dimethoxy-5H-[1,3]dioxolo[4″,5″:5′,6′]pyrido[3′,2′:4,5]cyclopenta[1,2-c]isoquinoline-5,12(6H)-dione (25, 8 mg, 0.02 mmol, 3%) as a purple solid. The solid was mixed with water (0.8 mL) and acetonitrile (0.2 mL) and treated with 5 equiv of 4N HCl and the mixture was lyophilized to dryness to afford the HCl salt of 25 (8 mg). ¹H NMR (300 MHz, CDCl₃) δ 7.92 (s, 1H), 7.63 (s, 1H), 7.16 (d, J=2.3 Hz, 1H), 6.20 (s, 2H), 4.94-4.78 (m, 2H), 4.06 (s, 3H), 4.00 (s, 4H), 3.83-3.39 (m, 2H), 2.7-2.26 (m, 1H), 2.12-2.02 (m, 3H), 1.93-1.42 (m, 3H), 0.97-0.81 (m, 3H); MS (ES+): 494.3 (M+1).

### Method Example

### NCI-60 Cancer Cell Line Drug Screen

The antiproliferative activities of the indenoisquinoline compounds are determined in the NCI-60 cancer cell lines of the National Cancer Institute Developmental Therapeutics Program (NCI-DTP). Compounds showing sufficient cytotoxicity during the pre-screen are subjected to the five-dose assay to determine the 50% growth inhibition (GI₅₀) values. Cancer cells are incubated with the test compounds at five concentrations ranging from 100 μM to 10 nM for 48 h. After the treated cancer cells are stained with sulforhodamine B dye, the percentage growth is plotted as a function of the common logarithm of the tested compound concentration. The GI₅₀ values are determined by interpolation between the points located above and below the 50% cell growth. GI₅₀ values above and below the tested range (10-4 to 10-8 M) are taken as the maximum (10-4 M) and minimum (10-8 M) drug concentrations, respectively, used in the screening test. The approximate average of GI₅₀ values across the entire panel of NCI-60 cancer cell lines for each compound is recorded as the MGM value.

Cytotoxicity Date for G101-G105 are shown below in Tables 20a and 20b.

TABLE 20a
Antiproliferative Potencies of 7-Aza-8.9-Methylenedioxyindenoisoquinolines
in the NCI-60 Panel of Human Cancer Cell Line.

| | Cytotoxicity (GI₅₀, μM) | | | | | | | | |
|---|---|---|---|---|---|---|---|---|---|
| | Lung | Colon | CNS | Melanoma | Ovarian | Renal | Prostate | Breast | |
| compd | HOP-62 | HCT-116 | SF-539 | UACC-62 | OVCAR-3 | SN12C | DU-145 | MCF7 | MGM |
| G101 | 0.051 | 0.46 | 0.23 | 0.15 | 4.61 | 2.72 | 0.42 | 0.039 | 0.93 |
| G102 | 0.029 | 0.31 | 0.039 | 0.042 | 33.40 | 0.44 | 0.058 | 0.026 | 0.78 |
| G103 | 0.47 | 1.04 | 1.52 | 9.84 | 14.3 | 10.6 | 1.05 | 0.10 | 2.19 |
| G105 | 0.42 | 1.25 | 1.00 | 2.38 | 5.65 | 3.94 | 2.71 | 0.26 | 2.95 |
| G104 | 0.20 | 1.18 | 0.68 | 1.81 | 11.30 | 4.61 | 0.55 | 0.075 | 1.45 |
| G105 | 0.42 | 1.25 | 1 | 2.38 | 5.65 | 3.94 | 2.71 | 0.26 | 2.95 |
| G106 | 0.8 | 1.14 | 1.33 | 1.48 | 1.5 | 1.5 | 1.31 | 0.38 | 1.1 |
| G107 | 0.031 | 0.035 | 0.03 | 0.021 | 0.16 | 0.042 | 0.0388 | 0.01 | 0.0125 |
| G108 | | | | | | | | | |
| LMP744²⁰ | 0.02 | 0.1 | 0.04 | 0.03 | 0.35 | <0.01 | <0.01 | 0.79 | 0.11 |

^{a}The cyctotoxicity GI₅₀ values listed are the concentrations corresponding to 50% growth inhibition.

TABLE 20b
Comparative Cytotoxicity (GI₅₀, μM)

| | Lung | Colon | CNS | Melanoma | Ovarian | Renal | Prostate | Breast | |
|---|---|---|---|---|---|---|---|---|---|
| compd | HOP-62 | HCT-116 | SF-539 | UACC-62 | OVCAR-3 | SN12C | DU-145 | MCE7 | MGM |
| A1 | 0.35 | 1.14 | 0.76 | 2.86 | 1.42 | 1.37 | 0.76 | 0.095 | 1.32 |
| A2 | 0.22 | 0.90 | 1.05 | 1.40 | 1.38 | 1.44 | 0.79 | 0.057 | 0.87 |
| A3 | 2.38 | 1.20 | 1.51 | 13.0 | 1.64 | 1.80 | 1.44 | 0.67 | 2.69 |
| A4 | 0.80 ± | 1.14 ± | 1.33 ± | 1.48 ± | 1.50 ± | 1.64 ± | 1.31 ± | 0.38 ± | 1.10 ± |
| | 0.42 | 0.08 | 0.09 | 0.12 | 0.10 | 0.06 | 0.12 | 0.31 | 0.22 |
| A5 | 0.031 ± | 0.035 ± | 0.030 ± | 0.021 ± | 0.16 ± | 0.042 ± | 0.038 ± | 0.01 ± | 0.125 ± |
| | 0.011 | 0.014 | 0.000 | 0.008 | 0.01 | 0.001 | 0.002 | 0.00 | 0.038 |
| A6 | 1.05 ± | 0.42 ± | 1.46 ± | 1.38 ± | 1.63 ± | 1.61 ± | 1.37 ± | 0.14 ± | 1.05 ± |
| | 0.02 | 0.27 | 0.14 | 0.09 | 0.02 | 0.02 | 0.08 | 0.00 | 0.10 |
| A7 | 15.9 ± | 1.32± | 7.74 ± | 28.4 ± | 11.0 ± | 18.6 ± | 1.84 ± | 1.06 ± | 6.02 ± |
| | 1.3 | 0.26 | 6.25 | 8.75 | 1.90 | 3.9 | 0.19 | 0.14 | 0.90 |
| A8 | 0.042 ± | 0.11 ± | 0.053 ± | 0.040 ± | 0.60 ± | 0.056 ± | 0.061 ± | 0.030 ± | 0.24 ± |
| | 0.006 | 0 | 0.007 | 0.001 | 0.06 | 0.002 | 0.002 | 0.004 | 0.02 |
| A9 | 0.32 ± | 0.42 ± | 0.80 ± | 0.70 ± | 3.60 ± | 1.44 ± | 2.16± | 0.16 ± | 1.54 ± |
| | 0.30 | 0.24 | 0.58 | 0.55 | 3.07 | 1.04 | 0.90 | 0.13 | 1.09 |
| A10 | 0.67 ± | 0.40 ± | 1.00 ± | 0.84 ± | 1.82 ± | 0.44 ± | 0.96 ± | 0.16 ± | 0.86 ± |
| | 0.31 | 0.18 | 0.55 | 0.38 | 0.62 | 0.18 | 0.69 | 0.11 | 0.43 |
| A11 | 0.70 | 1.02 | 1.36 | 2.43 | 8.99 | 4.19 | 1.27 | 0.22 | 2.34 |
| A12 | 0.35 | 1.15 | 0.49 | 1.17 | 4.52 | 3.28 | 0.73 | 0.17 | 2.09 |

TABLE 20c
Structures of Compound A1-A12 of Table 20b

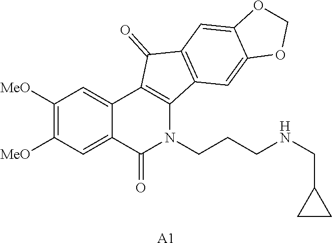 [SMILES: COc1cc2c3c(n(CCCNCC4CC4)c(=O)c2cc1OC)-c1cc2c(cc1C3=O)OCO2 ; "A1"]

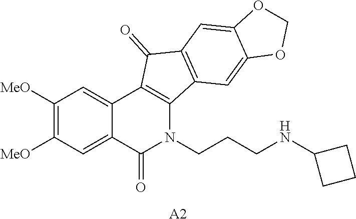

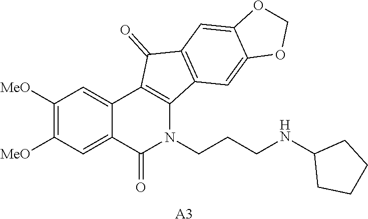 [SMILES: COc1cc2c3c(n(CCCNC4CCCC4)c(=O)c2cc1OC)-c1cc2c(cc1C3=O)OCO2 ; "A3"]

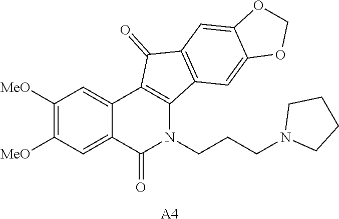

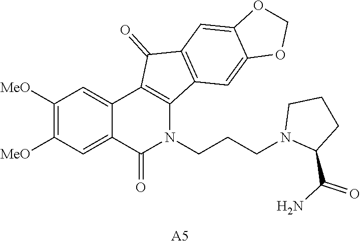 [SMILES: COc1cc2c3c(n(CCCN4CCC[C@H]4C(N)=O)c(=O)c2cc1OC)-c1cc2c(cc1C3=O)OCO2 ; "A5"]

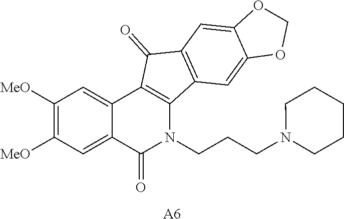

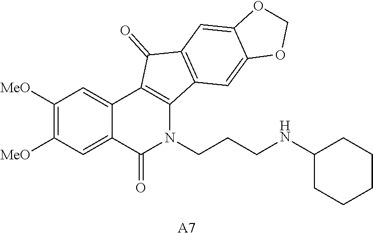 [SMILES: COc1cc2c3c(n(CCCNC4CCCCC4)c(=O)c2cc1OC)-c1cc2c(cc1C3=O)OCO2 ; "A7"]

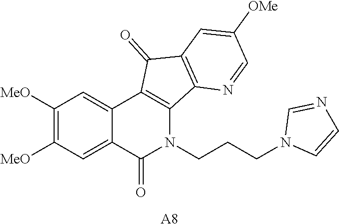

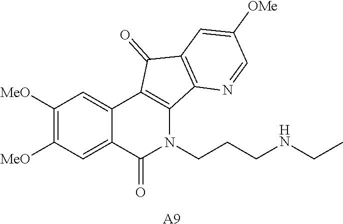

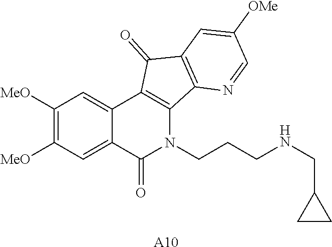 [SMILES: COc1cnc2c(c1)C(=O)c1c-2n(CCCNCC2CC2)c(=O)c2cc(OC)c(OC)cc12 ; "A10"]

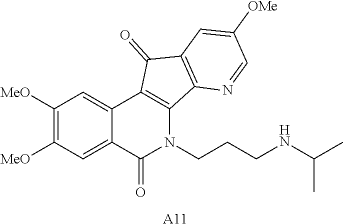

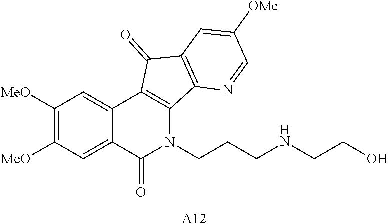 [SMILES: COc1cnc2c(c1)C(=O)c1c-2n(CCCNCCO)c(=O)c2cc(OC)c(OC)cc12 ; "A12"]

### Test Methods

DNA Melting Experiments. FRET-based DNA melting experiments were performed on a QuantStudio 6 Flex Real-Time PCR System. A 5′-fluorescein (6-FAM), 3′-rhodamine (TAMRA) dual-labeled MycG4 DNA sequence (Sigma-Aldrich) was diluted to 0.25 μM using a 7.5 mM KCl/2.5 mM potassium phosphate buffer, pH 7.0. The diluted DNA was annealed by heating to 95° C. for 2 min. Labeled MycG4 and aqueous drug solution were added to each well of a 96-well plate to a final volume of 20 μL with concentrations of 0.2 μM DNA and 2.0 μM drug in 10 mM K⁺ buffer, pH 7.0. The drug-DNA mixture was incubated in the dark at 25° C. for 1 h, and subsequently heated to 95° C. at a rate of 0.9° C./min while measuring fluorescence of 6-FAM at 520 nm (ex. 470 nm). G-quadruplex melting temperatures, Tₘ, were determined from the maximum of the first derivative of the melting curve. ΔTₘ was calculated as the difference in T_{m,DNA+drug} and T_{m,DNA}.

Fluorescence-Based Binding Assays. Fluorescence-based binding assays were performed on a Jasco FP-8300 spectrofluorometer equipped with a temperature controller at 20° C. in a quartz cuvette with a 10 mm path length. 3′-TAMRA-labeled MycG4 DNA (Sigma-Aldrich) was diluted to 20 nM using a 75 mM KCl/25 mM potassium phosphate buffer, pH 7.0. The emission spectrum of the free DNA and after each titration step was recorded from 570 to 600 nm using an excitation wavelength of 555 nm with a 5 nm bandwidth, 200 nm/min scan speed, and 1 s response time. Fluorescence intensity at 580 nm, the maximum for the free DNA, was plotted against drug:DNA ratio. The apparent binding dissociation constant, K_{d,app}, was derived for the binding of each compound. Using OriginPro software (OriginLab Corp., United States) the change in fluorescence intensity was fitted to a one-site binding equation,

$$Δ {F}_{580 n m} = {B}_{0} + \frac{( B - {B}_{0}}{2} ) \cdot ( \frac{1}{( R / {K}_{d , app} )} + L + 1 - \sqrt{{( \frac{1}{R / {K}_{d , app} )} + L + 1 )}^{2} - 4 \cdot L} )$$

where B₀ is the initial fluorescence intensity, B is maximum change in fluorescence intensity, R is DNA concentration, and L is drug:DNA ratio.

NMR Titration Experiments. NMR titration experiments were performed at 25° C. on a Bruker AV-500 spectrometer equipped with a Prodigy cryoprobe. MycG4 DNA was diluted to 150 μM in 75 mM KCl/25 mM potassium phosphate buffer, pH 7.0, in 10% D₂O/H₂O. Diluted DNA was annealed by heating to 95° C. for 2 min, and slowly cooled to room temperature. 1D ¹H NMR was measured for unbound DNA, and after subsequent titration of the compound (in DMSO-d₆). Measurements were performed at drug:DNA equivalences of 0.5, 1.0, 2.0, 3.0, 4.0. Samples were incubated for 5 min after compound addition.

Western Blotting. MCF-7 cells were grown and treated in 6-well tissue culture plates. After collection, cells were suspended in 140 μL lysis buffer containing 1× RIPA buffer, 1× Protease Inhibitor Cocktail (A32965, Thermo Scientific), 1× NuPAGE LDS Sample Buffer (NP0007, Invitrogen), and 1× Sample Reducing Agent (NP0009, Invitrogen). The cell lysates were heated to 75° C. for 10 min. After sonication, 8 μL of each sample was loaded on a 4-15% Mini-PROTEAN TGX gel (4561086, Bio-Rad). Gels were transferred to nitrocellulose membranes (IB23002, Invitrogen) using an iBlot 2 transfer device (Invitrogen). Myc and GAPDH were detected with 1:1000-diluted monoclonal anti-Myc antibody (5605S, Cell Signaling Technology) and 1:2000-diluted monoclonal anti-GAPDH antibody (5174S, Cell Signaling Technology), respectively. Primary antibodies were detected by anti-Rabbit IgG HRP-linked antibody (7074S, Cell Signaling Technology). Blotting was developed by ECL substrates (1863096, Thermo Scientific) and imaged by Bio-Rad ChemiDoc Touch Imaging System.

Quantitative Reverse Transcriptase PCR (qRT-PCR). MCF-7 cells were grown and treated in 12-well tissue culture plates until collection. Total RNA was extracted using TRIzol reagent (15596018, Life Technologies). Purified RNA was dissolved in DEPC water and quantified. 1 μg purified RNA per sample was converted to cDNA using qScript cDNA SuperMix (84034, Quantabio) according to manufacturer's instruction. Real-time PCR reagents were made for each sample by mixing 1 μL synthesized cDNA, 3 μL PCR-grade water, 5 μL SYBR Green reagent (A25742, Applied Biosystem), and 0.25 μL each primer for Myc and GAPDH. Reactions were set up in triplicate for each target of each sample. Real-time PCR was conducted in 96-well PCR plates with a QuantStudio 6 Flex Real-Time PCR System (Life Technologies). The cycling condition was 95° C. for 5 min, followed by 40 cycles at 95° C. for 15 s, 60° C. for 15 s, and 72° C. for 15 s. The relative Myc expression was calculated using the 2^{-ΔΔCt} method by normalizing against GAPDH as the endogenous control.

MTS Cell Viability IC₅₀ Assay. Drug solutions for MTS IC₅₀ assays of suspension cell lines were made from DMSO stocks by serial-dilutions in 1×PBS (10010-031, Gibco). To set up treatments, drug solutions were plated in triplicates to 96-well tissue culture plates, to which cell-containing media was added to a total volume of 100 μL/well. 100%-viability controls were untreated cells. 2000 cells/well were plated for all suspension cell lines except 3000 cells/well for KMS-27 and 10000 cells/well for CHLA-32. Blanks were cell-free media. Drug treatments were maintained for 72 hr at 37° C. with 5% CO₂. To quantify cells, CellTiter 96 AQueous MTS Reagent Powder (G1111, Promega) was dissolved and mixed with phenazine methosulfate (PMS, Sigma-Aldrich P9625) according to manufacturer's instructions. MTS/PMS mix (20 L/well) was added to treatment wells, 100%-viability control wells, and blank wells. Colors were developed at 37° C. with 5% CO₂ for 4 h and absorbance at 490 nm was measured by BioTek Synergy 4 system. To obtain %-viability, A₄₉₀ values were corrected by blank subtraction, normalized to 100%-viability control, and averaged. Average %-viabilities were plotted against drug dosages to obtain dose-response curves. Prism software (GraphPad Prism version 9.1.0 for macOS, www.graphpad.com) was used to calculate IC₅₀ using the program-implemented equation ‘log(inhibitor) vs normalized response−Variable slope’: Y=100/(1+10^{((Log IC50-X)*Hillslope)}). For adherent cell lines, the MTS IC₅₀ assays were conducted the same way as for suspension cell lines except for cell plating and drug treatment. For cell plating, 50 μL of 2000 cells were plated in triplicates to a 96-well tissue culture plate. Cells were allowed sufficient time to become adherent. Serial-diluted drug solutions were made the same way as in the MTS IC₅₀ assays. To set up treatments, drug solutions were mixed with media to two-fold of the final concentrations, and 50 μL of such drug-media mix was then added to 50 μL of plated cells.

MTS Cell Viability Single-dosage Assay. Drug solutions for the MTS single dosage assays were made from DMSO stocks by dilutions in 1×PBS. To set up treatments, drug solutions were plated in triplicates to a 96-well tissue culture plate and added with media containing CA46 or Raji 2000 cells/well. 100%-viability controls and blanks were set up the same way as in the IC₅₀ assays. Cells were quantified the same way as in the IC₅₀ assays. To obtain %-viability, A₄₉₀ values were subtracted by blank, normalized to 100%-viability control, and averaged. Prism software was used to visualize the results in bar plots.

NCI-60 Cytotoxicity Assay. Compounds were submitted to the National Cancer Institute Developmental Therapeutic Program to determine their growth-inhibitory potentials in NCI-60 cell lines. All compounds satisfied the pre-screening proceeded into the 5-dose cell viability assay, in which cells were treated for 48 h at 5 dosages ranging from 100 μM to 10 nM. After treatments, the percentage growths were plotted against the Log₁₀(drug dosages) to extrapolate the growth-inhibition at 50% (IC₅₀). GI₅₀ was reported as 100 μM or 10 nM if the GI₅₀ located above 100 μM or below 10 nM, respectively.

Pharmacokinetics. Pharmacokinetics profiles of compounds 19 (G103), 21 (G104), and 22 (G105) were determined via IV (dose 1 mg/kg) and PO (dose 10 mg/kg) administration to male CD1 mice. The mice were obtained from Si Bei Fu Laboratory Animal Technology Co. Ltd. The animals were 6-8 old and weighed 20-30 g at the time of drug administration. Plasma samples for both the IV and PO experiments were collected at 0.083, 0.25, 0.5, 1, 2, 4, 8, and 24 h. Three animals were used in each experiment, and the plasma samples were pooled together and analyzed by LC/MS/MS. The pharmacokinetics studies were conducted at Pharmaron Beijing Co. Ltd after appropriate ethical consideration and review by Pharmaron's IACUC (Institutional Animal Care and Use Committee).

Analysis of Plasma Protein Binding and Stability in Human and Mouse Blood Plasma by Equilibrium Dialysis. Frozen plasma (stored at −80° C.) was thawed immediately in a 37° C. water bath. The working solutions of test compound and control compound were prepared in DMSO at 200 μM concentration, and the working solutions were spiked into plasma. The final concentration of compound was 1 μM. The final concentration of DMSO was 0.5%. Ketoconazole was used as positive control in the assay. The dialysis membranes were soaked in ultrapure water for 60 min to separate strips, then in 20% ethanol for 20 min, and then finally in dialysis buffer for 20 min. For equilibrium dialysis, each cell contained plasma sample (150 μL) and was dialyzed against an equal volume of dialysis buffer (PBS). The assay was performed in duplicate. The dialysis plate was sealed and incubated at 37° C. with 5% CO₂ at 100 rpm for 6 h. At the end of incubation, samples (50 μL) from both buffer and plasma chambers were transferred to wells of a 96-well plate. Plasma (50 μL) was added to each buffer sample and an equal volume of PBS was added to the collected plasma sample. Cold acetonitrile (400 μL) containing internal standards (IS, 100 nM alprazolam, 200 nM labetalol, 200 nM imipramine and 2 μM ketoprofen) was added to precipitate protein and release compounds. Samples were vortexed for 2 min and centrifuged for 30 min at 3,220 g. An aliquot (100 μL) of the supernatant was diluted with ultra-pure H₂O (100 μL), and the mixture was used for LC-MS/MS analysis. For stability determination in plasma, a spiked plasma sample (50 μL) was transferred to a new plate and incubated at 37° C., 5% CO₂ for 0 and 6 h. At designated time points, PBS (50 μL) was added and mixed thoroughly, followed by the addition of room temperature quench solution [400 μL of acetonitrile containing internal standards (IS, 100 nM alprazolam, 200 nM labetalol, 200 nM imipramine and 2 μM ketoprofen)] to precipitate protein and release compounds. Samples were vortexed for 2 min and centrifuged for 30 min at 3,220 g. An aliquot (400 μL) of the supernatant was diluted by ultra-pure H₂O, and the mixture was used for LC-MS/MS analysis. The concentrations of test compounds in the buffer and plasma chambers were determined from peak area ratios.

Stability Analysis vs. Human and Mouse Liver Microsomes. Microsome solutions (0.5 mg/mL) were prepared in phosphate buffer (100 mM) containing MgCl2 (5 mM). NADPH solution (40 μL, 10 mM) was added to each well. The final concentrations of NADPH was 1 mM. The mixture was pre-warmed at 37° C. for 5 min. The negative control samples were prepared by replacing NADPH solutions with 40 μL of ultra-pure H₂O.

Samples with NADPH were prepared in duplicate and negative controls were prepared in singlet. The reaction was started by the addition control compound (2 μL of 200 μM solution) or test compound solutions. Verapamil was used as positive control. The final concentration of test compound or control compound was 1 μM. Aliquots (50 μL) were taken from the reaction solution at 0, 15, 30, 45 and 60 min. The reaction was stopped by the addition of 4 volumes of cold acetonitrile containing alprazolam (100 nM), imipramine (200 nM), labetalol (200 nM) and 2 μM ketoprofen (2 μM). Samples were centrifuged at 3,220 g for 40 min. An aliquot of the supernatant (90 μL) was mixed with ultra-pure H₂O (90 μL) and then used for LC-MS/MS analysis. Peak areas were determined from extracted ion chromatograms. The slope value, k, was determined by linear regression of the natural logarithm of the remaining percentage of the parent drug vs. incubation time curve. The in vitro half-life (in vitro t_{1/2}) was determined from the slope value: in vitro t_{1/2}=−(0.693/k). Conversion of the in vitro t_{1/2} (min) into the in vitro intrinsic clearance (in vitro CLᵢₙₜ, in μL/min/mg protein) was done using the following equation (mean of duplicate determinations):

$$in vitro {LV}_{i n t} = ( \frac{0.693}{( {t}_{1 / 2} )} ) * ( \frac{volume of incubation ( μL )}{amount of proteins ( mg )} )$$

Conversion of the in vitro t_{1/2} (min) into the scale-up unbound intrinsic clearance (Scale-up CLᵢₙₜ, in mL/min/kg) was done using the following equation (mean of duplicate determinations):

$$Scale ‐ up {CL}_{i n t} = ( \frac{0.693}{( {t}_{1 / 2} )} ) * ( \frac{volume of incubation ( mL )}{amount of proteins ( mg )} ) * Scaling Factor$$

The scaling factors used in the calculation are listed in Table 21.

TABLE 21
Scaling Factors for Intrinsic Clearance
Prediction in Liver Microsomes

| | Liver Weight | Microsomal | Liver blood | |
|---|---|---|---|---|
| | (g liver/kg | Concentration | flow (Q, | Scaling |
| Species | body weight)^{a} | (mg/g liver)^{b} | mL/min/kg)^{a} | Factor |
| Human | 25.7 | 48.8 | 20.7 | 1254.2 |
| Mouse | 87.5 | 50 | 90 | 4375 |

^{a}From reference.⁴³
^{b}From references.^{44, 45}

Caco-2 Permeability Studies. The Caco-2 plate was removed from the incubator and washed twice with pre-warmed HBSS (10 mM HEPES, pH 7.4), and then incubated at 37° C. for 30 min. The stock solutions of controls compounds were diluted in DMSO to get 1 mM solutions and then diluted with HBSS (10 mM HEPES, pH 7.4) to get 5 μM working solutions. The stock solutions of the test compounds were diluted in DMSO to get 1 mM solutions and then diluted with HBSS (10 mM HEPES, pH 7.4) to get 5 μM working solutions. The final concentration of DMSO in the incubation system was 0.5%. To determine the rate of drug transport in the apical to basolateral direction, 75 μL of 5 μM working solutions of test compounds was added to the Transwell insert (apical compartment) and the wells in the receiver plate (basolateral compartment) were filled with 235 μL of HBSS (10 mM HEPES, pH 7.4). To determine the rate of drug transport in the basolateral to apical direction, 235 μL of 5 μM working solutions of test compounds were to the receiver plate wells (basolateral compartment) and then the Transwell inserts (apical compartment) were filled with 75 μL of HBSS (10 mM HEPES, pH 7.4). Time 0 samples were prepared by transferring 50 μL of 5 μM working solution to wells of the 96-deepwell plate, followed by the addition of 200 μL cold methanol containing appropriate internal standards (IS). The plates were incubated at 37° C. for 2 h. At the end of the incubation, 50 μL samples from donor sides (apical compartment for Ap→B1 flux, and basolateral compartment for B1→Ap) and receiver sides (basolateral compartment for Ap→B1 flux, and apical compartment for B1→Ap) were transferred to wells of a new 96-well plate, followed by the addition of 4 volumes of cold acetonitrile or methanol containing appropriate internal standards (IS). Samples were vortexed for 5 min and then centrifuged at 3,220 g for 40 min. An aliquot of 100 μL of the supernatant was mixed with an appropriate volume of ultra-pure water before LC-MS/MS analysis. To determine the Lucifer Yellow leakage after 2 h transport period, stock solution of Lucifer yellow was prepared in ultra-pure water and diluted with HBSS (10 mM HEPES, pH 7.4) to reach the final concentration of 100 μM and then 100 μL of the Lucifer yellow solution was added to each Transwell insert (apical compartment), followed by filling the wells in the receiver plate (basolateral compartment) with 300 μL of HBSS (10 mM HEPES, pH 7.4). The plates were incubated at 37° C. for 30 minutes. Samples (80 μL) were removed directly from the apical and basolateral wells (using the basolateral access holes) and transferred to wells of new 96 wells plates. The Lucifer Yellow fluorescence (to monitor monolayer integrity) signal was measured in a fluorescence plate reader at 485 nM excitation and 530 nM emission.

### EMBODIMENTS

E1. A compound of formula (I), or a salt, hydrate, or solvate thereof,

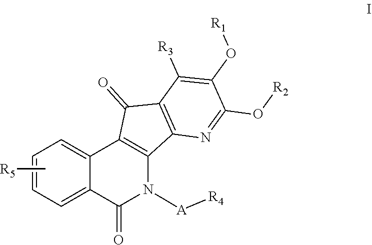

wherein
R₁ and R₂ are independently selected from the group consisting of hydrogen, (C₁-C₆) alkyl, (C₂-C₆)alkenyl, (C₂-C₆)alkynyl, optionally substituted heteroalkyl, and optionally substituted acyl, or R₁ and R₂ together with the atoms to which they are attached form a 5-membered or 6-membered ring;
R₃ is hydrogen, halo, nitro, cyan, CF₃, (C₁-C₆)alkyl, (C₁-C₆)alkylthio, or (C₁-C₆)alkoxy;
A is alkylene;
R₄ is selected from the group consisting of heteroaryl, heterocyclyl, heterocyclylamino, amino, hydroxyl, halo, cyano, alkylamino, dialkylamino, hydroxyalkylamino, bis(hydroxyalkyl)amino, and hydroxyalkylaminoalkylamino, wherein each of heteroaryl, heterocyclyl, and heterocyclylamino is optionally substituted;
R₅ represents from 1 to 2 substituents independently selected from the group consisting of amino, (C₁-C₆)alkylamino, di(C₁-C₆)alkylamino, hydroxy (C₁-C₆)alkyl, (C₁-C₆)alkenyl, (C₁-C₆)alkynyl, (C₁-C₆)heteroalkyl, (C₃-C₈)cycloalkyl, (C₃-C₈)cycloheteroalkyl, (C₁-C₆)alkoxy, (C₁-C₆)alkyl(CO)O—, (C₁-C₆)alkyl-O(CO)O— and (C₁-C₆)alkylthio; or R₅ represents 2 adjacent substituents that are taken together with the attached carbons to form an optionally substituted cycle or heterocycle.

E2. Use of the compound of E1, or a salt, hydrate, or solvate thereof, to treat a patient in need of relief from cancer.

E3. A pharmaceutical composition comprising one or more compounds of E1, or salts, hydrates, or solvates thereof, and one or more carriers, diluents, or excipients, or a combination thereof.

E4. A compound of E1, or a salt, hydrate, or solvate thereof, for treating cancer.

E5. A pharmaceutical composition comprising one or more compounds of E1, or a salt, hydrate, or solvate thereof, for treating cancer.

E6. A pharmaceutical composition for treating cancer, the composition comprising a therapeutically effective amount of one or more compounds of E1, or a salt, hydrate, or solvate thereof.

E7. A method for treating a disease responsive to topoisomerase I inhibition or binding to a MYC quadruplex in a host animal, the method comprising the step of administering to the host animal a composition comprising a therapeutically effective amount of one or more compounds of E1, or a salt, hydrate, or solvate thereof, or a pharmaceutical composition comprising one or more compounds of claim 1, or a salt, hydrate, or solvate thereof, wherein the pharmaceutical composition optionally further comprises one or more carriers, diluents, or excipients, or a combination thereof.

E8. The method of E7 wherein the host animal is a human.

E9. A method for treating cancer in a host animal, the method comprising the step of administering to the host animal a composition comprising a therapeutically effective amount of one or more compounds of claim 1, or a salt, hydrate, or solvate thereof.

E10. The method of E9 wherein the composition further comprises one or more carriers, diluents, or excipients, or a combination thereof.

E11. A process for preparing a compound of E1 comprising the step of brominating a compound of formula II to yield compound III where R₁, R₂, and R₃ are as defined in E1.

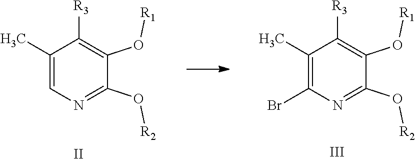

E12. The process of E11 wherein R₃ is hydrogen.

E13. The process of E11 or E12 where R₁ and R₂ are taken together to form —CH₂—.

E14. The process of any one of E11-13 wherein the brominating step comprises treating a solution of the compound of formula (II) in acetic acid with N-bromosuccinimide.

### REFERENCES

The following publications, and each of the additional publications cited herein are incorporated herein by reference:
(1) Cushman, M.; Cheng, L. Stereoselective Oxidation by Thionyl Chloride Leading to the Indeno[1,2-c]isoquinoline System. J. Org. Chem. 1978, 43, 3781-3783.
(2) Kohlhagen, G.; Paull, K.; Cushman, M.; Nagafuji, P.; Pommier, Y. Protein-Linked DNA Strand Breaks Induced by NSC 314622, a Novel Noncamptothecin Topoisomerase I Poison. Mol. Pharmacol. 1998, 54, 50-58.
(3) Ryckebusch, A.; Garcin, D.; Lansiaux, A.; Goossens, J. F.; Baldeyrou, B.; Houssin, R.; Bailly, C.; Henichart, J. P. Synthesis, Cytotoxicity, DNA Interaction, and Topoisomerase II Inhibition Properties of Novel Indeno[2,1-c]quinolin-7-one and indeno[1,2-c]isoquinolin-5,11-dione Derivatives. J. Med. Chem. 2008, 51, 3617-3629.
(4) Ahn, G.; Lansiaux, A.; Goossens, J. F.; Bailly, C.; Baldeyrou, B.; Schifano-Faux, N.; Grandclaudon, P.; Couture, A.; Ryckebusch, A. Indeno[1,2-c]Isoquinolin-5,11-diones Conjugated to Amino Acids: Synthesis, Cytotoxicity, DNA Interaction, and Topoisomerase II Inhibition Properties. Bioorg. Med. Chem. 2010, 18, 8119-8133.
(5) Ahn, G.; Schifano-Faux, N.; Goossens, J. F.; Baldeyrou, B.; Couture, A.; Grandclaudon, P.; Lansiaux, A.; Ryckebusch, A. Synthesis, Cytotoxicity and Topoisomerase Inhibition Properties of Multifarious Aminoalkylated Indeno[1,2-c]isoquinolin-5,11-diones. Bioorg. Med. Chem. Lett. 2011, 21, 2259-2263.
(6) Wambang, N.; Schifano-Faux, N.; Aillerie, A.; Baldeyrou, B.; Jacquet, C.; Bal-Mahieu, C.; Bousquet, T.; Pellegrini, S.; Ndifon, P. T.; Meignan, S.; Goossens, J. F.; Lansiaux, A.; Pelinski, L. Synthesis and Biological Activity of Ferrocenyl Indeno [1,2-c]isoquinolines as Topoisomerase II Inhibitors. Bioorg. Med. Chem. 2016, 24, 651-660.
(7) Antony, S.; Agama, K. K.; Miao, Z. H.; Hollingshead, M.; Holbeck, S. L.; Wright, M. H.; Varticovski, L.; Nagarajan, M.; Morrell, A.; Cushman, M.; Pommier, Y. Bisindenoisoquinoline Bis-1,3-{(5,6-dihydro-5,11-diketo-11H-indeno[1,2-c]isoquinoline)-6-propylamino}propane bis(trifluoroacetate) (NSC 727357), a DNA Intercalator and Topoisomerase Inhibitor with Antitumor Activity. Mol. Pharmacol. 2006, 70, 1109-1120.
(8) Park, E. J.; Kondratyuk, T. P.; Morrell, A.; Kiselev, E.; Conda-Sheridan, M.; Cushman, M.; Ahn, S.; Choi, Y.; White, J. J.; van Breemen, R. B.; Pezzuto, J. M. Induction of Retinoid X Receptor Activity and Consequent Upregulation of p21^{(wAF1/CIP1)} by Indenoisoquinolines in MCF7 Cells. Cancer Prev. Res. 2011, 4, 592-607.
(9) Chen, L.; Conda-Sheridan, M.; Reddy, P. V. N.; Morrell, A.; Park, E. J.; Kondratyuk, T. P.; Pezzuto, J. M.; van Breemen, R.; Cushman, M. Identification, Synthesis, and Biological Evaluation of the Metabolites of 3-Amino-6-(3′-aminopropyl)-5H-indeno[1,2-c]isoquinoline-5,11-(6H)dione (AM6-36), a Promising Rexinoid Lead Compound for the Development of Cancer Chemotherapeutic and Chemopreventive Agents. J. Med. Chem. 2012, 55, 5965-5981.
(10) Conda-Sheridan, M.; Park, E. J.; Beck, D. E.; Reddy, P. V. N.; Nguyen, T. X.; Hu, B.; Chen, L.; White, J. L.; Van Breemen, R. B.; Pezzuto, J. M.; Cushman, M. Design, Synthesis, and Biological Evaluation of Indenoisoquinoline Rexinoids with Chemopreventive Potential. J. Med. Chem. 2013, 56, 2581-2605.
(11) Jagtap, P. G.; Baloglu, E.; Southan, G. J.; Mabley, J. G.; Li, H. S.; Zhou, J.; van Duzer, J.; Salzman, A. L.; Szabo, C. Discovery of Potent Poly(ADP-ribose) Polymerase-1 Inhibitors from the Modification of Indeno[1,2-c] isoquinolinone. J. Med. Chem. 2005, 48, 5100-5103.
(12) Tang, Z. C.; Wu, C. Z.; Wang, T. L.; Lao, K. J.; Wang, Y. J.; Liu, L. Y.; Muyaba, M.; Xu, P.; He, C. H.; Luo, G. S.; Qian, Z. Y.; Niu, S. X.; Wang, L. J.; Wang, Y.; Xiao, H.; You, Q. D.; Xiang, H. Design, Synthesis and Evaluation of 6-Aryl-indenoisoquinolone Derivatives Dual Targeting ER Alpha and VEGFR-2 as Anti-breast Cancer Agents. Eur. J. Med. Chem. 2016, 118, 328-339.
(13) Wang, P.; Elsayed, M. S. A.; Plescia, C. B.; Ravji, A.; Redon, C. E.; Kiselev, E.; Marchand, C.; Zeleznik, O.; Agama, K.; Pommier, Y.; Cushman, M. Synthesis and Biological Evaluation of the First Triple Inhibitors of Human Topoisomerase 1, Tyrosyl-DNA Phosphodiesterase 1 (Tdp1), and Tyrosyl-DNA Phosphodiesterase 2 (Tdp2). J. Med. Chem. 2017, 60, 3275-3288.
(14) Nguyen, T. X.; Morrell, A.; Conda-Sheridan, M.; Marchand, C.; Agama, K.; Bermingam, A.; Stephen, A. G.; Chergui, A.; Naumova, A.; Fisher, R.; O'Keefe, B. R.; Pommier, Y.; Cushman, M. Synthesis and Biological Evaluation of the First Dual Tyrosyl-DNA Phosphodiesterase I (Tdp1)-Topoisomerase I (Top1) Inhibitors. J. Med. Chem. 2012, 55, 4457-4478.
(15) Conda-Sheridan, M.; Reddy, P. V. N.; Morrell, A.; Cobb, B. T.; Marchand, C.; Agama, K.; Chergui, A.; Renaud, A.; Stephen, A. G.; Bindu, L. K.; Pommier, Y.; Cushman, M. Synthesis and Biological Evaluation of Indenoisoquinolines That Inhibit Both Tyrosyl-DNA Phosphodiesterase I (Tdpl) and Topoisomerase I (TopI). J. Med. Chem. 2013, 56, 182-200.
(16) Lv, P. C.; Agama, K.; Marchand, C.; Pommier, Y.; Cushman, M. Design, Synthesis, and Biological Evaluation of O-2-Modified Indenoisoquinolines as Dual Topoisomerase I-Tyrosyl-DNA Phosphodiesterase I Inhibitors. J. Med. Chem. 2014, 57, 4324-4336.
(17) Nguyen, T. X.; Abdelmalak, M.; Marchand, C.; Agama, K.; Pommier, Y.; Cushman, M. Synthesis and Biological Evaluation of Nitrated 7-, 8-, 9-, and 10-Hydroxyindenoisoquinolines as Potential Dual Topoisomerase I (Top1)-Tyrosyl-DNA Phosphodiesterase I (TDP1) Inhibitors. J. Med. Chem. 2015, 58, 3188-3208.
(18) Wang, K. B.; Elsayed, M. S. A.; Wu, G. H.; Deng, N. J.; Cushman, M.; Yang, D. Z. Indenoisoquinoline Topoisomerase Inhibitors Strongly Bind and Stabilize the MYC Promoter G-Quadruplex and Downregulate MYC. J. Am. Chem. Soc. 2019, 141, 11059-11070.
(19) Nagarajan, M.; Morrell, A.; Antony, S.; Kohlhagen, G.; Agama, K.; Pommier, Y.; Ragazzon, P. A.; Garbett, N. C.; Chaires, J. B.; Hollingshead, M.; Cushman, M. Synthesis and Biological Evaluation of Bisindenoisoquinolines as Topoisomerase I Inhibitors. J. Med. Chem. 2006, 49, 5129-5140.
(20) Cushman, M.; Jayaraman, M.; Vroman, J. A.; Fukunaga, A. K.; Fox, B. M.; Kohlhagen, G.; Strumberg, D.; Pommier, Y. Synthesis of New Indeno[1,2-c]isoquinolines: Cytotoxic Non-Camptothecin Topoisomerase I Inhibitors. J. Med. Chem. 2000, 43, 3688-3698.
(21) Pommier, Y.; Cushman, M.; Doroshow, J. H. Novel Clinical Indenoisoquinoline Topoisomerase I Inhibitors: a Twist Around the Camptothecins. Oncotarget 2018, 9, 37286-37288.
(22) Kiselev, E.; DeGuire, S.; Morrell, A.; Agama, K.; Dexheimer, T. S.; Pommier, Y.; Cushman, M. 7-Azaindenoisoquinolines as Topoisomerase I Inhibitors and Potential Anticancer Agents. J. Med. Chem. 2011, 54, 6106-6116.
(23) Kiselev, E.; Agama, K.; Pommier, Y.; Cushman, M. Azaindenoisoquinolines as Topoisomerase I Inhibitors and Potential Anticancer Agents: A Systematic Study of Structure-Activity Relationships. J. Med. Chem. 2012, 55, 1682-1697.
(24) Kiselev, E.; Sooryakumar, D.; Agama, K.; Cushman, M.; Pommier, Y. Optimization of the Lactam Side Chain of 7-Azaindenoisoquinoline Topoisomerase I Inhibitors and Mechanism of Action Studies in Cancer Cells. J. Med. Chem. 2014, 57, 1289-1298.
(25) Elsayed, M. S. A.; Su, Y.; Wang, P.; Sethi, T.; Agama, K.; Ravji, A.; Redon, C. E.; Kiselev, E.; Horzmann, K. A.; Freeman, J. L.; Pommier, Y.; Cushman, M. Design and Synthesis of Chlorinated and Fluorinated 7-Azaindenoisoquinolines as Potent Cytotoxic Anticancer Agents That Inhibit Topoisomerase I. J. Med. Chem. 2017, 60, 5364-5376.
(26) Nagarajan, M.; Morrell, A.; Fort, B. C.; Meckley, M. R.; Antony, S.; Kohlhagen, G.; Pommier, Y.; Cushman, M. Synthesis and Anticancer Activity of Simplified Indenoisoquinoline Topoisomerase I Inhibitors Lacking Substituents on the Aromatic Rings. J. Med. Chem. 2004, 47, 5651-5661.
(27) Dallacker, F.; Fechter, P.; Mues, V. 1,3-Dioxolohetarenes. 3. Preparation and Reactions of Pyrido[2.3-d]- and Pyrido[3.4-d][1.3]dioxoles. Z. Naturforsch. B Chem. Sci. 1979, 34, 1729-1736.
(28) Vshyvenko, S.; W'Giorgis, Z.; Weber, A.; Neverova, N.; Hedberg, B.; Hudlicky, T. Synthesis and Biological Activity of 10-Aza-narciclasine. Adv. Syn. Catal. 2015, 357, 83-87.
(29) Falb, E.; Ulanenko, K.; Tor, A.; Gottesfeld, R.; Weitman, M.; Afri, M.; Gottlieb, H.; Hassner, A. A Highly Efficient Suzuki-Miyaura Methylation of Pyridines Leading to the Drug Pirfenidone and Its CD₃ Version (SD-560). Green Chem. 2017, 19, 5046-5053.
(30) Li, H. J.; Wu, Y. C.; Dai, J. H.; Song, Y.; Cheng, R. J.; Qiao, Y. Y. Regioselective Electrophilic Aromatic Bromination: Theoretical Analysis and Experimental Verification. Molecules 2014, 19, 3401-3416.
(31) Fukuda, T.; Iwao, M. Synthesis of Both Enantiomers of Protoberberines via Laterally Lithiated (S)-4-Isopropyl-2-(o-tolyl)oxazolines. Heterocycles 2007, 74, 701-720.
(32) Wang, B. J.; Qin, L. L.; Neumann, K. D.; Uppaluri, S.; Cerny, R. L.; DiMagno, S. G. Improved Arene Fluorination Methodology for I(III) Salts. Organic Lett. 2010, 12, 3352-3355.
(33) Jagtap, P. G.; Baloglu, E.; Southan, G.; Williams, W.; Roy, A.; Nivorozhkin, A.; Landrau, N.; Desisto, K.; Salzman, A. L.; Szab6, C. Facile and Convenient Syntheses of 6,11-Dihydro-5H-indeno [1,2-c]isoquinolin-5-ones and 6,11-Dihydro-5H-indolo[3,2-c]isoquinolin-5-one. Org. Lett. 2005, 7, 1753-1756.
(34) Beck, D. E.; Lv, W.; Abdelmalak, M.; Plescia, C. B.; Agama, K.; Marchand, C.; Pommier, Y.; Cushman, M. Synthesis and Biological Evaluation of New Fluorinated and Chlorinated Indenoisoquinoline Topoisomerase I Poisons. Bioorg. Med. Chem. 2016, 24, 1469-1479.
(35) Marzi, L.; Agama, K.; Murari, J.; Difilippantonio, S.; James, A.; Peer, C. J.; Figg, W. D.; Beck, D. E.; Elsayed M. S. A.; Cushman, M.; et al. Novel Fluoroindenoisoquinoline Non-Camptothecin Topoisomerase I Inhibitors. Mol. Cancer Ther. 2018, 17, 1694-1704.
(36) Marzi, L.; Sun, Y. L.; Huang, S. Y. N.; James, A.; Difilippantonio, S.; Pommier, Y. The Indenoisoquinoline LMP517: A Novel Antitumor Agent Targeting both TOP1 and TOP2. Mol. Cancer Ther. 2020, 19, 1589-1597.
(37) Shoemaker, R. H. The NCI60 Human Tumour Cell Line Anticancer Drug Screen. Nat. Rev. Cancer 2006, 6, 813-823.
(38) Strumberg, D.; Pommier, Y.; Paull, K.; Jayaraman, M.; Nagafuji, P.; Cushman, M. Synthesis of Cytotoxic Indenoisoquinoline Topoisomerase I Poisons. J. Med. Chem. 1999, 42, 446-457.
(39) Stacker, B. L.; Feese, M. D.; Cushman, M.; Pommier, Y.; Zembower, D.; Stewart, L.; Burgin, A. B. Structures of Three Classes of Anticancer Agents Bound to the Human Topoisomerase I-DNA Covalent Complex. J. Med. Chem. 2005, 48, 2336-2345.
(40) Gao, J.; Holleran, J.; Schmitz, J. C.; Czambel, K.; Beumer, J. H.; Eiseman, J. L. Abstract 4513: Pharmacokineetic and Pharmacodynamics of Indenoisoquinoline LMP400 (Indotecan) in BALB/s Female Mice Bearing CT-26 Colon Tumors. In Proceedings of the 106^{th} Annual Meeting of the American Association for Cancer Research, 2015.
(41) Weight of Brain. https://www.aqua-calc.com/calculate/volume-to-weight/substance/brain (accessed Mar. 22, 2023).
(42) Eisman, J. L.; Holeran, J.; McCormick, D. L.; Muzzio, M.; Covey, J. M.; Khanna, C.; Mazcko, C.; Pommier, Y.; Paolini, M.; Leblanc, A.; et al. Abstract 4517: Plasma and Tumor Pharmacokinetics of IV LMP744, a Novel Indenoisoquinoline Topoisomerase I Inhibitor, in a Canine Phase I Study. Cancer Res. 2015, 75 (15_Supplement), 4517-5417.
(43) Davies, B.; Morris, T. Physiological Parameters in Laboratory Animals and Humans. Pharm. Res. 1993, 10, 1093-1095.
(44) Iwatsubo, T.; Suzuki, H.; Sugiyama, Y. Prediction of Species Differences (Rats, Dogs, Humans) in the In Vivo Metabolic Clearance of YM796 by the Liver from In Vitro Data. J. Pharm. Exp. Ther. 1997, 283, 462-469.
(45) Barter, Z. E.; Bayliss, M. K.; Beaune, P. H.; Boobis, A. R.; Carlile, D. J.; Edwards, R. J.; Houston, J. B.; Lake, B. G.; Lipscomb, J. C.; Pelkonen, O. R.; Tucker, G. T.; Rostami-Hodjegan, A. Scaling Factors for the Extrapolation of In Vivo Metabolic Drug Clearance from In Vitro Data: Reaching a Consensus on Values of Human Microsomal Protein and Hepatocellularity Per Gram of Liver. Curr. Drug Metab. 2007, 8, 33-45.

What is claimed is:

1. A compound of formula (I), or a salt, hydrate, or solvate thereof, 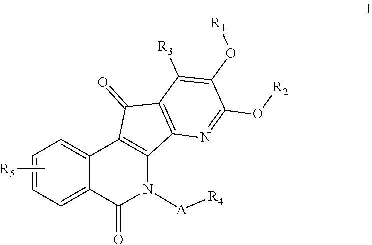
wherein
R₁ and R₂ are independently selected from the group consisting of hydrogen, (C₁-C₆) alkyl, (C₂-C₆)alkenyl, (C₂-C₆)alkynyl, optionally substituted heteroalkyl, and optionally substituted acyl, or R₁ and R₂ together with the atoms to which they are attached form a 5-membered or 6-membered ring;
R₃ is hydrogen, halo, nitro, cyan, CF₃, (C₁-C₆)alkyl, (C₁-C₆)alkylthio, or (C₁-C₆)alkoxy;
A is alkylene;
R₄ is selected from the group consisting of heteroaryl, heterocyclyl, heterocyclylamino, amino, hydroxyl, halo, cyano, alkylamino, dialkylamino, hydroxyalkylamino, bis(hydroxyalkyl)amino, and hydroxyalkylaminoalkylamino, wherein each of heteroaryl, heterocyclyl, and heterocyclylamino is optionally substituted;
R₅ represents from 1 to 2 substituents independently selected from the group consisting of amino, (C₁-C₆)alkylamino, di(C₁-C₆)alkylamino, hydroxy (C₁-C₆)alkyl, (C₁-C₆)alkenyl, (C₁-C₆)alkynyl, (C₁-C₆)heteroalkyl, (C₃-C₈)cycloalkyl, (C₃-C₈)cycloheteroalkyl, (C₁-C₆)alkoxy, (C₁-C₆)alkyl(CO)O—, (C₁-C₆)alkyl-O(CO)O— and (C₁-C₆)alkylthio; or R₅ represents 2 adjacent substituents that are taken together with the attached carbons to form an optionally substituted cycle or heterocycle.

2. The compound of claim 1, wherein A is (CH₂)n, wherein n is selected from the group consisting of 1, 2 and 3.

3. The compound of claim 2, wherein A is (CH₂)₃.

4. The compound of claim 1, wherein R₃ is hydrogen.

5. The compound of claim 1, wherein R₁ and R₂ are independently selected from the group consisting of hydrogen and CH₃.

6. The compound of claim 1, wherein R₁ and R₂ taken together with atoms they are attached to form a 5- or 6-membered ring.

7. The compound of claim 1, wherein R₄ is selected from the group consisting of heteroaryl, heterocyclyl, heterocyclylamino, amino, hydroxyl, halo, cyano, alkylamino, dialkylamino, hydroxyalkylamino, bis(hydroxyalkyl)amino, and hydroxyalkylaminoalkylamino, wherein each of heteroaryl, heterocyclyl, and heterocyclylamino is optionally substituted.

8. The compound of claim 1, wherein R₁ and R₂ are taken together to form —CH₂—, R₃ is hydrogen, R₅ represents 2-MeO and 3-MeO, A is (CH₂)₃, and R₄ is selected from the group consisting of heteroaryl, heteroaryloxy, heteroarylamino, heteroarylalkylaminoalkylamino, heterocyclyl, heterocyclylamino, amino, hydroxyl, halo, cyano, alkylamino, dialkylamino, trialkylammonium, hydroxyalkylamino, bis(hydroxyalkyl)amino, and hydroxyalkylaminoalkylamino, wherein each of heteroaryl, heteroaryloxy, and heteroarylamino, heteroarylalkylaminoalkylamino, heterocyclyl, and heterocyclylamino is optionally substituted.

9. The compound of claim 1, wherein the compound is selected from the group consisting of

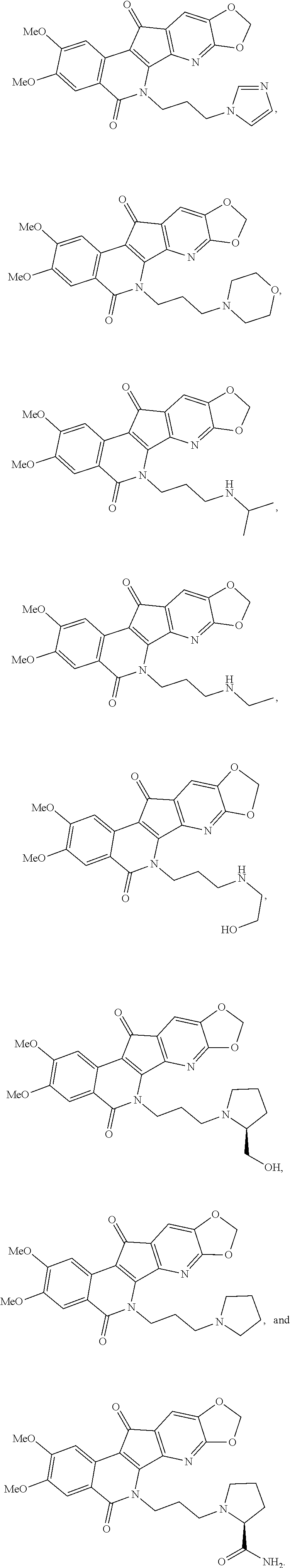

10. A pharmaceutical composition comprising the compound of claim 1, or the salt, hydrate, or solvate thereof.

11. A pharmaceutical composition comprising a compound of formula (I), or a salt, hydrate, or solvate thereof 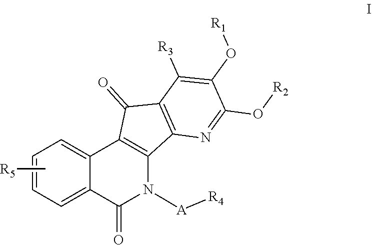
wherein
R₁ and R₂ are independently selected from the group consisting of hydrogen, (C₁-C₆) alkyl, (C₂-C₆)alkenyl, (C₂-C₆)alkynyl, optionally substituted heteroalkyl, and optionally substituted acyl, or R₁ and R₂ together with the atoms to which they are attached form a 5-membered or 6-membered ring;
R₃ is hydrogen, halo, nitro, cyan, CF₃, (C₁-C₆)alkyl, (C₁-C₆)alkylthio, or (C₁-C₆)alkoxy;
A is (CH₃)ₙ wherein n is from 2 to 3;
R₄ is selected from the group consisting of heteroaryl, heterocyclyl, heterocyclylamino, amino, hydroxyl, halo, cyano, alkylamino, dialkylamino, hydroxyalkylamino, bis(hydroxyalkyl)amino, and hydroxyalkylaminoalkylamino, wherein each of heteroaryl, heterocyclyl, and heterocyclylamino is optionally substituted;
R₅ represents from 1 to 2 substituents independently selected from the group consisting of amino, (C₁-C₆)alkylamino, di(C₁-C₆)alkylamino, hydroxy (C₁-C₆)alkyl, (C₁-C₆)alkenyl, (C₁-C₆)alkynyl, (C₁-C₆)heteroalkyl, (C₃-C₈)cycloalkyl, (C₃-C₈)cycloheteroalkyl, (C₁-C₆)alkoxy, (C₁-C₆)alkyl(CO)O—, (C₁-C₆)alkyl-O(CO)O— and (C₁-C₆)alkylthio; or R₅ represents 2 adjacent substituents that are taken together with the attached carbons to form an optionally substituted cycle or heterocycle.

12. The pharmaceutical composition of claim 11, further comprising at least one additional component selected from the group consisting of a diluent, an excipient, and combinations thereof.

13. The pharmaceutical composition of claim 11 for treating cancer.

14. The pharmaceutical composition of claim 11, the composition comprising a therapeutically effective amount of the compound of formula (I), or salt, hydrate, or solvate thereof.

15. A method for treating a disease responsive to topoisomerase I inhibition or binding to a MYC quadruplex in a host animal, the method comprising the step of administering to the host animal a composition comprising a therapeutically effective amount of one or more compounds of formula (I), or a salt, hydrate, or solvate thereof, or a pharmaceutical composition comprising one or more compounds of formula (I), or a salt, hydrate, or solvate thereof,
wherein formula (I) is 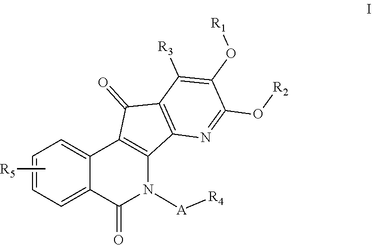
wherein
R₁ and R₂ are independently selected from the group consisting of hydrogen, (C₁-C₆) alkyl, (C₂-C₆)alkenyl, (C₂-C₆)alkynyl, optionally substituted heteroalkyl, and optionally substituted acyl, or R₁ and R₂ together with the atoms to which they are attached form a 5-membered or 6-membered ring;
R₃ is hydrogen, halo, nitro, cyan, CF₃, (C₁-C₆)alkyl, (C₁-C₆)alkylthio, or (C₁-C₆)alkoxy;
A is alkylene;
R₄ is selected from the group consisting of heteroaryl, heterocyclyl, heterocyclylamino, amino, hydroxyl, halo, cyano, alkylamino, dialkylamino, hydroxyalkylamino, bis(hydroxyalkyl)amino, and hydroxyalkylaminoalkylamino, wherein each of heteroaryl, heterocyclyl, and heterocyclylamino is optionally substituted;
R₅ represents from 1 to 2 substituents independently selected from the group consisting of amino, (C₁-C₆)alkylamino, di(C₁-C₆)alkylamino, hydroxy (C₁-C₆)alkyl, (C₁-C₆)alkenyl, (C₁-C₆)alkynyl, (C₁-C₆)heteroalkyl, (C₃-C₈)cycloalkyl, (C₃-C₈)cycloheteroalkyl, (C₁-C₆)alkoxy, (C₁-C₆)alkyl(CO)O—, (C₁-C₆)alkyl-O(CO)O— and (C₁-C₆)alkylthio; or R₅ represents 2 adjacent substituents that are taken together with the attached carbons to form an optionally substituted cycle or heterocycle, and
wherein the pharmaceutical composition optionally further comprises one or more carriers, diluents, or excipients, or a combination thereof.

16. The method of claim 15, wherein the host animal is a human.

17. A process for preparing a compound of claim 1 comprising the step of brominating a compound of formula II to yield compound III where R₁, R₂, and R₃ are as defined in claim 1.

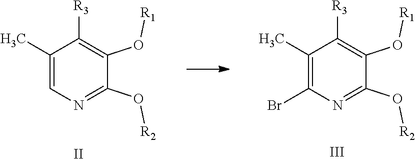

18. The process of claim 17 wherein R₃ is hydrogen.

19. The process of claim 18, where R₁ and R₂ are taken together to form —CH₂—.

20. The process of claim 19, wherein the brominating step comprises treating a solution of the compound of formula (II) in acetic acid with N-bromosuccinimide.
